(19) United States
(12) Patent Application Publication
(10) Pub. No.: US 2012/0015957 A1
(43) Pub. Date: Jan. 19, 2012

(54) PIPERAZINYL DERIVATIVES USEFUL AS MODULATORS OF THE NEUROPEPTIDE Y2 RECEPTOR

(76) Inventors: Wenying Chai, San Diego, CA (US); Curt A. Dvorak, Poway, CA (US); Jill A. Jablonowski, San Diego, CA (US); Dale A. Rudolph, San Diego, CA (US); Chandravadan R. Shah, San Diego, CA (US); Victoria D. Wong, San Diego, CA (US)

(21) Appl. No.: 12/809,010

(22) PCT Filed: Dec. 17, 2008
(86) PCT No.: PCT/US08/87250

§ 371 Date: Jul. 26, 2010

Related U.S. Application Data
(60) Provisional application No. 61/014,176, filed on Dec. 17, 2007.

(51) Int. Cl. A61K 31/496; C07D 403/10; C07D 413/14; C07D 295/15; A61K 31/495; C07D 401/10; C07D 417/10; A61P 25/22; A61P 25/24; A61P 25/00; A61P 19/08; A61P 9/00; A61P 25/30; A61P 3/04; A61P 5/00; A61P 15/08; A61P 25/32; C07D 413/10

(52) U.S. Cl. 514/253.1; 544/367; 514/254.03; 544/369; 514/254.02; 514/254.04; 544/371; 514/254.05; 544/372; 514/254.01; 544/364; 544/393; 514/255.03; 544/360; 514/253.01; 544/366

(57) ABSTRACT

The present invention is directed to piperidinyl and piperazinyl derivatives of formula (I) useful as inhibitors of the NPY Y2 receptor, pharmaceutical compositions comprising said compounds, processes for the preparation of said compounds and the use of said compounds for the treatment and/or prevention of disorders, diseases and conditions mediated by the NPY Y2 receptor.

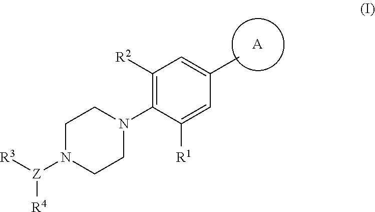

### FIELD OF THE INVENTION

The present invention is directed to piperazinyl derivatives useful as inhibitors of the NPY Y₂ receptor, pharmaceutical compositions comprising said compounds, processes for the preparation of said compounds and the use of said compounds for the treatment and/or prevention of disorders, diseases and conditions mediated by the NPY Y₂ receptor, including, but not limited to anxiolytic disorders, depression; pain, injured mammalian nerve tissue; conditions responsive to treatment with a neurotrophic factor; neurological disorders; bone loss; cardiovascular diseases; sleep-wake state disorders, substance abuse and addiction related disorders; obesity; and obesity-related disorders. The compounds of the present invention are further useful in modulating endocrine functions; particularly endocrine functions controlled by the pituitary and hypothalamic glands, and are therefore useful in the treatment of metabolic disorders, inovulation and infertility.

### BACKGROUND OF THE INVENTION

Regulation and function of the mammalian central nervous system is governed by a series of interdependent receptors, neurons, neurotransmitters, and proteins. The neurons play a vital role in this system, for when externally or internally stimulated, they react by releasing neurotransmitters that bind to specific proteins. Common examples of endogenous small molecule neurotransmitters such as acetylcholine, adrenaline, norepinephrine, dopamine, serotonin, glutamate, and gamma-aminobutyric acid are well known, as are the specific receptors that recognize these compounds as ligands (“The Biochemical Basis of Neuropharmacology”, Sixth Edition, Cooper, J. R.; Bloom, F. E.; Roth, R. H. Eds., Oxford University Press, New York, N.Y. 1991).

In addition to the endogenous small molecule neurotransmitters, there is increasing evidence that neuropeptides play an integral role in neuronal operations. Neuropeptides are now believed to be co-localized with perhaps more than one-half of the 100 billion neurons of the human central nervous system. In addition to being found in humans, neuropeptides have been discovered in a number of animal species. In some instances, the composition of these peptides is remarkably homogenous among species. This finding suggests that the function of neuropeptides is vital and has been impervious to evolutionary changes. Furthermore, neuropeptides, unlike small molecule neurotransmitters, are typically synthesized by the neuronal ribosome. In some cases, the active neuropeptides are produced as part of a larger protein that is enzymatically processed to yield the active substance. Based upon these differences, compared to small molecule neurotransmitters, neuropeptide-based strategies may offer novel therapies for the treatment of CNS diseases and disorders. Specifically, agents that affect the binding of neuropeptides to their respective receptors or that ameliorate responses that are mediated by neuropeptides are potential therapies for diseases associated with neuropeptides.

There are a number of afflictions that are associated with the complex interdependent system of receptors and ligands within the central nervous system; these include neurodegenerative diseases, affective disorders such as anxiety, depression, pain and schizophrenia, and affective conditions that include a metabolic component, namely obesity. Such conditions, disorders, and diseases have been treated with small molecules and peptides that modulate neuronal responses to endogenous neurotransmitters.

One example of this class of neuropeptides is neuropeptide Y (NPY). NPY was first isolated from porcine brain (Tatemoto, K. et al. Nature 1982, 296, 659) and was shown to be structurally similar to other members of the pancreatic polypeptide (PP) family such as peptide YY (PYY), which is primarily synthesized by endocrine cells in the gut, and pancreatic polypeptide, which is synthesized by the pancreas. NPY is a single peptide protein that consists of thirty-six amino acids containing an amidated C-terminus. Like other members of the pancreatic polypeptide family, NPY has a distinctive conformation that consists of an N-terminal polyproline helical region and an amphiphilic alpha-helix joined by a characteristic PP-fold (Vladimir, S. et al. Biochemistry 1990, 20, 4509). Furthermore, NPY sequences from a number of animal species have been elucidated and all show a high degree of amino acid homology to the human protein (more than 94% in rat, dog, rabbit, pig, cow, sheep) (see Larhammar, D. in “The Biology of Neuropeptide Y and Related Peptides”, Colmers, W. F. and Wahlestedt, C. Eds., Humana Press, Totowa, N.J. 1993).

Endogenous receptor proteins that bind NPY and related peptides as ligands have been identified and distinguished, and several such proteins have been cloned and expressed. Six different receptor subtypes [Y1, Y2, Y3, Y4(PP), Y5, Y6 (formerly designated as a Y5 receptor)] are recognized based upon binding profile, pharmacology, and/or composition if identity is known (Wahlestedt, C. et al. Ann. N.Y. Acad. Sci. 1990, 611, 7; Larhammar, D. et al. J. Biol. Chem. 1992, 267, 10935; Wahlestedt, C. et al. Regul. Pept. 1986, 13, 307; Fuhlendorff, J. U. et al. Proc. Natl. Acad. Sci. U.S.A. 1990, 87, 182; Grundemar, L. et al. J. Pharmacol. Exp. Ther. 1991, 258, 633; Laburthe, M. et al. Endocrinology 1986, 118, 1910; Castan, I. et al. Endocrinology 1992, 131, 1970; Gerald, C. et al. Nature 1996, 382, 168; Weinberg, D. H. et al. J. Biol. Chem. 1996, 271, 16435; Gehlert, D. et al. Curr. Pharm. Des. 1995, 1, 295; Lundberg, J. M. et al. Trends Pharmacol. Sci. 1996, 17, 301). Most and perhaps all NPY receptor proteins belong to the family of so-called G-protein coupled receptors (GPCRs).

NPY itself is the archetypal substrate for the NPY receptors and its binding can elicit a variety of pharmacological and biological effects in vitro and in vivo. When administered to the brain of live animals (intracerebro-ventricularly (icy) or into the amygdala), NPY produced anxiolytic effects in established animal models of anxiety such as the elevated plus-maze, Vogel punished drinking, and Geller-Seifter's bar-pressing conflict paradigms (Heilig, M. et al. Psychopharmacology 1989, 98, 524; Heilig, M. et al. Regul. Pept. 1992, 41, 61; Heilig, M. et al. Neuropsychopharmacology 1993, 8, 357). Thus, compounds that mimic NPY are postulated to be useful for the treatment of anxiolytic disorders. The immunoreactivity of NPY is notably decreased in the cerebrospinal fluid of patients with major depression and those of suicide victims (Widdowson, P. S. et al. J. Neurochem. 1992, 59, 73), and rats treated with tricyclic antidepressants displayed significant increases of NPY relative to a control group (Heilig, M. et al. Eur. J. Pharmacol. 1988, 147, 465). These findings suggest that an inadequate NPY response may play a role in some depressive illnesses, and that compounds that regulate the NPY-ergic system may be useful for the treatment of depression.

It is known that the anxiolytic properties of NPY are mediated through postsynaptic Y1 receptors, whereas presynaptic Y2 receptors negatively control the release of NPY and other cotransmitters (e.g. GABA). Consequently, antagonism of the Y2 receptor may lead to enhanced GABAergic and NPYergic effects and Y2 receptor antagonists should prove useful in the treatment of depression and anxiety.

NPY improved memory and performance scores in animal models of learning (Flood, J. F. et al. Brain Res. 1987, 421, 280) and therefore may serve as a cognition enhancer for the treatment of neurodegenerative diseases such as Alzheimer's Disease (AD) as well as AIDS-related and senile dementia.

Elevated plasma levels of NPY were present in animals and humans experiencing episodes of high sympathetic nerve activity such as surgery, newborn delivery, and hemorrhage (Morris, M. J. et. al. J. Auton. Nerv. Syst. 1986, 17, 143). Thus, chemical substances that alter the NPY-ergic system may be useful for alleviating migraine, pain, and the condition of stress.

NPY also mediates endocrine functions such as the release of luteinizing hormone (LH) in rodents (Kalra, S. P. et. al. Front. Neuroendrocrinol. 1992, 13, 1). Since LH is vital for mammalian ovulation, a compound that mimics the action of NPY could be useful for the treatment of infertility, particularly in women with so-called luteal phase defects.

NPY is a powerful stimulant of food intake; as little as one-billionth of a gram, when injected directly into the CNS, caused satiated rats to overeat (Clark, J. T. et al. Endocrinology 1984, 115, 427; Levine, A. S. et al. Peptides 1984, 5, 1025; Stanley, B. G. et al. Life Sci. 1984, 35, 2635; Stanley, B. G. et al. Proc. Nat. Acad. Sci. U.S.A. 1985, 82, 3940). Thus NPY is orexigenic in rodents but not anxiogenic when given intracerebroventricularly and so antagonism of neuropeptide receptors may be useful for the treatment of diabetes and eating disorders such as obesity, anorexia nervosa, and bulimia nervosa.

Recently, a key role of presynaptic hypothalamic Y2 receptor was suggested in central coordination of energy homeostasis and bone mass regulation (Herzog, H. et al. Drug News & Perspectives 2002, 15, 506-510). Studies analyzing Y2 receptor knockout mice have started to unravel some of the individual functions of this receptor subtype. Y2 receptor knockout mice showed a reduced body weight despite an increase in food intake, possibly due to the lack of the feedback inhibition of the postprandially released PYY₃₋₃₆ (Batterham, R. L. et al. Nature 2002, 418, 650-654). The Y2 receptor knockout mice also showed a significant increase in bone formation (Baldock, P. A. J. Clin. Invest. 2002, 109, 915-921). Specific deletion of the Y2 receptor in the hypothalamus in adult conditional Y2 receptor knockout mice also caused an increase in bone formation.

Studies have also indicates that NPY Y2 is involved in the neurobiological responses to ethanol and other drugs of abuse. Thiele and coworkers (Neuropeptides, 2004, 38(4), 235-243; Peptides 2004, 25(6), 975-983) described the low ethanol consumption of Y2 receptor knockout mice, as well as their increased voluntary water consumption. Therefore, modulators of NPY Y2 may allow for the treatment of alcohol and drug abuse.

Grouzmann and coworkers described a peptide-based ligand, T4-[NPY 33-36], which showed considerable affinity (IC₅₀=67 nM) for the NPY Y2 receptor (Grouzmann, E., et al. J. Biol. Chem. 1997, 272, 7699-7706). BIIE0246 also bound to the NYP Y2 receptor with significant affinity (IC₅₀=3.3 nM) (Doods, H., et al. Eur. J. Pharmacol. 1999, 384, R3-R5). However, the therapeutic potential for these compounds is limited due to their peptide-like composition and elevated molecular weight.

There remains however, a need for potent NPY Y2 modulators with desirable pharmaceutical properties.

### SUMMARY OF THE INVENTION

The present invention is directed to piperazinyl derivatives, compounds of formula (I):

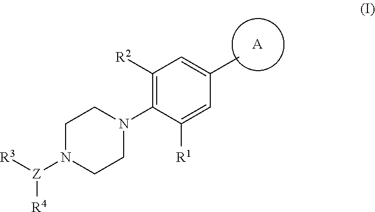

wherein

R¹ and R² are each independently selected from the group consisting of hydrogen, halogen, C₁₋₄alkyl, —C₁₋₄alkyl-OH, —C₁₋₄alkyl-O—C₁₋₄alkyl, —C₁₋₄alkoxy, —S—C₁₋₄alkyl, —SO—C₁₋₄alkyl, —SO₂—C₁₋₄alkyl, cyano, nitro, —NR^{A}R^{B}, —CH₂—NR^{A}R^{B}, —C(O)—NR^{A}R^{B} and —C(O)H; wherein R^{A} and R^{B} are each independently selected from the group consisting of hydrogen and C₁₋₄alkyl; provided that at least one or R¹ or R² is other than hydrogen;

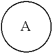

is selected from the group consisting of cycloalkyl, aryl, heteroaryl and heterocycloalkyl; wherein the cycloalkyl, aryl, heteroaryl or heterocycloalkyl is optionally substituted with one or more substituents independently selected from the group consisting of halogen, C₁₋₈alkyl, halogenated C₁₋₄alkyl, C₁₋₄alkoxy, —S—C₁₋₄alkyl, —SO—C₁₋₄alkyl, —SO₂—C₁₋₄alkyl, cyano, oxo, C₃₋₈cycloalkyl, phenyl, —C(O)OH, —C(O)O—C₁₋₄alkyl, —C(O)—NR^{C}R^{D} and —C(NR^{C}R^{D})═N—OH; wherein R^{C} and R^{D} are each independently selected from the group consisting of hydrogen and C₁₋₄alkyl;

provided that

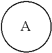

is other than 1-(pyrrolidin-2-one), 2-([1,2,5]-thiadiazolidine-1,1-dioxide), 2-(5-n-propyl-[1,2,5]-thiadiazolidine-1,1-dioxide), 2-(5-isopropyl-[1,2,5]-thiadiazolidine-1,1-dioxide), 2-(5-cyclopentyl-[1,2,5]-thiadiazolidine-1,1-dioxide) and 2-(5-(methoxycarbonyl)-[1,2,5]-thiadiazolidine-1,1-dioxide);

Z is selected from the group consisting of CH and CR⁰; wherein R⁰ is selected from the group consisting of —C₁₋₄alkyl;

R³ is selected from the group consisting of C₁₋₄alkyl, C₂₋₄alkenyl, cyano, C₃₋₈cycloalkyl, aryl, C₁₋₄aralkyl and 5 to 6 membered heteroaryl; wherein the aryl or heteroaryl, whether alone or as part of a substituent group is optionally substituted with one to two substituents independently selected from the group consisting of halogen, hydroxy, C₁₋₄alkyl, halogenated C₁₋₄alkyl, C₁₋₄alkoxy, halogenated C₁₋₄alkoxy, cyano, nitro, —NR^{E}R^{F} and —C(O)—NR^{E}R^{F}; wherein R^{E} and R^{F} are each independently selected from the group consisting of hydrogen and C₁₋₄alkyl;

R⁴ is selected from the group consisting of cyano, C₁₋₈alkyl, —C₁₋₄alkyl-OH, C₁₋₄alkyl-CN, —C(O)—NR^{G}R^{H}, —C(O)—C₁₋₄alkyl, —C(O)—O—C₁₋₄alkyl; wherein R^{G} and R^{H} are each independently selected from the group consisting of hydrogen and C₁₋₄alkyl; alternatively, R^{G} and R^{H} are taken together with the nitrogen atom to which they are bound to form a 4 to 8 membered saturated ring structure; wherein the 4 to 8 membered saturated ring structure is optionally substituted with one or more halogen;

and enantiomers and pharmaceutically acceptable salts thereof.

Illustrative of the invention is a pharmaceutical composition comprising a pharmaceutically acceptable carrier and any of the compounds described herein. An illustration of the invention is a pharmaceutical composition made by mixing any of the compounds described herein and a pharmaceutically acceptable carrier. Illustrating the invention is a process for making a pharmaceutical composition comprising mixing any of the compounds described herein and a pharmaceutically acceptable carrier.

Exemplifying the invention are methods of treating a disorder mediated by the neuropeptide Y₂ receptor (selected from the group consisting of anxiolytic disorders, depression; pain, injured mammalian nerve tissue; conditions responsive to treatment with a neurotrophic factor; neurological disorders; bone loss; cardiovascular diseases; sleep-wake state disorders, substance abuse and addiction related disorders; obesity; obesity-related disorders, disorders responsive to modulation of endocrine function, inovulation and infertility; comprising administering to a subject in need thereof a therapeutically effective amount of any of the compounds or pharmaceutical compositions described above.

Another example of the invention is the use of any of the compounds described herein in the preparation of a medicament for treating: (a) anxiolytic disorders, (b) depression; (c) pain, (d) injured mammalian nerve tissue; (d) conditions responsive to treatment with a neurotrophic factor; (e) neurological disorders; (f) bone loss; (g) cardiovascular diseases; (h) sleep-wake state disorders, (i) substance abuse and addiction related disorders; (j) obesity; (k) obesity-related disorders, (l) disorders responsive to modulation of endocrine function (more particularly, disorders responsive to modulation of the pituitary and/or hypothalamic gland); (m) inovulation; and (n) infertility; in a subject in need thereof.

### DETAILED DESCRIPTION OF THE INVENTION

The present invention is directed to compounds of formula (I)

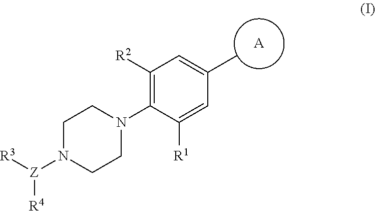

wherein R¹, R²,

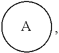

Z, R³ and R⁴ are as herein defined and enantiomers and pharmaceutically acceptable salts thereof. The compounds of the present invention are modulators of the NPY Y₂ receptor, useful in the treatment of disorders and conditions including, but not limited to anxiolytic disorders, depression; pain, injured mammalian nerve tissue; conditions responsive to treatment with a neurotrophic factor; neurological disorders; bone loss; cardiovascular diseases; sleep-wake state disorders, substance abuse and addiction related disorders; obesity; obesity-related disorders, disorders responsive to modulation of endocrine function, inovulation and infertility.

The compounds of formula (I) are preferably, useful for the treatment of disorders or conditions mediated by the NPY Y₂ receptor, selected from the group consisting of substance abuse (more preferably alcohol abuse), anxiolytic disorders (more preferably anxiety), bone loss, obesity and obesity-related disorders. More preferably, the compounds of formula (I) are useful in the treatment of anxiety and alcohol abuse.

In an embodiment of the present invention, R¹ and R² are each independently selected from the group consisting of hydrogen, halogen, C₁₋₄alkyl, —C₁₋₄alkyl-OH, —C₁₋₄alkyl-O—C₁₋₄alkyl, cyano, nitro, —NR^{A}R^{B}, —CH₂—NR^{A}R^{B}, —C(O)—NR^{A}R^{B} and —C(O)H; wherein R^{A} and R^{B} are each independently selected from the group consisting of hydrogen and C₁₋₄alkyl; provided that at least one or R¹ or R² is other than hydrogen.

In an embodiment of the present invention, R¹ is selected from the group consisting of halogen, cyano, C₁₋₄alkyl-OH, —CH₂—NR^{A}R^{B}, —C₁₋₂alkyl-O—C₁₋₂alkyl-, —C(O)H and —C(O)—NR^{A}R^{B}; wherein R^{A} and R^{B} are each independently selected from the group consisting of hydrogen and C₁₋₂alkyl. In another embodiment of the present invention, R¹ is selected from the group consisting of fluoro, bromo, cyano, hydroxy-methyl-, methylamino-methyl-, dimethylamino-methyl-, methoxy-methyl-, —C(O)H and —C(O)—NH₂. In another embodiment of the present invention, R¹ is selected from the group consisting of fluoro, bromo, cyano, hydroxy-methyl-, methoxy-methyl- and —C(O)H.

In another embodiment of the present invention, R¹ is selected from the group consisting of fluoro, bromo, cyano, hydroxy-methyl- and —C(O)H. In another embodiment of the present invention, R¹ is selected from the group consisting of fluoro, bromo, cyano and —C(O)H. In another embodiment of the present invention, R¹ is selected from the group consisting of fluoro, bromo and cyano. In another embodiment of the present invention, R¹ is selected from the group consisting of fluoro and cyano.

In an embodiment of the present invention, R² is selected from the group consisting of hydrogen and halogen. In another embodiment of the present invention, and R² is selected from the group consisting of hydrogen and fluoro. In another embodiment of the present invention, and R² is hydrogen.

In an embodiment of the present invention,

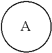

is selected from the group consisting of aryl, heteroaryl and heterocycloalkyl; wherein the aryl, heteroaryl or heterocycloalkyl is optionally substituted with one or more substituents independently selected from the group consisting of halogen, C₁₋₆alkyl, halogenated C₁₋₄alkyl, C₁₋₄alkoxy, —S—C₁₋₄alkyl, cyano, oxo, C₃₋₈cycloalkyl, phenyl, —C(O)O—C₁₋₄alkyl, —C(O)—NR^{C}R^{D} and —C(NR^{C}R^{D})═N—OH; wherein R^{C} and R^{D} are each independently selected from the group consisting of hydrogen and C₁₋₄alkyl. In another embodiment of the present invention,

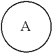

is selected from the group consisting of aryl, heteroaryl and heterocycloalkyl; wherein the aryl, heteroaryl or heterocycloalkyl is optionally substituted with one to two substituents independently selected from the group consisting of halogen, C₁₋₆alkyl, halogenated C₁₋₄alkyl, C₁₋₄alkoxy, cyano, oxo, —S—C₁₋₄alkyl, —C(O)OH, —C(O)O—C₁₋₄alkyl, —C(O)—NH₂, —C(NH₂)═N—OH, C₃₋₈cycloalkyl and phenyl.

In another embodiment of the present invention,

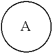

is selected from the group consisting of phenyl, 4-fluorophenyl, 3-chlorophenyl, 4-chlorophenyl, 2-methylphenyl, 3-methylphenyl, 4-methylphenyl, 2-methoxyphenyl, 4-methoxyphenyl, 2-cyanophenyl, 4-(1-methyl-pyrazolyl), 4-(3-methyl-pyrazolyl), 5-(1-phenyl-3-methyl-pyrazolyl), 3-(1-isopropyl-5-oxo-4,5-dihydro-pyrazolyl), 2-pyrrolyl, 2-(1-t-butoxycarbonyl-pyrrolyl), 2-pyridyl, 3-pyridyl, 2-oxazolyl, 2-(4,5-dihydro-4-methoxycarbonyl-oxazolyl), 2-(4-methoxycarbonyl-oxazolyl), 2-(5-ethyl-oxazolyl), 2-(5-methyl-[1,3,4]-oxadiazolyl), 2-(5-ethyl-[1,3,4]-oxadiazolyl), 2-(5-isopropyl-[1,3,4]-oxadiazolyl), 3-[1,2,4]-oxadiazolyl, 3-(5-methyl-[1,2,4]-oxadiazolyl), 3-(5-n-butyl-[1,2,4]-oxadiazolyl), 3-(5-isopropyl-[1,2,4]-oxadiazolyl), 3-(5-t-butyl-[1,2,4]-oxadiazolyl), 3-(5-(3-n-pentyl)[1,2,4]-oxadiazolyl), 3-(5-fluoromethyl-[1,2,4]-oxadiazolyl), 3-(5-trifluoromethyl-[1,2,4]-oxadiazolyl), 3-(5-cyano-[1,2,4]-oxadiazolyl), 3-(5-methylthio-[1,2,4]-oxadiazolyl), 3-(5-carboxy-[1,2,4]-oxadiazolyl), 3-(5-ethoxycarbonyl[1,2,4]-oxadiazolyl), 3-(5-(C(NH₂)═N(OH))[1,2,4]-oxadiazolyl), 3-(5-(aminocarbonyl)-[1,2,4]-oxadiazolyl), 3-(4-methyl-5-oxo-[1,2,4]-oxadiazolyl), 3-(5-cyclopropyl[1,2,4]-oxadiazolyl), 3-(5-cyclobutyl-[1,2,4]-oxadiazolyl), 3-(5-(1-(1-(S)methyl-n-propyl)[1,2,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(1,5-dimethyl-[1,2,4]-triazolyl), 3-(1-methyl-5-isopropyl-[1,2,4]-triazolyl), 5-(3-isopropyl-[1,2,4]-triazolyl and 5-(1-methyl-3-isopropyl-[1,2,4]-triazolyl).

In another embodiment of the present invention,

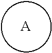

is selected from the group consisting of phenyl, 4-fluorophenyl, 3-chlorophenyl, 4-chlorophenyl, 2-methylphenyl, 3-methylphenyl, 4-methylphenyl, 2-methoxyphenyl, 4-methoxyphenyl, 2-cyanophenyl, 4-(1-methyl-pyrazolyl), 4-(3-methyl-pyrazolyl), 5-(1-phenyl-3-methyl-pyrazolyl), 3-(1-isopropyl-5-oxo-4,5-dihydro-pyrazolyl), 2-pyrrolyl, 2-(1-t-butoxycarbonyl-pyrrolyl), 2-pyridyl, 3-pyridyl, 2-oxazolyl, 2-(4,5-dihydro-4-methoxycarbonyl-oxazolyl), 2-(4-methoxycarbonyl-oxazolyl), 2-(5-ethyl-oxazolyl), 2-(5-methyl-[1,3,4]-oxadiazolyl), 2-(5-ethyl-[1,3,4]-oxadiazolyl), 2-(5-isopropyl-[1,3,4]-oxadiazolyl), 3-[1,2,4]-oxadiazolyl, 3-(5-methyl-[1,2,4]-oxadiazolyl), 3-(5-n-butyl-[1,2,4]-oxadiazolyl), 3-(5-isopropyl-[1,2,4]-oxadiazolyl), 3-(5-t-butyl-[1,2,4]-oxadiazolyl), 3-(5-(3-n-pentyl)[1,2,4]-oxadiazolyl), 3-(5-fluoromethyl-[1,2,4]-oxadiazolyl), 3-(5-trifluoromethyl-[1,2,4]-oxadiazolyl), 3-(5-cyano-[1,2,4]-oxadiazolyl), 3-(5-methylthio-[1,2,4]-oxadiazolyl), 3-(5-carboxy-[1,2,4]-oxadiazolyl), 3-(5-ethoxycarbonyl[1,2,4]-oxadiazolyl), 3-(5-(C(NH₂)═N(OH))-[1,2,4]-oxadiazolyl), 3-(5-(aminocarbonyl)[1,2,4]-oxadiazolyl), 3-(4-methyl-5-oxo-[1,2,4]-oxadiazolyl), 3-(5-cyclopropyl[1,2,4]-oxadiazolyl), 3-(5-cyclobutyl-[1,2,4]-oxadiazolyl), 3-(5-(1-(1-(S)methyl-n-propyl)[1,2,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(1,5-dimethyl-[1,2,4]-triazolyl), 3-(1-methyl-5-isopropyl-[1,2,4]-triazolyl), 5-(3-isopropyl-[1,2,4]-triazolyl, 5-(1-methyl-3-isopropyl-[1,2,4]-triazolyl) and 5-(3-methyl-[1,2,4]-thiadiazolyl).

In another embodiment of the present invention,

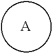

is selected from the group consisting of phenyl, 3-chlorophenyl, 4-chlorophenyl, 3-methylphenyl, 4-methylphenyl, 2-methoxyphenyl, 4-methoxyphenyl, 4-(1-methyl-pyrazolyl), 4-(3-methyl-pyrazolyl), 5-(1-phenyl-3-methyl-pyrazolyl), 3-(1-isopropyl-5-oxo-4,5-dihydro-pyrazolyl), 2-pyrrolyl, 2-pyridyl, 2-(5-ethyl-oxazolyl), 2-(5-methyl-[1,3,4]-oxadiazolyl), 2-(5-ethyl-[1,3,4]-oxadiazolyl), 2-(5-isopropyl-[1,3,4]-oxadiazolyl), 3-(5-methyl-[1,2,4]-oxadiazolyl), 3-(5-n-butyl-[1,2,4]-oxadiazolyl), 3-(5-isopropyl-[1,2,4]-oxadiazolyl), 3-(5-t-butyl-[1,2,4]-oxadiazolyl), 3-(5-(3-n-pentyl)-[1,2,4]-oxadiazolyl), 3-(5-fluoromethyl-[1,2,4]-oxadiazolyl), 3-(5-trifluoromethyl-[1,2,4]-oxadiazolyl), 3-(5-methylthio-[1,2,4]-oxadiazolyl), 3-(5-cyclopropyl-[1,2,4]-oxadiazolyl), 3-(5-cyclobutyl-[1,2,4]-oxadiazolyl), 3-(5-(1-(1-(S)-methyl-n-propyl)-[1,2,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl) and 5-(3-isopropyl-[1,2,4]-triazolyl.

In another embodiment of the present invention,

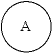

is selected from the group consisting of phenyl, 3-chlorophenyl, 4-chlorophenyl, 3-methylphenyl, 4-methylphenyl, 2-methoxyphenyl, 4-methoxyphenyl, 4-(1-methyl-pyrazolyl), 4-(3-methyl-pyrazolyl), 5-(1-phenyl-3-methyl-pyrazolyl), 3-(1-isopropyl-5-oxo-4,5-dihydro-pyrazolyl), 2-pyrrolyl, 2-pyridyl, 2-(5-ethyl-oxazolyl), 2-(5-methyl-[1,3,4]-oxadiazolyl), 2-(5-ethyl-[1,3,4]-oxadiazolyl), 2-(5-isopropyl-[1,3,4]-oxadiazolyl), 3-(5-methyl-[1,2,4]-oxadiazolyl), 3-(5-n-butyl-[1,2,4]-oxadiazolyl), 3-(5-isopropyl-[1,2,4]-oxadiazolyl), 3-(5-t-butyl-[1,2,4]-oxadiazolyl), 3-(5-(3-n-pentyl)-[1,2,4]-oxadiazolyl), 3-(5-fluoromethyl-[1,2,4]-oxadiazolyl), 3-(5-trifluoromethyl-[1,2,4]-oxadiazolyl), 3-(5-methylthio-[1,2,4]-oxadiazolyl), 3-(5-cyclopropyl-[1,2,4]-oxadiazolyl), 3-(5-cyclobutyl-[1,2,4]-oxadiazolyl), 3-(5-(1-(1-(S)-methyl-n-propyl)-[1,2,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 5-(3-isopropyl-[1,2,4]-triazolyl and 5-(3-methyl-[1,2,4]-thiadiazolyl)

In another embodiment of the present invention,

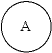

is selected from the group consisting of phenyl, 3-methylphenyl, 2-methoxyphenyl, 5-(1-phenyl-3-methyl-pyrazolyl), 3-(1-isopropyl-5-oxo-4,5-dihydro-pyrazolyl), 2-(5-ethyl-oxazolyl), 2-(5-methyl-[1,3,4]-oxadiazolyl), 2-(5-ethyl-[1,3,4]-oxadiazolyl), 2-(5-isopropyl-[1,3,4]-oxadiazolyl), 3-(5-methyl-[1,2,4]-oxadiazolyl), 3-(5-isopropyl-[1,2,4]-oxadiazolyl), 3-(5-(3-n-pentyl)[1,2,4]-oxadiazolyl), 3-(5-fluoromethyl-[1,2,4]-oxadiazolyl), 3-(5-trifluoromethyl-[1,2,4]-oxadiazolyl), 3-(5-cyclopropyl-[1,2,4]-oxadiazolyl), 5-(3-methyl-[1,2,4]-oxadiazolyl), 5-(3-ethyl-[1,2,4]-oxadiazolyl) and 5-(3-isopropyl-[1,2,4]-oxadiazolyl.

In another embodiment of the present invention,

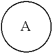

is selected from the group consisting of phenyl, 3-methylphenyl, 2-methoxyphenyl, 5-(1-phenyl-3-methyl-pyrazolyl), 3-(1-isopropyl-5-oxo-4,5-dihydro-pyrazolyl), 2-(5-ethyl-oxazolyl), 2-(5-methyl-[1,3,4]-oxadiazolyl), 2-(5-ethyl-[1,3,4]-oxadiazolyl), 2-(5-isopropyl-[1,3,4]-oxadiazolyl), 3-(5-methyl-[1,2,4]-oxadiazolyl), 3-(5-isopropyl-[1,2,4]-oxadiazolyl), 3-(5-(3-n-pentyl)[1,2,4]-oxadiazolyl), 3-(5-fluoromethyl-[1,2,4]-oxadiazolyl), 3-(5-trifluoromethyl-[1,2,4]-oxadiazolyl), 3-(5-cyclopropyl-[1,2,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl and 5-(3-methyl-[1,2,4]-thiadiazolyl)

In another embodiment of the present invention,

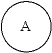

is selected from the group consisting of 2-methoxyphenyl, 5-(1-phenyl-3-methyl-pyrazolyl), 3-(5-methyl-[1,2,4]-oxadiazolyl), 3-(5-isopropyl-[1,2,4]-oxadiazolyl), 3-(5-fluoromethyl-[1,2,4]-oxadiazolyl), 3-(5-trifluoromethyl-[1,2,4]-oxadiazolyl), 5-(3-methyl-[1,2,4]-oxadiazolyl), 5-(3-ethyl-[1,2,4]-oxadiazolyl) and 5-(3-isopropyl-[1,2,4]-oxadiazolyl.

In another embodiment of the present invention,

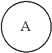

is selected from the group consisting of 2-methoxyphenyl, 5-(1-phenyl-3-methyl-pyrazolyl), 3-(5-methyl-[1,2,4]-oxadiazolyl), 3-(5-isopropyl-[1,2,4]-oxadiazolyl), 3-(5-fluoromethyl-[1,2,4]-oxadiazolyl), 3-(5-trifluoromethyl-[1,2,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl and 5-(3-methyl-[1,2,4]-thiadiazolyl)

In another embodiment of the present invention,

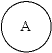

is selected from the group consisting of 3-(5-methyl-[1,2,4]-oxadiazolyl), 3-(5-isopropyl-[1,2,4]-oxadiazolyl), 5-(3-methyl-[1,2,4]-oxadiazolyl), 5-(3-ethyl-[1,2,4]-oxadiazolyl) and 5-(3-isopropyl-[1,2,4]-oxadiazolyl).

In another embodiment of the present invention,

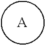

is selected from the group consisting of 2-methoxyphenyl, 5-(3-methyl-[1,2,4]-oxadiazolyl), 5-(3-ethyl-[1,2,4]-oxadiazolyl) and 5-(3-isopropyl-[1,2,4]-oxadiazolyl.

In an embodiment of the present invention, Z is selected from the group consisting of CH and CR⁰; wherein R⁰ is selected from the group consisting of —C₁₋₄-alkyl. In another embodiment of the present invention, Z is selected from the group consisting of CH and CR⁰; wherein R⁰ is selected from the group consisting of C₁₋₂alkyl. In another embodiment of the present invention, Z is selected from the group consisting of CH and C(CH₃). In another embodiment of the present invention, Z is CH.

In an embodiment of the present invention, R³ is selected from the group consisting of C₃₋₈cycloalkyl, aryl and 5 to 6 membered heteroaryl; wherein the aryl or heteroaryl, whether alone or as part of a substituent group is optionally substituted with one or more substituents independently selected from the group consisting of halogen, C₁₋₄alkyl, halogenated C₁₋₄alkyl, C₁₋₄alkoxy, halogenated C₁₋₄alkoxy, cyano, NR^{E}R^{F} and —C(O)—NR^{E}R^{F}; wherein R^{E} and R^{F} are each independently selected from the group consisting of hydrogen and C₁₋₄alkyl. In another embodiment of the present invention, R³ is selected from the group consisting of aryl and 5 to 6 membered heteroaryl; wherein the aryl is optionally substituted with one to two substituents independently selected from the group consisting of halogen and C₁₋₄alkoxy.

In another embodiment of the present invention, R³ is selected from the group consisting of phenyl, 3-fluoro-phenyl, 4-fluoro-phenyl, 2-bromo-5-fluoro-phenyl, 2-methoxy-phenyl, 4-methoxy-phenyl and 2-thienyl. In another embodiment of the present invention, R³ is selected from the group consisting of phenyl, 3-fluoro-phenyl, 4-fluoro-phenyl, 2-bromo-5-fluoro-phenyl, 2-methoxy-phenyl, 4-methoxy-phenyl and 2-thienyl. In another embodiment of the present invention, R³ is selected from the group consisting of phenyl, 3-fluoro-phenyl, 4-fluoro-phenyl, 2-methoxy-phenyl, 4-methoxy-phenyl and 2-thienyl. In another embodiment of the present invention, R³ is selected from the group consisting of phenyl, 3-fluoro-phenyl, 4-fluoro-phenyl, 4-methoxy-phenyl and 2-thienyl. In another embodiment of the present invention, R³ is selected from the group consisting of phenyl, 3-fluoro-phenyl, 4-fluoro-phenyl, 4-methoxy-phenyl and 2-thienyl. In another embodiment of the present invention, R³ is selected from the group consisting of phenyl and 3-fluorophenyl.

In an embodiment of the present invention, R⁴ is selected from the group consisting of cyano, C₁₋₈alkyl, —C(O)—NR^{G}R^{H}, —C(O)—C₁₋₄alkyl, —C(O)—O—C₁₋₄alkyl; wherein R^{G} and R^{H} are each independently selected from the group consisting of hydrogen and C₁₋₄alkyl; alternatively, R^{G} and R^{H} are taken together with the nitrogen atom to which they are bound to form a 4 to 8 membered saturated ring structure; wherein the 4 to 8 membered saturated ring structure is optionally substituted with one or more halogen. In another embodiment of the present invention, R⁴ is selected from the group consisting of —C(O)—NR^{G}R^{H}, —C(O)—C₁₋₄alkyl and —C₁₋₄alkyl-O—C₁₋₄alkyl; wherein R^{G} and R^{H} are each independently selected from the group consisting of hydrogen and C₁₋₄alkyl; alternatively, alternatively, R^{G} and R^{H} are taken together with the nitrogen atom to which they are bound to form a 4 to 8 membered saturated ring structure; wherein the 4 to 8 membered saturated ring structure is optionally substituted with one to two halogen.

In another embodiment of the present invention, R⁴ is selected from the group consisting of ethylamino-carbonyl-, diethylamino-carbonyl-, N-methyl-N-ethyl-amino-carbonyl, 1-pyrrolidinyl-carbonyl-, 1-(4,4-difluoro-piperidinyl)-carbonyl-, methyl-carbonyl-, methoxy-carbonyl-, ethoxy-carbonyl- and t-butoxy-carbonyl-. In another embodiment of the present invention, R⁴ is selected from the group consisting of ethylamino-carbonyl-, diethylamino-carbonyl-, N-methyl-N-ethyl-amino-carbonyl, 1-pyrrolidinyl-carbonyl-, 1-(4,4-difluoro-piperidinyl)-carbonyl-, methoxy-carbonyl-, ethoxy-carbonyl- and t-butoxy-carbonyl-. In another embodiment of the present invention, R⁴ is selected from the group consisting of ethylamino-carbonyl-, diethylamino-carbonyl-, N-methyl-N-ethyl-amino-carbonyl, 1-pyrrolidinyl-carbonyl-, 1-(4,4-difluoro-piperidinyl)-carbonyl-, methoxy-carbonyl- and ethoxy-carbonyl-. In another embodiment of the present invention, R⁴ is selected from the group consisting of diethylamino-carbonyl-, N-methyl-N-ethyl-amino-carbonyl and 1-pyrrolidinyl-carbonyl-. In another embodiment of the present invention, R⁴ is selected from the group consisting of ethylamino-carbonyl-, diethylamino-carbonyl-, N-methyl-N-ethyl-amino-carbonyl and 1-pyrrolidinyl-carbonyl-. In another embodiment of the present invention, R⁴ is selected from the group consisting of diethylamino-carbonyl- and N-methyl-N-ethyl-amino-carbonyl-. In another embodiment of the present invention, R⁴ is selected from the group consisting of ethylamino-carbonyl, diethylamino-carbonyl- and N-methyl-N-ethyl-amino-carbonyl-. In another embodiment of the present invention, R⁴ is diethylaminocarbonyl.

In an embodiment of the present invention R⁴ is other than —C(O)NR^{G}R^{H} or —C(O)O—C₁₋₄alkyl. In another embodiment of the present invention,

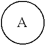

is other than substituted or unsubstituted furo[2,3-b]pyridinyl, 1,3-dihydro-imidazolyl, 4-dibenzothienyl or benzoimidazolyl; preferably

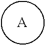

is other than 2-(4-chloro-3-phenyl-furo[2,3-b]pyridinyl), 1-(3-isopropyl-1,3-dihydro-imidazol-2-one), 4-dibenzothienyl, 2-(7-aminocarbonyl-1H-benzoimidazolyl), or 4-cyanophenyl.

Additional embodiments of the present invention, include those wherein the substituents selected for one or more of the variables defined herein (e.g. R¹, R²,

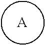

L¹, R⁵, Z, R³, R⁴, etc.) are independently selected to be any individual substituent or any subset of substituents selected from the complete list as defined herein. In another embodiment of the present invention is any single compound or subset of compounds selected from the representative compounds listed in Tables 1-2 below.

Representative compounds of formula (I) of the present invention are as listed in Tables 1 and 2 below. Unless otherwise noted, wherein a stereogenic center is present in the listed compound, the compound was prepared as a mixture of stereo-configurations. Where a stereogenic center is present, the (S) and (R) designations are intended to indicate that the exact stereo-configuration of the center has not been determined.

TABLE 1
Representative Compounds of Formula (I)

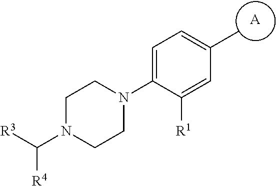

| Cmpd. No | R¹ | 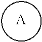 | R³ | R⁴ |
|---|---|---|---|---|
| 3 | F | 5-(3-ethyl-[1,2,4]- | phenyl | diethyl-amino- |
| | | oxadiazolyl | | carbonyl- |
| 67 | F | 3-(5-methyl-[1,2,4]- | phenyl | diethyl-amino- |
| | | oxadiazolyl) | | carbonyl- |
| 68 | F | 3-(5-n-butyl-[1,2,4]- | phenyl | diethyl-amino- |
| | | oxadiazolyl) | | carbonyl- |
| 69 | F | 3-(5-isopropyl- | phenyl | diethyl-amino- |
| | | [1,2,4]-oxadiazolyl) | | carbonyl- |
| 70 | F | 3-(5-(3-n-pentyl)- | phenyl | diethyl-amino- |
| | | [1,2,4]-oxadiazolyl) | | carbonyl- |
| 71 | F | 5-(3-ethyl-[1,2,4]- | phenyl | diethyl-amino- |
| | | oxadiazolyl) | | carbonyl- |
| 75 | F | 3-(5-t-butyl)-[1,2,4]- | phenyl | diethyl-amino- |
| | | oxazdiazolyl) | | carbonyl- |
| 79 | F | 3-(5-t-butyl-[1,2,4]- | phenyl | diethyl-amino- |
| | | oxadiazolyl) | | carbonyl- |
| 80 | F | 5-(3-isopropyl)- | phenyl | diethyl-amino- |
| | | [1,2,4]-oxadiazolyl) | | carbonyl- |
| 88 | F | 3-(5-cyclopropyl- | phenyl | diethyl-amino- |
| | | [1,2,4]-oxadiazolyl) | | carbonyl- |
| 89 | F | 3-(5-cyclobutyl- | phenyl | diethyl-amino- |
| | | [1,2,4]-oxadiazolyl) | | carbonyl- |
| 97 | F | 3-(5-(1-(1-(S)- | phenyl | diethyl-amino- |
| | | methyl-n-propyl)- | | carbonyl- |
| | | [1,2,4]-oxadiazolyl | | |
| 105 | F | 3-(5-fluoromethyl- | phenyl | diethyl-amino- |
| | | [1,2,4]-oxadiazolyl) | | carbonyl- |
| 118 | F | 3-(5-trifluoro-methyl- | phenyl | diethyl-amino- |
| | | [1,2,4]-oxadiazolyl) | | carbonyl- |
| 119 | F | 5-(3-methyl-[1,2,4]- | phenyl | diethyl-amino- |
| | | oxadiazolyl) | | carbonyl- |
| 124 | F | 2-(4,5-dihydro-4- | phenyl | diethyl-amino- |
| | | (methoxy-carbonyl)- | | carbonyl- |
| | | oxazolyl) | | |
| 129 | F | 2-(4-methoxy- | phenyl | diethyl-amino- |
| | | carbonyl-oxazolyl) | | carbonyl- |
| 130 | F | 3-(5-ethoxy- | phenyl | diethyl-amino- |
| | | carbonyl-[1,2,4]- | | carbonyl- |
| | | oxadiazolyl) | | |
| 133 | F | 2-(5-ethyl-oxazolyl) | phenyl | diethyl-amino- |
| | | | | carbonyl- |
| 144 | F | 3-(5-(amino- | phenyl | diethyl-amino- |
| | | carbonyl)-[1,2,4]- | | carbonyl- |
| | | oxadiazolyl) | | |
| 147 | F | 3-(5-cyano-[1,2,4]- | phenyl | diethyl-amino- |
| | | oxadiazolyl) | | carbonyl- |
| 149 | F | 3-(5- | phenyl | diethyl-amino- |
| | | (C(NH₂)═N(OH))- | | carbonyl- |
| | | [1,2,4]-oxadiazolyl) | | |
| 150 | F | 2-(5-methyl-[1,3,4]- | phenyl | diethyl-amino- |
| | | oxadiazolyl) | | carbonyl- |
| 151 | F | 3-(5-carboxy-[1,2,4]- | phenyl | diethyl-amino- |
| | | oxadiazolyl) | | carbonyl- |
| 152 | F | 2-(5-ethyl-[1,3,4]- | phenyl | diethyl-amino- |
| | | oxadiazolyl) | | carbonyl- |
| 154 | F | 2-(5-isopropyl- | phenyl | diethyl-amino- |
| | | [1,3,4]-oxadiazolyl) | | carbonyl- |
| 158 | F | 3-[1,2,4]-oxadiazolyl | phenyl | diethyl-amino- |
| | | | | carbonyl- |
| 176 | F | 3-(4-methyl-5-oxo- | phenyl | diethyl-amino- |
| | | [1,2,4]oxadiazolyl) | | carbonyl- |
| 177 | F | 3-(5-methylthio- | phenyl | diethyl-amino- |
| | | [1,2,4]-oxadiazolyl) | | carbonyl- |
| 178 | F | 2-oxazolyl | phenyl | diethyl-amino- |
| | | | | carbonyl- |
| 180 | F | 5-(3-isopropyl- | phenyl | diethyl-amino- |
| | | [1,2,4]-oxadiazolyl) | | carbonyl- |
| 181 | F | 5-(3-ethyl-[1,2,4]- | phenyl | diethyl-amino- |
| | | oxadiazolyl) | | carbonyl- |
| 182 | F | 5-(3-methyl-[1,2,4]- | phenyl | diethyl-amino- |
| | | oxadiazolyl) | | carbonyl- |
| 183 | F | 5-(3-methyl-[1,2,4]- | phenyl | methoxy- |
| | | oxadiazolyl) | | carbonyl- |
| 184 | F | 5-(3-isopropyl- | phenyl | N-methyl, N- |
| | | [1,2,4]-oxadiazolyl) | | ethyl amino- |
| | | | | carbonyl |
| 185 | F | 5-(3-isopropyl- | phenyl | 1-pyrrolidinyl- |
| | | [1,2,4]-oxadiazolyl) | | carbonyl |
| 186 | F | 5-(1-phenyl-3- | phenyl | diethyl-amino- |
| | | methyl-pyrazolyl) | | carbonyl |
| 187 | F | 2-(1-t-butoxycarbonyl- | phenyl | diethyl-amino- |
| | | pyrrolyl) | | carbonyl |
| 188 | F | 2-pyrrolyl | phenyl | diethyl-amino- |
| | | | | carbonyl |
| 189 | F | 5-(3-methyl-[1,2,4]- | phenyl | 1-(4,4- |
| | | oxadiazolyl) | | difluoro- |
| | | | | piperidinyl- |
| | | | | carbonyl |
| 190 | F | 5-(3-methyl-[1,2,4]- | phenyl | ethyl-amino- |
| | | oxadiazolyl) | | carbonyl |
| 191 | F | 3-(1-isopropyl-5- | phenyl | diethyl-amino- |
| | | oxo-4,5-dihydro- | | carbonyl |
| | | pyrazolyl) | | |
| 192 | F | 5-(3-methyl-[1,2,4]- | phenyl | ethoxy- |
| | | oxadiazolyl) | | carbonyl- |
| 193 | F | phenyl | phenyl | diethyl-amino- |
| | | | | carbonyl |
| 194 | F | 3-chloro-phenyl | phenyl | diethyl-amino- |
| | | | | carbonyl |
| 195 | F | 4-(1-methyl-pyrazolyl) | phenyl | diethyl-amino- |
| | | | | carbonyl |
| 196 | F | 4-chloro-phenyl | phenyl | diethyl-amino- |
| | | | | carbonyl |
| 197 | F | 5-(3-ethyl-[1,2,4]- | phenyl | N-methyl, N- |
| | | oxadiazolyl) | | ethyl-amino- |
| | | | | carbonyl |
| 198 | F | 5-(3-isopropyl- | phenyl | methyl- |
| | | [1,2,4]-oxadiazolyl) | | carbonyl |
| 199 | F | 5-(3-methyl-[1,2,4]- | n-butyl | diethyl-amino- |
| | | oxadiazolyl) | | carbonyl |
| 200 | F | 4-(3-methyl- | phenyl | diethyl-amino- |
| | | pyrazolyl) | | carbonyl |
| 201 | F | 2-pyridyl | phenyl | diethyl-amino- |
| | | | | carbonyl |
| 202 | F | 3-pyridyl | phenyl | diethyl-amino- |
| | | | | carbonyl |
| 203 | Br | 5-(3-methyl-[1,2,4]- | phenyl | diethyl-amino- |
| | | oxadiazolyl) | | carbonyl |
| 204 | F | 4-methyl-phenyl | phenyl | diethyl-amino- |
| | | | | carbonyl |
| 205 | F | 4-methoxy-phenyl | phenyl | diethyl-amino- |
| | | | | carbonyl |
| 206 | F | 3-methyl-phenyl | phenyl | diethyl-amino- |
| | | | | carbonyl |
| 207 | F | 4-fluoro-phenyl | phenyl | diethyl-amino- |
| | | | | carbonyl |
| 208 | F | 5-(3-methyl-[1,2,4]- | phenyl | diethyl-amino- |
| | | triazolyl) | | carbonyl |
| 209 | F | 5-(3-methyl-[1,2,4]- | 2-bromo-5- | diethyl-amino- |
| | | oxadiazolyl) | fluoro- | carbonyl |
| | | | phenyl | |
| 210 | F | 5-(3-methyl-[1,2,4]- | 4-fluoro- | diethyl-amino- |
| | | oxadiazolyl) | phenyl | carbonyl |
| 211 | F | 5-(3-methyl-[1,2,4]- | 3-fluoro- | diethyl-amino- |
| | | oxadiazolyl) | phenyl | carbonyl |
| 212 | F | 5-(3-isopropyl- | phenyl | diethyl-amino- |
| | | [1,2,4]-triazolyl) | | carbonyl |
| 213 | F | 5-(3-isopropyl- | phenyl | methoxy- |
| | | [1,2,4]-oxadiazolyl) | | carbonyl |
| 214 | F | 2-methoxy-phenyl | phenyl | diethyl-amino- |
| | | | | carbonyl |
| 215 | F | 3-(1,5-dimethyl- | phenyl | diethyl-amino- |
| | | [1,2,4]-triazolyl) | | carbonyl |
| 216 | F | 3-(1-methyl-5- | phenyl | diethyl-amino- |
| | | isopropyl-[1,2,4]- | | carbonyl |
| | | triazolyl) | | |
| 217 | cyano | 5-(3-methyl-[1,2,4]- | phenyl | diethyl-amino- |
| | | oxadiazolyl) | | carbonyl |
| 218 | cyano | 5-(3-isopropyl- | phenyl | diethyl-amino- |
| | | [1,2,4]-oxadiazolyl) | | carbonyl |
| 219 | F | 5-(3-methyl-[1,2,4]- | phenyl | t-butoxy- |
| | | oxadiazolyl) | | carbonyl |
| 220 | F | 5-(1-methyl-3- | phenyl | diethyl-amino- |
| | | isopropyl-[1,2,4]- | | carbonyl |
| | | triazolyl) | | |
| 221 | amino- | 5-(3-isopropyl- | phenyl | diethyl-amino- |
| | carbonyl | [1,2,4]-oxadiazolyl) | | carbonyl |
| 250 | cyano | 5-(3-methyl-[1,2,4]- | 4-methoxy- | diethyl-amino- |
| | | oxadiazolyl) | phenyl | carbonyl |
| 251 | cyano | 5-(3-methyl-[1,2,4]- | 2-methoxy- | diethyl-amino- |
| | | oxadiazolyl) | phenyl | carbonyl |
| 252 | cyano | 5-(3-methyl-[1,2,4]- | 2-thienyl | diethyl-amino- |
| | | oxadiazolyl) | | carbonyl |
| 253 | fluoro | 2-cyano-phenyl | phenyl | diethyl-amino- |
| | | | | carbonyl |
| 254 | fluoro | 2-methyl-phenyl | phenyl | diethyl-amino- |
| | | | | carbonyl |
| 255 | cyano | 2-methoxy-phenyl | phenyl | diethyl-amino- |
| | | | | carbonyl |
| 256 | —C(O)H | 5-(3-isopropyl- | phenyl | diethyl-amino- |
| | | [1,2,4]-oxadiazolyl) | | carbonyl |
| 257 | hydroxy- | 5-(3-isopropyl- | phenyl | diethyl-amino- |
| | methyl- | [1,2,4]-oxadiazolyl) | | carbonyl |
| 258 | methyl- | 5-(3-isopropyl- | phenyl | diethyl-amino- |
| | amino- | [1,2,4]-oxadiazolyl) | | carbonyl |
| | methyl- | | | |
| 259 | dimethyl- | 5-(3-isopropyl- | phenyl | diethyl-amino- |
| | amino- | [1,2,4]-oxadiazolyl) | | carbonyl |
| | methyl- | | | |
| 260 | methoxy- | 5-(3-isopropyl- | phenyl | diethyl-amino- |
| | methyl- | [1,2,4]-oxadiazolyl) | | carbonyl |
| 261 | cyano | 5-(3-methyl-[1,2,4]- | phenyl | diethyl-amino- |
| | | oxadiazolyl) | | carbonyl |
| 262 | cyano | 5-(3-methyl-[1,2,4]- | 4-fluoro- | diethyl-amino- |
| | | oxadiazolyl) | phenyl | carbonyl |
| 263 | cyano | 5-(3-methyl-[1,2,4]- | 3-fluoro- | diethyl-amino- |
| | | oxadiazolyl) | phenyl | carbonyl |
| 270 | cyano | 5-(3-isopropyl- | phenyl | ethylamino- |
| | | [1,2,4]-oxadiazolyl) | | carbonyl- |
| 271 | fluoro | 5-(3-methyl-[1,2,4]- | phenyl | diethylamino- |
| | | thiadiazolyl) | | carbonyl- |

Additional representative compounds of formula (I) are as listed in Table 2 below.

TABLE 2
Representative Compounds of Formula (I)

| Cmpd. | |
|---|---|
| No. | Structure |
| 226 | 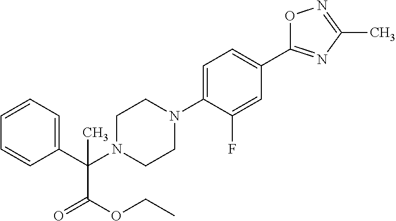 |

As used herein, “halogen” shall mean chlorine, bromine, fluorine and iodine.

As used herein, the term “alkyl” whether used alone or as part of a substituent group, include straight and branched chains. For example, alkyl radicals include methyl, ethyl, propyl, isopropyl, butyl, isobutyl, sec-butyl, t-butyl, pentyl and the like. Unless otherwise noted, “lower” when used with alkyl means a carbon chain composition of 1-4 carbon atoms.

As used herein, unless otherwise noted, the term “halogenated C₁₋₄alkyl” shall mean any C₁₋₄alkyl group as defined above substituted with at least one halogen atom, preferably substituted with a least one fluoro atom. Suitable examples include but are not limited to —CF₃, —CH₂—CF₃, —CF₂—CF₂—CF₂—CF₃, and the like. Similarly, the term “fluorinated C₁₋₄alkyl” shall mean any C₁₋₄alkyl group as defined above substituted with at least one fluoro atom. Suitable examples include but are not limited to —CH₂F, —CF₃, —CH₂—CF₃, —CF₂—CF₂—CF₂—CF₃, and the like.

As used herein, unless otherwise noted, the term “hydroxy substituted alkyl” shall mean alkyl group as defined above substituted with at least one hydroxy group. Preferably, the alkyl group is substituted with one hydroxy group. Preferably, the alkyl group is substituted with a hydroxy group at the terminal carbon. Suitable examples include, but are not limited to, —CH₂(OH), —CH₂—CH₂(OH), —CH₂—CH(OH)—CH₂, and the like.

As used herein, unless otherwise noted, “alkoxy” shall denote an oxygen ether radical of the above described straight or branched chain alkyl groups. For example, methoxy, ethoxy, n-propoxy, sec-butoxy, t-butoxy, n-hexyloxy and the like. Similarly, the term “fluorinated C₁₋₄alkOXY” shall mean any oxygen ether radical as defined above substituted with at least one fluoro atom. Suitable examples include but are not limited to —OCH₂F, —OCF₃, —OCH₂—CF₃, —OCF₂—CF₂—CF₂—CF₃, and the like.

As used herein, unless otherwise noted, “aryl” shall refer to unsubstituted carbocylic aromatic groups such as phenyl, naphthyl, and the like.

As used herein, unless otherwise noted, “aralkyl” shall mean any lower alkyl group substituted with an aryl group such as phenyl, naphthyl and the like. For example, benzyl, phenylethyl, phenylpropyl, naphthylmethyl, and the like.

As used herein, unless otherwise noted, the term “cycloalkyl” shall mean any stable 3-8 membered monocyclic, saturated ring system, for example cyclopropyl, cyclobutyl, cyclopentyl, cyclohexyl, cycloheptyl and cyclooctyl.

As used herein, unless otherwise noted, “heteroaryl” shall denote any five or six membered monocyclic aromatic ring structure containing at least one heteroatom selected from the group consisting of O, N and S, optionally containing one to three additional heteroatoms independently selected from the group consisting of O, N and S; or a nine or ten membered bicyclic aromatic ring structure containing at least one heteroatom selected from the group consisting of O, N and S, optionally containing one to four additional heteroatoms independently selected from the group consisting of O, N and S. Unless otherwise noted, the heteroaryl group may be attached at any heteroatom or carbon atom of the ring such that the result is a stable structure. Examples of suitable heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like. Further, the term “5 to 6 membered heteroaryl” shall mean monocyclic heteroaryl as herein defined, wherein the monocyclic ring structure contains 5 to 6 ring atoms.

As used herein, the term “heterocycloalkyl” shall denote any three to eight, preferably any five to seven, membered monocyclic, saturated or partially unsaturated ring structure containing at least one heteroatom selected from the group consisting of O, N and S, optionally containing one to three additional heteroatoms independently selected from the group consisting of O, N and S; or a nine to ten membered saturated, partially unsaturated or partially aromatic bicyclic ring system containing at least one heteroatom selected from the group consisting of O, N and S, optionally containing one to four additional heteroatoms independently selected from the group consisting of O, N and S. The heterocycloalkyl group may be attached at any heteroatom or carbon atom of the ring such that the result is a stable structure. Examples of suitable heteroaryl groups include, but are not limited to, pyrrolinyl, pyrrolidinyl, dioxalanyl, imidazolinyl, imidazolidinyl, pyrazolinyl, pyrazolidinyl, piperidinyl, dioxanyl, morpholinyl, dithianyl, thiomorpholinyl, piperazinyl, trithianyl, indolinyl, chromenyl, 3,4-methylenedioxyphenyl, 2,3-dihydrobenzofuryl, and the like.

As used herein, unless otherwise noted the term “nitrogen containing heteroaryl” shall mean any heteroaryl as defined above provided that the heteroaryl contains at least one N heteroatom. Similarly, the term “nitrogen containing heterocycloalkyl” shall mean any heterocycloalkyl as defined above provided that the heterocycloalkyl contains at least one N heteroatom.

When a particular group is “substituted” (e.g., alkyl, cycloalkyl, aryl, heteroaryl, heterocycloalkyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

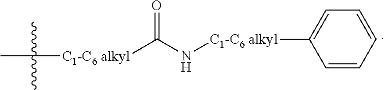

Abbreviations used in the specification, particularly the Schemes and Examples, are as follows:
ACN=Acetonitrile
AcOH=Acetic acid
BINAP=2,2′-Bis(diphenylphosphino)-1,1′-binaphthyl
BOC=t-Butoxycarbonyl
BOC₂O=Boc anhydride
Burgess reagent=Methyl N-(triethylammoniumsulphonyl)carbamate
CBz=Benzyloxycarbonyl
CDI=1,1′Carbonyldiimidazole
^{t}BuOH or t-BuOH=t-Butanol
DBU=1,8-Diazabicyclo[5.4.0]undec-7-ene
DCE=Dichloroethane
DCM=Dichloromethane
DIPEA or DIEA or =Diisopropylethylamine
Hunig's base
DMAP=4-N,N-Dimethylaminopyridine
DMF=N,N-Dimethylformamide
EDTA=Ethylene Diamine Tetraacetic Acid
Et₂O=Diethyl ether
EtOAc=Ethyl acetate
EtOH=Ethanol
HATU=O-(7-Azabenzotriazol-1-yl)-N,N,N″,N″-Tetramethyl Uronium Hexafluorophosphate
HEPES=4-(2-Hydroxyethyl)-1-Piperizine Ethane Sulfonic Acid
HPLC=High Pressure Liquid Chromatography
Lawesson's reagent 2,4-bis(4-methoxyphenyl)-1,3,2,4-dithia-diphosphetane-2,4-disulfide
MeOH=Methanol
NaBH(OAc)₃=Sodium triacetoxyborohydride
NaO^{t}Bu=Sodium t-Butoxide
NaOMe=Sodium Methoxide
NaHMDS=Sodium bis(trimethylsilyl)amide
NMM=4-Methylmorpholine
NMP=N-methyl-2-pyrrolidinone
PBS=Phosphate Buffered Saline
Pd/C=Palladium on Carbon Catalyst
Pd(dppf)Cl₂=dichloro[1,1′-bis(diphenylphosphino) ferrocenepalladium(II) dichloromethane adduct
Pd₂Cl₂(PPh₃)₂=Palladium Bis(triphenylphosphine) chloride
Pd₂(dba)₃=Tris(dibenzylidene acetone)dipalladium(0)
Pd(PPh₃)₄=tetrakis(triphenylphosphine) palladium(0)
PPh₃=Triphenylphosphine
TCDI=Thiocarbonyldiimidazole
TEA or Et₃N=Triethylamine
TFA=Trifluoroacetic Acid
TFAA=Trifluoroacetic Anhydride
THF=Tetrahydrofuran
TMSCH N₂=Trimethylsilyldiazomethane
Tris HCl or Tris-Cl=Tris[hydroxymethyl]aminomethyl hydrochloride

As used herein, unless otherwise noted, the term “anxiolytic disorders” shall be defined to include anxiety and related disorders including generalized anxiety disorder, acute stress disorder, post traumatic stress disorder, obsessive-compulsive disorder, social phobia (also known as social anxiety disorder), specific phobia, panic disorder with or without agoraphobia, agoraphobia without a history of panic disorder, anxiety disorder due to general medical condition, substance abuse induced anxiety disorder and anxiety disorder not otherwise specified (as these conditions are described by their diagnostic criteria, as listed in the Diagnostic and Statistical Manual of Mental Disorders, 4^{th} Edition, Text Revision, American Psychiatric Association, 2000, incorporated herein by reference). Anxiolytic disorders shall further include stress disorders including but not limited to hemorrhagic stress, stress-induced psychotic episodes, psychosocial dwarfism, stress headaches, stress-induced immune systems disorders such as stress-induced fever, and stress-related sleep disorders. Preferably, the anxiety or related disorder is selected from the group consisting of generalized anxiety disorder, acute stress disorder, post traumatic stress disorder and obsessive-compulsive disorder. More preferably, the anxiety and related disorder is generalized anxiety disorder.

As used herein, unless otherwise noted, the term “depression” shall be defined to include major depressive disorder (including single episode and recurrent), unipolar depression, treatment-refractory depression, resistant depression, anxious depression, dysthymia (also referred to as dysthymic disorder) as well as bipolar or manic disorders. Further, the term “depression” shall encompass any major depressive disorder, dysthymic disorder and depressive disorder not otherwise specific as defined by their diagnostic criteria, as listed in the Diagnostic and Statistical Manual of Mental Disorders, 4^{th} Edition, Text Revision, American Psychiatric Association, 2000. Preferably, the depression is major depressive disorder, unipolar depression, treatment-refractory depression, resistant depression or anxious depression. More preferably, the depression is major depressive disorder.

As used herein, unless otherwise noted, the term “neurological disorders” include CNS disorders such as tinitus, spasticity, and neuropathic pain, supranuclear palsy, AIDS related dementias, multiinfarct dementia, neurodegenerative disorders such as Alzheimer's disease, Parkinson's disease, and Huntington's disease, head trauma, spinal cord trauma, ischemic neuronal damage, amyotrophic lateral sclerosis, and disorders of pain perception such as fibromyalgia and epilepsy.

As used herein, the term “pain” shall be defined to include acute, chronic, inflammatory and neuropathic pain (preferably diabetic neuropathy). Further, the pain may be centrally mediated, peripherally mediated, caused by structural tissue injury, caused by soft tissue injury or caused by progressive disease. Any centrally mediated, peripherally mediated, structural tissue injury, soft tissue injury or progressive disease related pain may be acute or chronic.

As used herein, unless otherwise noted, pain shall include inflammatory pain, centrally mediated pain, peripherally mediated pain, visceral pain, structural related pain, cancer pain, soft tissue injury related pain, progressive disease related pain, neuropathic pain, acute pain from acute injury, acute pain from trauma, acute pain from surgery, headache, dental pain, back pain (preferably lower back pain), chronic pain from neuropathic conditions and chronic pain from post-stroke conditions.

“Nerve tissue” as used herein refers to any vertebrate nerve tissue, particularly including mammalian cells of the central nervous system (CNS) and peripheral nervous system (PNS). More particularly, nerve tissue includes spinal cord neuronal structures, peripheral nervous system nerves, and even nerve cells of the brain. “Nerve tissue injury”, “injured mammalian nerve tissue”, or “CNS or PNS nerve tissue injury” include any damage to relevant nerve tissue irrespective of cause, e.g., injuries attributable to trauma including but not limited to nerve tissue lesions, traumatically-induced compression, tumors, hemorrhage, infectious processes, spinal stenosis, or impaired blood supply.

“Treating injured mammalian nerve tissue” includes, but is not limited, to the in vivo administration of compounds, compositions, and methods of the instant invention to restore action potential or nerve impulse conduction through a nerve tissue lesion. The term may also include such administration in an effort to reduce the damaging effects of any injury to mammalian nerve tissue, whether through restoration of action potential or nerve impulse conduction, by stimulating growth or proliferation of nervous tissue, by ameliorating unwanted conditions in the extracellular microenvironment near an injury, or otherwise.

As used herein, unless otherwise noted, the term “cardiovascular diseases” shall include, for example, cardiac arrhythmia, post-myocardial infarction, and heart failure.

As used herein, unless otherwise noted, the term “sleep-wake state disorders” shall include narcolepsy; sleep apnea disorders such as central sleep apnea, obstructive sleep apnea, and mixed sleep apnea; hypersomnia, including excessive daytime sleepiness (EDS), and, in particular, hypersomnia associated with narcolepsy or sleep apnea disorder; sleep/wake disturbances associated with attention deficit hyperactive disorder (ADHD); circadian rhythm abnormalities such as delayed sleep phase syndrome, advance sleep phase syndrome, non-24 hour sleep/wake disorder, jet lag, or shift-work disorder; parasomnia disorders such as somnambulism, pavor nocturnus, REM sleep behavior disorder, sleep bruxism, or sleep enuresis; sleep-related movement disorders such as sleep bruxism, restless legs syndrome, or periodic limb movement; insomnia, including extrinsic insomnia, psychophysiologic insomnia, drug-dependent insomnia, or alcohol-dependent insomnia; sleep/wake disturbances associated with mental disorders such as depression, anxiety, schizoprenia, or other psychotic disorders; sleep/wake disturbances associated with neurological disorders such as migraine, epilepsy, Parkinson's disease, or Alzheimer's disease; and sleep/wake disturbances associated with fibromyalgia, headaches, gastroesophageal reflux disease, coronary artery ischemia, cardiac arrhythmias, abnormal swallowing, choking, or laryngospasm.

As used herein, unless otherwise noted the term “substance” when referring to substances of abuse and/or addiction shall include any legal or illegal substance to which a subject or patient may develop an addiction. Suitable examples include, but are not limited to alcohol, amphetamines (such as, for example, 3,4-methylene-dioxy-N-methylamphetamine, also known as “MDMA” or “ecstasy”), cannabis, hallucinogens (such as, for example, cocaine), inhalants, heroine, ketamine, Ecstacy, nicotine, oxycontin/oxycodone, codeine, morphine, opiods, phencyclidine, narcotics, or sedatives, or combinations thereof.

As used herein, unless otherwise noted, the term “substance abuse and addiction related disorders” shall include misuse, addiction, and/or dependence disorders related to substances of abuse. “Substance abuse and addiction related disorders” shall further include cravings, symptoms of withdrawal, and the like, associated with the misuse, addiction and/or dependency to substances of abuse.

As used herein, the term “obesity” shall be defined as a body mass index (BMI) of greater than or equal to about 25, preferably a BMI of greater than or equal to about 30. (The body mass index and other definitions are according to the “NIH Clinical Guidelines on the Identification and Evaluation, and Treatment of Overweight and Obesity in Adults” (1998)) Thus as used herein, the term “obesity” shall include both overweight and clinically obese subjects/patients.

As used herein, unless otherwise noted, the term “obesity-related disorders” shall include anorexia nervosa, wasting, AIDS-related weight loss, bulimia, cachexia, lipid disorders including hyperlipidemia and hyperuricemia, insulin resistance, noninsulin dependent diabetes mellitus (NIDDM, or Type II diabetes), insulin dependent diabetes mellitus (IDDM or Type I diabetes), diabetes-related complications including microangiopathic lesions, ocular lesions, retinopathy, neuropathy, and renal lesions, cardiovascular disease including cardiac insufficiency, coronary insufficiency, and high blood pressure, atherosclerosis, atheromatous disease, stroke, hypertension, Syndrome X, gallbladder disease, osteoarthritis, sleep apnea, forms of cancer such as uterine, breast, colorectal, kidney, and gallbladder, high cholesterol levels, complications of pregnancy, menstrual irregularities, hirsutism, muscular dystrophy, infertility, and increased surgical risk.

Recently, Kuo et al. (Kuo L E, Kitlinska J B, Tilan J U, et al., Nat Med 2007) disclosed evidence which suggest that NPY acts directly in the periphery on fat tissue and mediates stress-induced obesity and metabolic syndrome. Thus, manipulation of NPY2 receptor within fat tissue offers a new way to remodel fat and treat obesity and metabolic syndrome. Additionally, NPY2 receptor antagonism has anti-angiogenic/adipogenic effects and improves glucose tolerance. NPY2 receptor antagonist are therefore useful in the treatment of obesity, obesity related disorders, impaired oral glucose tolerance, elevated glucose levels, diabetes mellitus and related glucose related disorders.

As used herein, unless otherwise noted, the term “disorders responsive to modulation of endocrine function (more particularly, disorders responsive to modulation of the pituitary and/or hypothalamic gland)” include, but are not limited to elevated glucose level, pre-diabetes, impaired oral glucose tolerance, poor glycemic control, Type II Diabetes Mellitus, Syndrome X (also known as metabolic syndrome), gestational diabetes, insulin resistance, hyperglycemia and loss of muscle mass as a results of hyperglycemia (cachexia), infertility, inovulation, and the like. Further, the term “metabolic disorders” shall include disorders related to the metabolic system, including, but not limited to elevated glucose level, pre-diabetes, impaired oral glucose tolerance, poor glycemic control, Type II Diabetes Mellitus, Syndrome X (also known as metabolic syndrome), gestational diabetes, insulin resistance, hyperglycemia, and the like.

“Neurotrophic factor”, as used herein, refers to compounds that are capable of stimulating growth or proliferation of nervous tissue, including compounds of the instant invention and known neurotrophic factors described previously herein. Thus, the term “disorders responsive to treatment through administration of a neurotrophic factor” shall refer to any disorder which whose symptoms, pathways and/or progression may be treated and/or prevented through the use of a neurotropic factor agent.

As used herein, unless otherwise noted, the term “bone loss” refers to enhancement of bone growth or prevention of bone loss caused by conditions such as osteoporosis, osteomalacia, Paget's disease, disorders of bone homeostasis, and the like.

As used herein, unless otherwise noted, the term “infertility” shall include both male and female infertility. As used herein, unless otherwise noted, the term “inovulation” shall include inovulation regardless of underlying cause.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

One skilled in the art will recognize that, where not otherwise specified, the reaction step(s) is performed under suitable conditions, according to known methods, to provide the desired product.

One skilled in the art will recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

As used herein, unless otherwise noted, the term “leaving group” shall mean a charged or uncharged atom or group which departs during a substitution or displacement reaction. Suitable examples include, but are not limited to, Br, CI, I, mesylate, tosylate, and the like.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following:

acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following:

acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and

bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Compounds of formula (I) may be prepared according to two alternate processes, as outlined in Scheme 1 below.

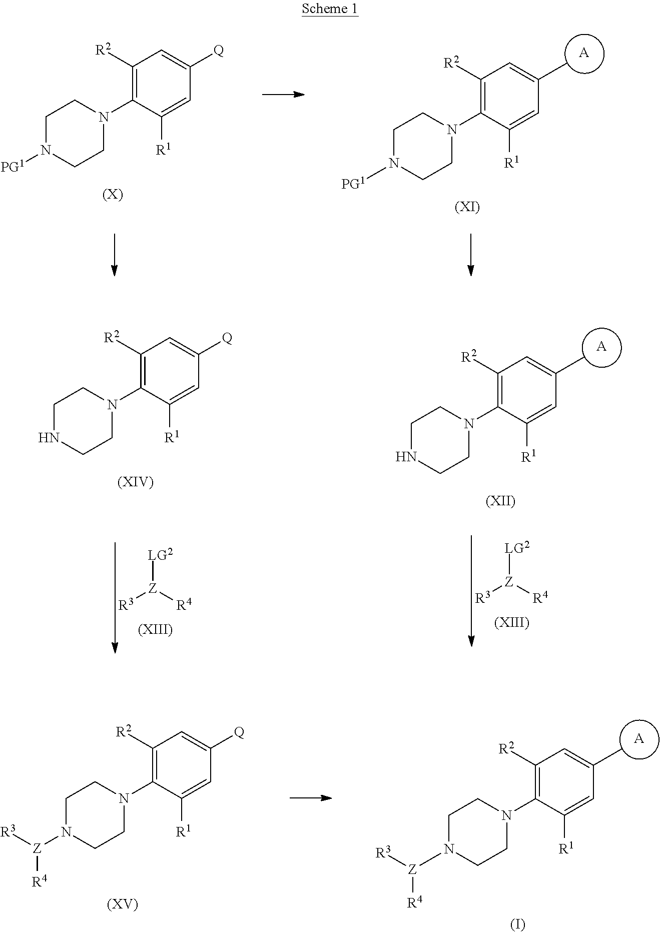

Accordingly, a suitably substituted compound of formula (X) is reacted to yield the corresponding compound of formula (XI). The compound of formula (XI) is de-protected according to known methods to yield the corresponding compound of formula (XII). The compound of formula (XII) is then reacted with a suitably substituted compound of formula (XIII), to yield the corresponding compound of formula (I).

Alternatively, a suitably substituted compound of formula (X) is de-protected according to known methods, to yield the corresponding compound of formula (XIV). The compound of formula (XIV) is reacted with a suitably substituted compound of formula (XIII-A), to yield the corresponding compound of formula (XV). The compound of formula (XV) is then reacted to yield the corresponding compound of formula (I). One skilled in the art will recognize that an unprotected version of the compound of formula (X) may alternatively used, thereby avoiding the de-protection step.

Schemes 2 through 4 below detail the processes by which the

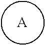

and the —ZR³R⁴ substituent groups may be attached to the piperazinyl-phenyl portion of the compound of formula (I) and unless otherwise noted, may be used in either order to yield the desired compound of formula (I).

The compound of formula (X) may be prepared according to the process outlined in Scheme 2 below.

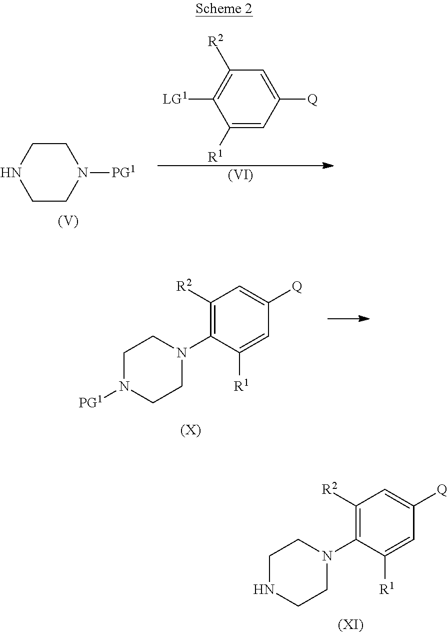

Accordingly, a suitably substituted compound of formula (V), wherein PG¹ is a suitably selected nitrogen protecting group such as BOC, CBz, benzyl, and the like, preferably BOC, a known compound or compound prepared by known methods, is reacted with a suitably substituted compound of formula (VI), wherein LG¹ is a suitably selected reactive group such as F, Cl, Br, triflate, and the like, preferably F; and wherein Q is a suitable reactive group such as Br, CI, CN, —C(O)H, —C(O)OH, —C(O)O—C₁₋₄alkyl, —C₁₋₄alkyl-C(O)OH, —C₁₋₄alkyl-NH₂, NO₂, and the like; wherein the compound of formula (VI) is preferably present in an amount in the range of from about 1.0 to about 1.5 molar equivalents; in the presence of a base such as K₂CO₃, Na₂CO₃, KOH, Hunigs' base, and the like, preferably Hunig's base; neat or in an organic solvent such as THF, DMF, NMP, acetonitrile, and the like, preferably in acetonitrile, preferably at a temperature in the range of from about 50° C. to about 80° C.; to yield the corresponding compound of formula (X).

One skilled in the art will recognize that the compound of formula (X) may be further, optionally de-protected according to known methods, to yield the corresponding compound of formula (XI). For example, wherein the compound of formula (X), PG¹ is BOC, the compound of formula (X) may be de-protected by reacting with a suitably selected acid such as HCl, TFA, and the like, in an organic solvent such as methanol, ethanol, diethyl ether, and the like.

In the synthesis of the compounds of formula (I), the —ZR³R⁴ substituent group may be attached to the piperazinyl-phenyl portion according to the process outlined in Scheme 3 below. As an example, Scheme 3 below outlines the process for attaching the —ZR³R⁴ substituent group, by reacting with a suitably substituted compound of formula (XII). One skilled in the art will recognize that as described in Scheme 1 above, the —ZR³R⁴ substituent group may alternatively, be reacted with a suitably substituted compound of formula (XIV), according to the process conditions as described below.

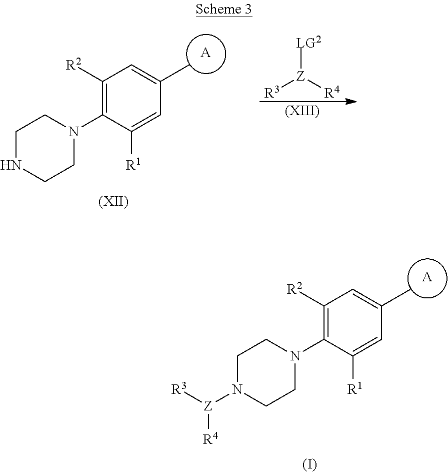

Accordingly, a suitably substituted compound of formula (XII), is reacted with a suitably substituted compound of formula (XIII), herein LG² is a suitably selected leaving group such as iodide, bromide, chloride, tosylate, mesylate, and the like, a known compound or compound prepared by known methods, wherein the compound of formula (XIII) is preferably present in an amount in the range of form about 1.0 to about 1.5 molar equivalents; in the presence of a base such as K₂CO₃, Na₂CO₃, NaH, and the like, preferably K₂CO₃; wherein the base is preferably present in an amount in the range of from about 3.0 to about 4.0 molar equivalents; in an organic solvent such as THF, DMF, and the like, preferably DMF; at a temperature between room temperature and reflux temperature, to yield the corresponding compound of formula (I).

Alternatively, the compound of formula (XII) is reacted with a suitably substituted compound of formula (XIII), wherein LG² is a carboxyl group, a known compound or compound prepared by known methods, wherein the compound of formula (XIII) is preferably present in an amount in the range of form about 1.0 to about 1.5 molar equivalents; in the presence of a suitably selected reducing agent such as NaBH(OAc)₃, NaBH₃CN, and the like; wherein the reducing agent is preferably presence in an amount in the range of from about 1.0 to about 1.5 molar equivalents; in an organic solvent such as DCM, DCE, MeOH, EtOH, and the like, preferably at about room temperature; to yield the corresponding compound of formula (I).

In the synthesis of the compounds of formula (I), the

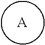

substituent group may be attached to the piperazinyl-phenyl portion according to the process outlined in Scheme 4. As an example, Scheme 4-A outlines the process for attaching the

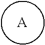

substituent group, by reacting with a suitably substituted compound of formula (X). One skilled in the art will recognize that as described in Scheme 1 above, the

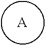

substituent group may alternatively, be reacted with a suitably substituted compound of formula (XV), according to the process conditions as described below.

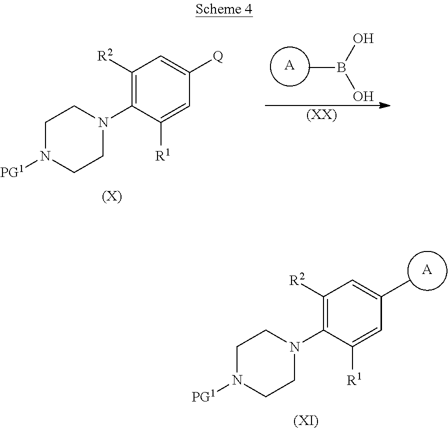

Accordingly, a suitably substituted compound of formula (X) wherein Q is Br, and the like, is reacted with a suitably substituted compound of formula (XX), a known compound or compound prepared by known methods, under Suzuki coupling conditions (for example, in the presence of a suitably selected palladium catalyst such tetrakis(triphenylphosphine) palladium (0) (Pd(PPh₃)₄), dichloro[1,1′-bis(diphenylphosphino)ferrocenepalladium(II) dichloromethane adduct (Pd(dppf)Cl₂), and the like; in the presence of a base such as sodium carbonate, potassium carbonate, and the like; in an organic solvent such as dioxane, toluene, and the like), to yield the corresponding compound of formula (XI).

Compounds of formula (XI) wherein

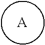

is an optionally substituted pyrazolone may alternatively be prepared by reacting a suitably substituted compound of formula (X), wherein Q is CH(O) with ethyl diazoacetate to in the presence of Lewis acid such as BF₃, in an organic solvent such as diethyl ether to form the corresponding compound, wherein the CH(O) is converted to the corresponding —C(O)—CH₂—C(O)—OCH₂CH₃ (i.e. a β-ketoester); which compound is then reacted with a suitably substituted hydrazine (a compound of the formula NH₂—NHR, wherein R is hydrogen or the desired pyrazolone substituent), in an organic solvent such as ethanol, to form the corresponding compound of formula (XI), wherein

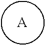

is the corresponding 1-substituted-pyrazolone via ring closure. Example 42 which follows herein, describes the preparation of a representative compound of formula (I) wherein

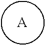

is 3-(1-isopropyl-5-oxo-4,5-dihydro-1H-pyrazolyl).

Wherein the

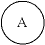

substituent group is an optionally substituted 1,2,4-triazole, the

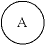

substituent may alternatively be prepared by reacting a suitably substituted compound of formula (X), wherein Q is —C(O)O—C₁₋₄alkyl, preferably —C(O)OCH₃, with hydrazine, in an organic solvent such as methanol, ethanol, and the like, to form the corresponding compound, wherein the alkyl ester is converted to the corresponding acyl hydrazid;

which compound is then reacted with a suitably substituted amidine, (a compound of the formula NH₂—C(═NH)R, wherein R is hydrogen or the desired 1,2,4-triazole substituent), in the presence of a base such as NaOCH₃, in an organic solvent such as ethanol, to form the corresponding compound of formula (XI), wherein

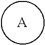

is the corresponding 5-(3-substituted-1,2,4-triazole) via ring closure. Example 60 which follows herein, describes the preparation of a representative compound of formula (I) wherein

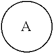

is 3-(3-methyl-1,2,4-triazolyl).

One skilled in the art will recognize that wherein the compound of formula (XI), the 1,2,4-triazole group is further substituted at the 1-position nitrogen, the above prepared compound may be further reacted with a suitably substituted alkyl halide, compound (for example, with CH₃I to incorporate a methyl substitution), in the presence of a base such as NaH, in an organic solvent such as DMF. Examples 67 and 68 which follows herein, describes the preparation of a representative compound of formula (I) wherein

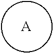

is a substituted 3-(1,5-dimethyl-[1,2,4]-triazolyl) and 3-(1-methyl-5-isopropyl-[1,2,4]-triazolyl), respectively,

Compounds of formula (XI) wherein

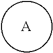

is an optionally substituted 4-(methoxycarbonyl)-oxazole may alternatively be prepared by activating a suitably substituted compound of formula (X), wherein Q is —C(O)OH by reacting with for example, serine methyl ester hydrochloride, in the presence of an organic amine base such as TEA, in an organic solvent such as THF, to form the corresponding compound, wherein the carboxylic acid is converted to the corresponding amide;

which compound is then cyclized by reacting with Burgess reagent according to known methods, to yield the corresponding 4-(methoxycarbonyl)-dihydro-oxazole substituent group; which is then dehydrated by reacting with BrCl₃C in an organic solvent such as DCM, to yield the corresponding compound of formula (XI), wherein

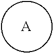

is the corresponding 4-carbomethoxy-1,2,4-oxazole. Examples 16 and 17 which follow herein, describes the preparation of a representative compounds of formula (I) wherein

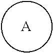

is 2-(4-(methoxycarbonyl)-4,5-dihydro-oxazole) and 2-(4-(methoxycarbonyl)-oxazole), respectively.

Compounds of formula (XI) wherein

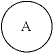

is an optionally substituted 3-(5-substituted-1,2,4-oxadiazole) may alternatively be prepared by activating a suitably substituted compound of formula (X), wherein Q is —CN by reacting with a suitably selected hydroxylamine hydrochloride, in the presence of base such as sodium carbonate, in an organic solvent such as ethanol, to form the corresponding compound, wherein the Q nitrile is converted to the corresponding hydroxy amidine;

which compound is then reacted with a suitably substituted acid chloride (a compound of the formula R—C(O)Cl, wherein R is hydrogen or the desired 1,2,4-oxadiazole substituent) to form the corresponding compound of formula (XI), wherein

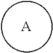

is the corresponding 3-(5-substituted-1,2,4-oxadiazole). Example 1 which follows herein, describes the preparation of a representative compound of formula (I) wherein

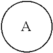

is 3-(5-ethyl-1,2,4-oxadiazole).

Compounds of formula (XI) wherein

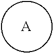

is an optionally substituted 5-(3-substituted-1,2,4-oxadiazole) may alternatively be prepared by hydrolyzing a suitably substituted compound of formula (X), wherein Q is —CN with a suitably selected base such as KOH, to convert the cyano group to the corresponding carboxylic acid; which is then converted to the corresponding acid chloride by reacting with for example, oxalyl chloride, in the presence of DMF and in an organic solvent such as DCM;

which acid chloride substituted compound is then reacted with a suitably substituted hydroxy amidine (a compound of the formula RC(═NOH)(NH₂), wherein R is hydrogen or the desired substituent), according to known methods, to form the corresponding O-acyl hydroxylamidine substituent group, which is then cyclized according to known methods, for example by reacting with sodium acetate, in an organic solvent such as t-butanol at about reflux, to form the corresponding compound of formula (XI), wherein

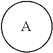

is the corresponding 5-(3-substituted-[1,2,4]-oxadiazole). Example 119 which follows herein, describes the preparation of a representative compound of formula (I) wherein

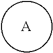

is 5-(3-methyl-[1,2,4]-oxadiazolyl).

Compounds of formula (XI) wherein

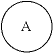

is an optionally substituted 2-(5-substituted-1,3,4-oxadiazole) may alternatively be prepared by activating a suitably substituted compound of formula (X), wherein Q is —C(O)O—C₁₋₄alkyl, such as —C(O)OCH₃, by reacting with hydrazine, according to known methods, to form the corresponding compound, wherein the Q alkyl ester substituent group is converted to the corresponding hydrazide;

which compound is acylated by reacting with a suitably substituted acid chloride (a compound of the formula R—C(O)Cl, wherein R is hydrogen or the desired substituent), and then cyclized with for example, Burgess reagent, to form the corresponding compound of formula (XI), wherein

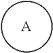

is the corresponding 2-(5-substituted-1,3,4-oxadiazole). Example 23 which follows herein, describes the preparation of a representative compound of formula (I) wherein

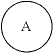

is 2-(5-methyl-1,3,4-oxadiazole).

Compounds of formula (XI) wherein

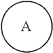

is 5-(3-methyl-[1,2,4]-thiadiazolyl) may alternatively be prepared reacting a compound of formula (X), wherein Q is cyano with a suitably selected base, according to known methods, to form the corresponding compound wherein the Q cyano group is converted to a corresponding amido group (i.e. —C(O)—NH₂);

the amido group is then converted to the corresponding thioamido (i.e. —C(S)—NH₂), by reacting with a suitably selected source of sulfur, for example Lawesson's reagent, according to known methods;

the thioamido substituted compound is then reacted with dimethylacetamide dimethylacetal, according to known methods, to yield the corresponding compound wherein the thioamido group has been reacted to yield the corresponding —C(S)—N═CH(CH₃)—N(CH₃)₂ substituted compound;

the —C(S)—N═CH(CH₃)—N(CH₃)₂ substituted compound is then cyclized, according to known methods, for example, by reacting with hydroxylamine-O-sulfonic acid in the presence of pyridine, to yield the corresponding compound of formula (XI), wherein

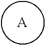

is the corresponding 5-(3-methyl-[1,2,4]-thiadiazolyl). Example 88 which follows herein, describes the preparation of a representative compound of formula (I) wherein

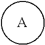

is 5-(3-methyl-[1,2,4]-thiadiazole).

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.1-1000 mg or any range therein, and may be given at a dosage of from about 0.01-300 mg/kg/day, or any range therein, preferably from about 0.5-50 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 10,000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating disorders described in the present invention may also be carried out using a pharmaceutical composition comprising any of the compounds as defined herein and a pharmaceutically acceptable carrier. The pharmaceutical composition may contain between about 0.01 mg and 1000 mg of the compound, or any range therein; preferably about 10 to 500 mg of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders mediated by the NPY Y2 receptor is required.

The daily dosage of the products may be varied over a wide range from 0.01 to 10,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 50 mg/kg of body weight per day, or any range therein. Preferably, the range is from about 0.5 to about 15.0 mg/kg of body weight per day, or any range therein. More preferably, from about 1.0 to about 10.0 mg/kg of body weight per day, or any range therein. More preferably, from about 1.0 to about 5.0 mg/kg of body weight per day, or any range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter. The examples which follow herein are only meant to suggest methods of practicing the invention. Ones skilled in the art may find other methods of practicing the invention, which are obvious to them. However, those methods are deemed to be within the scope of this invention.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like. Unless otherwise noted, the materials used in the examples were obtained from readily available commercial sources or synthesized by standard methods known to those skilled in the art.

### Purification and Analytical Methods

Mass spectra were obtained on an Agilent series 1100 MSD using electrospray ionization (ESI) in either positive or negative modes as indicated. mass corresponds to the exact mass.

Thin-layer chromatography was performed using Merck silica gel 60 F₂₅₄ 2.5 cm×7.5 cm 250 μm or 5.0 cm×10.0 cm 250 μm pre-coated silica gel plates. Preparative thin-layer chromatography was performed using EM Science silica gel 60 F₂₅₄ 20 cm×20 cm 0.5 mm pre-coated plates with a 20 cm×4 cm concentrating zone.

NMR spectra were obtained on either a Bruker model DPX400 (400 MHz) or DPX500 (500 MHz) spectrometer. The format of the ¹H NMR data below is: chemical shift in ppm down field of the tetramethylsilane reference (multiplicity, coupling constant J in Hz, integration).

Normal phase flash column chromatography (FCC) was typically performed with RediSep® silica gel columns using either 2 M ammonia in methanol/dichloromethane or hexanes/ethyl acetate as eluents.

Chiral chromatography was performed using supercritical fluid chromatography (SFC)HPLC on a Chiralpak AD-H column (Chiral Technologies), eluting with isocratic 20% TEA/MeOH/CO₂ under 100 bar pressure at 25° C. Analytical: 4.6×250 mm column, 2 mL/min flow rate. Preparative: 21×250 mm column, 37.5 mL/min flow rate.

Preparative Reversed-Phase HPLC was performed on a Gilson® instrument under the following conditions: Column: YMC-Pack ODS-A, 5 μm, 75×30 mm; Flow rate: 25 mL/min; Detection: λ=220 & 254 nm; Gradient (acetonitrile/water, 0.05% trifluoroacetic acid): 15% acetonitrile/85% water to 99% acetonitrile/1% water ramp over 20 min; or on an Agilent® 1100 Series instrument under the following conditions: Column: Phenomenex Gemini, 5 μm, 100×30 mm; Flow rate: 30 mL/min; Detection: λ=220 & 254 nm; Gradient (acetonitrile/water, 20 mM NH₄OH): 5% acetonitrile/95% water to 99% acetonitrile/1% water ramp over 20 min.

### Synthetic Methods:

Unless otherwise stated, reaction solutions were stirred at room temperature. Chemical names were generated using ChemDraw Version 6.0.2 (CambridgeSoft, Cambridge, Mass.).

Representative compounds of formula (I) of the present invention were prepared as described in the Examples which follow herein.

### Example 1

### N,N-Diethyl-2-{4-[4-(5-ethyl-[1,2,4]oxadiazol-3-yl)-2-fluoro-phenyl]-piperazin-1-yl}-2-phenyl-acetamide (Compound #3)

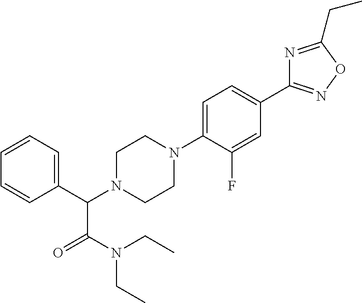

### Step A: 4-(4-Cyano-2-fluoro-phenyl)-piperazine-1-carboxylic acid tert-butyl ester

A solution of 3,4-difluoro-benzonitrile (25 g, 179.7 mmol), piperazine-1-carboxylic acid tert-butyl ester (37.5 g, 201.3 mmol) and triethylamine (35 mL) in ACN (400 mL) was refluxed for 60 h. The solvent was then removed and the resulting orange/white solid was dissolved in CH₂Cl₂ and washed with water. The aqueous washings were extracted with CH₂Cl₂ and the combined organics were dried over Na₂SO₄. The resulting residue was recrystallized from hot ethanol to yield the title compound as colorless needles.

### Step B: 4-[2-Fluoro-4-(N-hydroxycarbamimidoyl)-phenyl]-piperazine-1-carboxylic acid tert-butyl ester

To a solution of the product from Step A (2.29 g) in EtOH (70 mL) was added NH₂OH.HCl (2.60 g) followed by Na₂CO₃ (3.97 g). The resulting heterogeneous mixture was heated at 80° C. for 12 h. After cooling to room temperature, the EtOH was removed in vacuo and the residue partitioned between EtOAc and H₂O. The combined organic layers were dried over Na₂SO₄, filtered and concentrated in vacuo to yield the title compound as a white solid.

MS (electrospray): exact mass for C₁₆H₂₃FN₄O₃, 338.18; found m/z 339.5 [M+H]⁺.

### Step C: 4-[4-(5-Ethyl-[1,2,4]oxadiazol-3-yl)-2-fluoro-phenyl]-piperazine-1-carboxylic acid tert-butyl ester

To a heterogeneous mixture of the product from Step B (200 mg) in THF (4 mL) was added DIPEA (0.113 mL) followed by propionyl chloride (0.049 mL). The resulting mixture became homogeneous and after 10 min a white precipitate was observed. The resulting mixture was then heated at 155° C. in the microwave for 20 min. The resulting solution was concentrated in vacuo and chromatographed on SiO₂ (Hexanes to 25% EtOAc/Hexanes) to yield the title compound.

### Step D: N,N-Diethyl-2-{4-[4-(5-ethyl-[1,2,4]oxadiazol-3-yl)-2-fluoro-phenyl]-piperazin-1-yl}-2-phenyl-acetamide

To a solution of the product from Step C (150 mg) in CH₂Cl₂ (5 mL) was added TFA (2.5 mL). After 40 min The resulting mixture was concentrated in vacuo, the residue was dissolved in DMF (5 mL) and treated with K₂CO₃ (165 mg) followed by 2-bromo-N,N-diethyl-2-phenyl-acetamide (118 mg). The resulting mixture was heated at 50° C. overnight and then diluted with H₂O and extracted with Et₂O. The combined organic layers were dried over Na₂SO₄, filtered and concentrated in vacuo to yield a residue that was chromatographed on SiO₂ (Hexanes to 100% EtOAc/Hexanes) to yield the title compound.

MS (electrospray): exact mass for C₂₆H₃₂FN₅O₂, 465.25; found m/z 466.6 [M+H]⁺

¹H NMR (500 MHz, CDCl₃): 7.75 (dd, J=8.35, 1.73 Hz, 1H), 7.69 (dd, J=13.57, 1.89 Hz, 1H), 7.47-7.43 (m, 2H), 7.39-7.30 (m, 3H), 6.97-6.92 (m, 1H), 4.26 (s, 1H), 3.51-3.36 (m, 2H), 3.33-3.14 (m, 6H), 2.97-2.91 (q, J=7.62 Hz, 2H), 2.77-2.65 (m, 4H), 1.43 (t, J=7.62 Hz, 3H), 1.12-1.02 (m, 6H).

### Example 2

### N,N-Diethyl-2-{4-[2-fluoro-4-(5-methyl-[1,2,4]oxadiazol-3-yl)-phenyl]-piperazin-1-yl}-2-phenyl-acetamide (Compound #67)

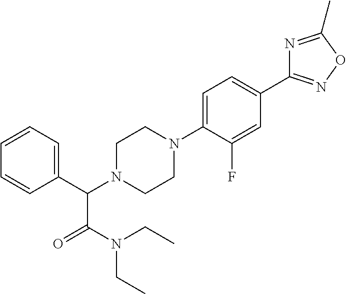

N,N-Diethyl-2-{4-[2-fluoro-4-(5-methyl-[1,2,4]oxadiazol-3-yl)-phenyl]-piperazin-1-yl}-2-phenyl-acetamide was prepared according to the procedure as described in Example 1 by reacting 200 mg of the product prepared as in Example 1, Step B and 0.040 mL of acetyl chloride to yield the title compound.

MS (electrospray): exact mass for C₂₅H₃₀FN₅O₂, 451.24; found m/z 452.5 [M+H]⁺

¹H NMR (500 MHz, CDCl₃): 7.74 (dd, J=8.41, 1.70 Hz, 1H), 7.68 (dd, J=13.58, 1.91 Hz, 1H), 7.47-7.43 (m, 2H), 7.38-7.29 (m, 3H), 6.97-6.93 (m, 1H), 4.26 (s, 1H), 3.51-3.36 (m, 2H), 3.32-3.15 (m, 6H), 2.77-2.65 (m, 4H), 2.62 (s, 3H), 1.12-1.02 (m, 6H).

### Example 3

### N,N-Diethyl-2-{4-[2-fluoro-4-(5-propyl-[1,2,4]oxadiazol-3-yl)-phenyl]-piperazin-1-yl}-2-phenyl-acetamide (Compound #68)

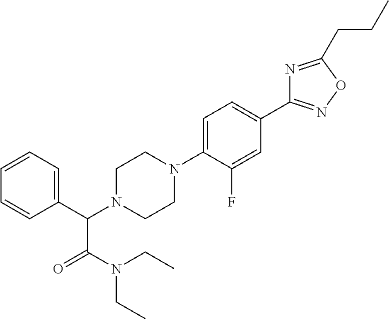

N,N-Diethyl-2-{4-[2-fluoro-4-(5-propyl-[1,2,4]oxadiazol-3-yl)-phenyl]-piperazin-1-yl}-2-phenyl-acetamide was prepared according to the procedure as described in Example 1 reacting 200 mg of the product prepared as in Example 1, Step B and 0.058 mL of butyryl chloride to yield the title compound.

MS (electrospray): exact mass for C₂₇H₃₄FN₅O₂, 479.27; found m/z 480.6 [M+H]⁺

¹H NMR (500 MHz, CDCl₃): 7.75 (dd, J=8.38, 1.78 Hz, 1H), 7.69 (dd, J=13.54, 1.90 Hz, 1H), 7.47-7.44 (m, 2H), 7.38-7.30 (m, 3H), 6.98-6.93 (m, 1H), 4.25 (s, 1H), 3.50-3.37 (m, 2H), 3.32-3.15 (m, 6H), 2.89 (t, J=7.50 Hz, 2H), 2.76-2.65 (m, 4H), 1.92-1.85 (m, 2H), 1.09 (t, J=7.09, 3H), 1.07-1.02 (m, 6H).

### Example 4

### N,N-Diethyl-2-{4-[2-fluoro-4-(5-isopropyl-[1,2,4]oxadiazol-3-yl)-phenyl]-piperazin-1-yl}-2-phenyl-acetamide (Compound #69)

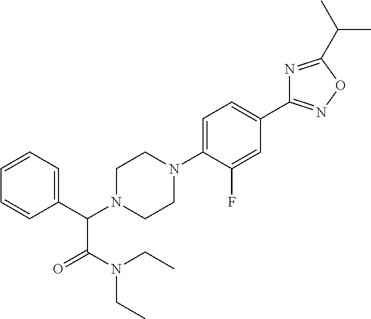

N,N-Diethyl-2-{4-[2-fluoro-4-(5-isopropyl-[1,2,4]oxadiazol-3-yl)-phenyl]-piperazin-1-yl}-2-phenyl-acetamide was prepared according to the procedure as described in Example 1 reacting 200 mg of the product prepared as in Example 1, Step B and 0.062 mL of isobutyryl chloride to yield the title compound.

MS (electrospray): exact mass for C₂₇H₃₄FN₅O₂, 479.27; found m/z 480.6 [M+H]⁺

¹H NMR (500 MHz, CDCl₃): 7.76 (dd, J=8.37, 1.73 Hz, 1H), 7.70 (dd, J=13.57, 1.91 Hz, 1H), 7.48-7.42 (m, 2H), 7.39-7.29 (m, 3H), 6.98-6.92 (m, 1H), 4.26 (s, 1H), 3.52-3.34 (m, 2H), 3.33-3.14 (m, 7H), 2.78-2.64 (m, 4H), 1.44 (d, J=6.97 Hz, 6H), 1.11-1.02 (m, 6H).

### Example 5

### N,N-Diethyl-2-(4-{4-[5-(1-ethyl-propyl)-[1,2,4]oxadiazol-3-yl]-2-fluoro-phenyl}-piperazin-1-yl)-2-phenyl-acetamide (Compound #70)

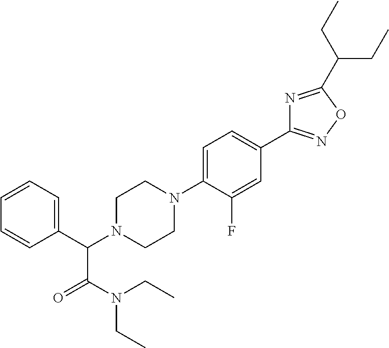

N,N-Diethyl-2-(4-{4-[5-(1-ethyl-propyl)-[1,2,4]oxadiazol-3-yl]-2-fluoro-phenyl}-piperazin-1-yl)-2-phenyl-acetamide was prepared according to the procedure as described in Example 1 reacting 200 mg of the product prepared as in Example 1, Step B and 0.077 mL of 2-ethylbutyryl chloride to yield the title compound.

MS (electrospray): exact mass for C₂₉H₃₈FN₅O₂, 507.30; found m/z 508.6 [M+H]⁺

¹H NMR (500 MHz, CDCl₃): 7.77 (dd, J=8.37, 1.72 Hz, 1H), 7.71 (dd, J=13.53, 1.89 Hz, 1H), 7.48-7.43 (m, 2H), 7.38-7.29 (m, 3H), 6.98-6.92 (m, 1H), 4.26 (s, 1H), 3.50-3.37 (m, 2H), 3.33-3.14 (m, 6H), 2.94-2.87 (m, 1H), 2.78-2.64 (m, 4H), 1.90-1.73 (m, 4H), 1.12-1.02 (m, 6H), 0.94-0.88 (m, 6H).

### Example 6

### N,N-Diethyl-2-{4-[4-(3-ethyl-[1,2,4]oxadiazol-5-yl)-2-fluoro-phenyl]-piperazin-1-yl}-2-phenyl-acetamide (Compound #71)

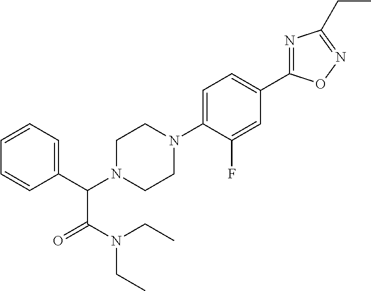

### Step A 2-chloro-N,N-diethyl-2-phenyl-acetamide

To a mixture of 2-chloro-2-phenylacetyl chloride (1.6 mL, 10 mmol) and TEA (2.8 mL, 20 mmol) in DCE (50 mL) was added diethylamine (1.1 mL. 10 mmol). The resulting mixture was stirred at room temperature for 4 h. H₂O (50 mL) was then added. The organic layer was separated and concentrated to yield the title compound.

### Step B. N,N-diethyl-2-phenyl-2-piperazin-1-yl-acetamide trifluoro-acetic acid salt

To a mixture of 2-chloro-N,N-diethyl-2-phenyl-acetamide (10 mmol) and K₂CO₃ (20 mmol) in DMF (30 mL) was added piperazine-1-carboxylic acid tert-butyl ester (1.6 g, 10 mmol). The resulting mixture was stirred at 50° C. for 3 h. To the resulting mixture was added H₂O (500 mL). After H₂O was decanted out, the semi-solid collected and re-dissolved into CF₃COOH/CH₂Cl₂ (10/50 mL). The resulting mixture was stirred at room temperature for 16 h, then concentrated to yield the title compound.

### Step C. 5-(4-bromo-3-fluoro-phenyl)-3-ethyl-[1,2,4]oxadiazole

To a mixture of 4-bromo-3-fluoro-benzoic acid (13 mmol), and DMF (0.5 mL) in CH₂Cl₂ (50 mL) was added (COCl)₂ (50 mL, 2M in CH₂Cl₂) was added. After 1 h, the resulting mixture was concentrated and pumped dry. The residue was re-dissolved into pyridine (50 mL), N-hydroxy-propionamidine (13 mmol) was added and the resulting mixture was heated 90° C. for 16 h. H₂O (300 mL) was then added to the resulting mixture. The product precipitated out and was collected via filtration to yield the title compound.

### Step D: N,N-diethyl-2-{4-[4-(3-ethyl-[1,2,4]oxadiazol-5-yl)-2-fluoro-phenyl]-piperazin-1-yl}-2-phenyl-acetamide

N,N-diethyl-2-phenyl-2-piperazin-1-yl-acetamide. trifluoro-acetic acid salt prepared as in STEP B above (1 mmol), 5-(4-bromo-3-fluoro-phenyl)-3-ethyl-[1,2,4]oxadiazole (1 mmol) prepared as in STEP C above, Cs₂CO₃ (3 mmol), Pd₂ dba₃ (0.01 mmol), and Binap (0.04 mmol), in xylene (3 ml) was heated at 100° C. for 16 h. After being cooled down, PTLC yielded the title compound.

MS (ESI): mass calcd. for C₂₆H₃₂FN₅O₂, 465.3; m/z found, 466.3 [M+H]⁺

¹H NMR (CDCl₃): 7.85-7.70 (m, 2H), 7.48-7.46 (m, 2H), 7.40-7.32 (m, 3H), 6.98-6.94 (m, 1H), 4.68 (s, 1H), 3.51-3.37 (m, 2H), 3.32-3.15 (m, 6H), 2.85-2.65 (m, 6H), 1.38 (t, J=7.6, 3H), 1.11 (t, J=7.1, 3H), 1.06 (t, J=7.1, 3H)

### Example 7

### 2-{4-[4-(5-tert-Butyl-[1,2,4]oxadiazol-3-yl)-2-fluoro-phenyl]-piperazin-1-yl}-N,N-diethyl-2-phenyl-acetamide (Compound #75)

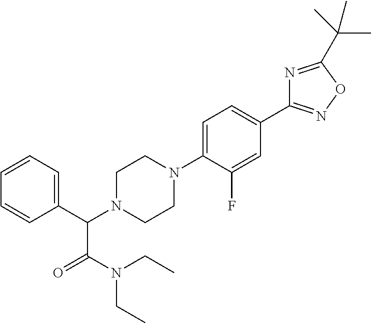

2-{4-[4-(5-tert-Butyl-[1,2,4]oxadiazol-3-yl)-2-fluoro-phenyl]-piperazin-1-yl}-N,N-diethyl-2-phenyl-acetamide was prepared according to the procedure as described in Example 1 reacting 231 mg of the product prepared as in Example 1, Step B and 0.084 mL of pivaloyl chloride to yield the title compound.

MS (electrospray): exact mass for C₂₈H₃₆FN₅O₂, 493.29; found m/z 494.6 [M+H]⁺

¹H NMR (500 MHz, CDCl₃): 7.76 (dd, J=8.39, 1.73 Hz, 1H), 7.70 (dd, J=13.54, 1.86 Hz, 1H), 7.48-7.43 (m, 2H), 7.39-7.29 (m, 3H), 6.98-6.91 (m, 1H), 4.26 (s, 1H), 3.51-3.37 (m, 2H), 3.33-3.14 (m, 6H), 2.78-2.64 (m, 4H), 1.47 (s, 9H), 1.12-1.00 (m, 6H).

### Example 8

### 2-(4-{4-[5-(2,2-Dimethyl-propyl)-[1,2,4]oxadiazol-3-yl]-2-fluoro-phenyl}-piperazin-1-yl)-N,N-diethyl-2-phenyl-acetamide (Compound #79)

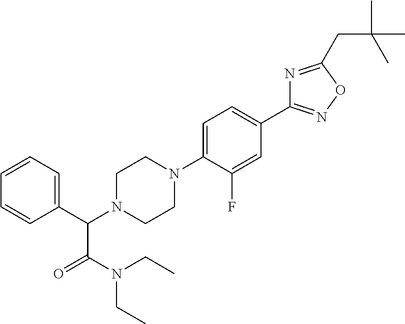

2-(4-{4-[5-(2,2-Dimethyl-propyl)-[1,2,4]oxadiazol-3-yl]-2-fluoro-phenyl}-piperazin-1-yl)-N,N-diethyl-2-phenyl-acetamide was prepared according to the procedure as described in Example 1 reacting the product prepared as in Example 1, Step B (228 mg) and 3,3-dimethylbutyryl chloride (0.089 mL) to yield the title compound.

MS (electrospray): exact mass for C₂₉H₃₈FN₅O₂, 507.30; found m/z 508.6 [M+H]⁺

¹H NMR (500 MHz, CDCl₃): 7.77 (dd, J=8.31, 1.78 Hz, 1H), 7.71 (dd, J=13.55, 1.92 Hz, 1H), 7.48-7.43 (m, 2H), 7.39-7.29 (m, 3H), 6.98-6.92 (m, 1H), 4.26 (s, 1H), 3.51-3.36 (m, 2H), 3.32-3.15 (m, 6H), 2.82 (s, 2H), 2.78-2.64 (m, 4H), 1.12-1.02 (m, 15H).

### Example 9

### N,N-Diethyl-2-{4-[2-fluoro-4-(3-isopropyl-[1,2,4]oxadiazol-5-yl)-phenyl]-piperazin-1-yl}-2-phenyl-acetamide (Compound #80)

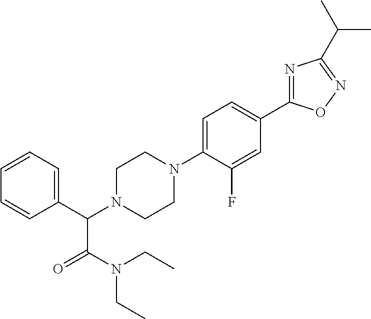

The title compound was prepared according to the process outlined in Example 6 above, with the appropriate substituent changes.

MS (ESI): mass calcd. for C₂₇H₃₄FN₅O₂, 479.3; m/z found, 480.3 [M+H]⁺

¹H NMR (CDCl₃): 7.83-7.70 (m, 2H), 7.48-7.44 (m, 2H), 7.40-7.32 (m, 3H), 6.94 (t, J=8.5, 1H), 4.68 (s, 1H), 3.51-3.37 (m, 2H), 3.32-3.15 (m, 7H), 2.87-2.65 (m, 4H), 1.38 (d, J=6.9, 6H), 1.11 (t, J=7.1, 3H), 1.06 (t, J=7.1, 3H)

### Example 10

### 2-{4-[4-(5-Cyclopropyl-[1,2,4]oxadiazol-3-yl)-2-fluoro-phenyl}-piperazin-1-yl]-N,N-diethyl-2-phenyl-acetamide (Compound #88)

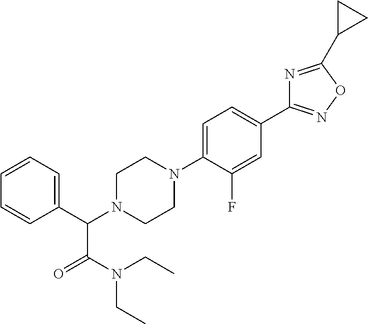

2-{4-[4-(5-Cyclopropyl-[1,2,4]oxadiazol-3-yl)-2-fluoro-phenyl]-piperazin-1-yl}-N,N-diethyl-2-phenyl-acetamide was prepared according to the procedure as described in Example 1 reacting 152 mg of the product prepared as in Example 1, Step B and 0.041 mL of cyclopropanecarbonyl chloride to yield the title compound.

MS (electrospray): exact mass for C₂₇H₃₂FN₅O₂, 477.25; found m/z 478.6 [M+H]⁺

¹H NMR (500 MHz, CDCl₃): 7.72 (dd, J=8.40, 1.85 Hz, 1H), 7.65 (dd, J=13.56, 1.89 Hz, 1H), 7.48-7.43 (m, 2H), 7.39-7.29 (m, 3H), 6.97-6.90 (m, 1H), 4.26 (s, 1H), 3.51-3.35 (m, 2H), 3.33-3.13 (m, 6H), 2.77-2.63 (m, 4H), 2.25-2.19 (m, 1H), 1.31-1.19 (m, 4H), 1.12-1.01 (m, 6H).

### Example 11

### 2-{4-[4-(5-Cyclobutyl-[1,2,4]oxadiazol-3-yl)-2-fluoro-phenyl]-piperazin-1-yl}-N,N-diethyl-2-phenyl-acetamide (Compound #89)

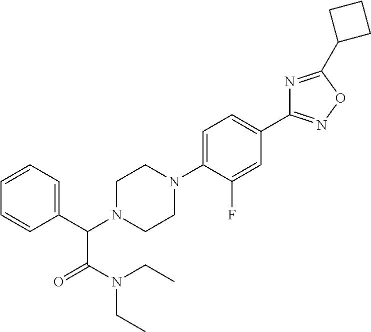

2-{4-[4-(5-Cyclobutyl-[1,2,4]oxadiazol-3-yl)-2-fluoro-phenyl]-piperazin-1-yl}-N,N-diethyl-2-phenyl-acetamide was prepared according to the procedure as described in Example 1 reacting 152 mg of the product prepared as in Example 1, Step B and 0.051 mL of cyclobutanecarbonyl chloride to yield the title compound.

MS (electrospray): exact mass for C₂₈H₃₄FN₅O₂, 491.27; found m/z 492.6 [M+H]⁺

¹H NMR (500 MHz, CDCl₃): 7.76 (dd, J=8.34, 1.74 Hz, 1H), 7.70 (dd, J=13.55, 1.89 Hz, 1H), 7.48-7.42 (m, 2H), 7.39-7.29 (m, 3H), 6.98-6.91 (m, 1H), 4.26 (s, 1H), 3.83-3.74 (m, 1H), 3.51-3.36 (m, 2H), 3.33-3.14 (m, 6H), 2.78-2.63 (m, 4H), 2.57-2.42 (m, 4H), 2.20-2.03 (m, 2H), 1.12-1.00 (m, 6H).

### Example 12

### 2-{4-[4-(5-sec-Butyl-[1,2,4]oxadiazol-3-yl)-2-fluoro-phenyl]-piperazin-1-yl}-N,N-diethyl-2-phenyl-acetamide (Compound #97)

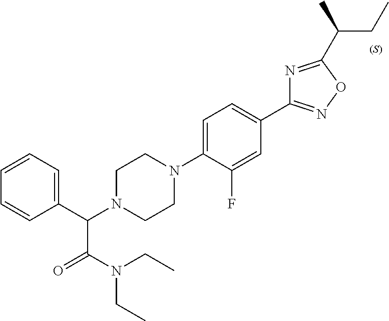

### Step A: 4-[4-(5-sec-Butyl-[1,2,4]oxadiazol-3-yl)-2-fluoro-phenyl]-piperazine-1-carboxylic acid tert-butyl ester

To a solution of (S)-2-methylbutyric acid (0.034 mL) in THF (2 mL) was added Cl₃CCN (0.047 mL) followed by resin bound PPh₃ (310 mg) and the resulting mixture was heated at 105° C. for 7 min in the microwave. After cooling, the product prepared as in Example 1, Step B (115 mg) and DIPEA (0.108 mL) were added and the resulting mixture heated at 155° C. for 20 min in the microwave. The resulting mixture was filtered and the resin washed with THF. The filtrate was concentrated in vacuo and chromatography on SiO₂ (Hexanes to 20% EtOAc/Hexanes) yield the title compound.

MS (electrospray): exact mass for C₂₁H₂₉FN₄O₃, 404.22; found m/z 349.5 [M-^{t}Bu]⁺, 427.6 [M+Na]⁺.

### Step B: 2-{4-[4-(5-sec-Butyl-[1,2,4]oxadiazol-3-yl)-2-fluoro-phenyl]-piperazin-1-yl}-N,N-diethyl-2-phenyl-acetamide

The title compound was prepared according to the procedure as described in Example 1, Step D reacting 27 mg of the product prepared as in Step A above to yield the title compound.

MS (electrospray): exact mass for C₂₈H₃₆FN₅O₂, 493.29; found m/z 494.6 [M+H]⁺

¹H NMR (500 MHz, CDCl₃): 7.77 (dd, J=8.41, 1.74 Hz, 1H), 7.71 (dd, J=13.42, 1.85 Hz, 1H), 7.52-7.45 (m, 2H), 7.44-7.35 (m, 3H), 6.99-6.91 (m, 1H), 3.50-3.39 (m, 2H), 3.39-3.23 (m, 6H), 3.22-3.03 (m, 4H), 3.01-2.78 (m, 2H), 1.95-1.85 (m, 1H), 1.79-1.70 (m, 1H), 1.41 (d, J=7.04 Hz, 3H), 1.11 (t, J=7.07 Hz, 3H), 1.03-0.92 (m, 6H).

### Example 13

### N,N-Diethyl-2-{4-[2-fluoro-4-(5-fluoromethyl-[1,2,4]oxadiazol-3-yl)-phenyl]-piperazin-1-yl}-2-phenyl-acetamide (Compound #105)

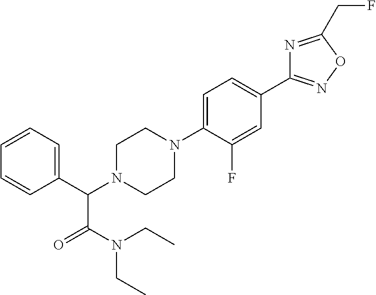

N,N-Diethyl-2-{4-[2-fluoro-4-(5-fluoromethyl-[1,2,4]oxadiazol-3-yl)-phenyl]-piperazin-1-yl}-2-phenyl-acetamide was prepared according to the procedure as described in Example 1 reacting the product prepared as in Example 1, Step B (80 mg) and 2-fluoroacetyl chloride (0.018 mL) to yield the title compound.

MS (electrospray): exact mass for C₂₅H₂₉F₂N₅O₂, 469.23; found m/z 470.6 [M+H]⁺

¹H NMR (500 MHz, CDCl₃): 7.77 (dd, J=8.36, 1.91 Hz, 1H), 7.71 (dd, J=13.50, 1.93 Hz, 1H), 7.48-7.43 (m, 2H), 7.39-7.29 (m, 3H), 6.99-6.92 (m, 1H), 5.59 (d, J=46.32 Hz, 2H), 4.27 (s, 1H), 3.52-3.36 (m, 2H), 3.32-3.14 (m, 6H), 2.78-2.65 (m, 4H), 1.12-1.01 (m, 6H).

### Example 14

### N,N-Diethyl-2-{4-[2-fluoro-4-(5-trifluoromethyl-[1,2,4]oxadiazol-3-yl)-phenyl]-piperazin-1-yl}-2-phenyl-acetamide (Compound #118)

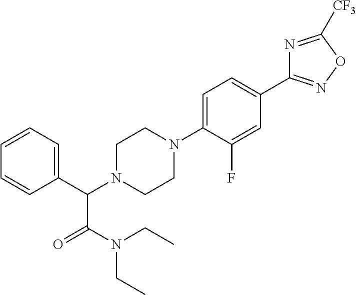

N,N-Diethyl-2-{4-[2-fluoro-4-(5-trifluoromethyl-[1,2,4]oxadiazol-3-yl)-phenyl]-piperazin-1-yl}-2-phenyl-acetamide was prepared according to the procedure as described in Example 1 reacting 185 mg of the product prepared as in Example 1, Step B and 0.076 mL of TFAA to yield the title compound.

MS (electrospray): exact mass for C₂₅H₂₇F₄N₅O₂, 505.21; found m/z 506.6 [M+H]⁺

¹H NMR (500 MHz, CDCl₃): 7.79 (dd, J=8.44, 1.90 Hz, 1H), 7.72 (dd, J=13.43, 1.95 Hz, 1H), 7.48-7.43 (m, 2H), 7.40-7.30 (m, 3H), 7.00-6.94 (m, 1H), 4.27 (s, 1H), 3.53-3.35 (m, 2H), 3.33-3.14 (m, 6H), 2.79-2.65 (m, 4H), 1.12-1.02 (m, 6H).

### Example 15

### N,N-Diethyl-2-{4-[2-fluoro-4-(3-methyl-[1,2,4]oxadiazol-5-yl)-phenyl]-piperazin-1-yl}-2-phenyl-acetamide (compound #119)

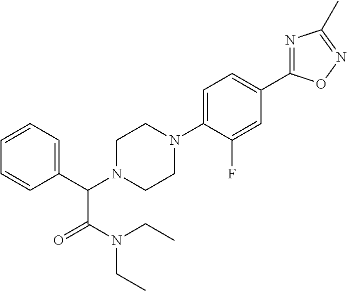

### Step A: 4-(4-Carboxy-2-fluoro-phenyl)-piperazine-1-carboxylic acid tert-butyl ester

To a heterogeneous mixture of the product prepared as in Example 1, Step A (5.07 g) in EtOH (83 mL) was added 2 N KOH (83 mL). The resulting mixture was heated at 100° C. for 14 h and the EtOH was then removed in vacuo. The aqueous layer was extracted with Et₂O and then acidified to pH 2 with 4 N HCl and extracted with CH₂Cl₂. The combined organic layers were dried over Na₂SO₄, filtered and concentrated in vacuo to yield the title compound as a white solid.

MS (electrospray): exact mass for C₁₆H₂₁FN₂O₄, 324.15; found m/z 225.4 [M-Boc]⁺, 269.4 [M-^{t}Bu]⁺, 347.5 [M+Na]⁺.

### Step B: 1,1-Dimethylethyl 4-{4-[({[(1Z)-1-aminoethylidene]amino}oxy)carbonyl]-2-fluorophenyl}piperazine-1-carboxylate

To a solution of the product of Step B (282 mg) in CH₂Cl₂ (10 mL) at 0° C. was added (COCl)₂ (0.7 mL, 2 M solution in CH₂Cl₂) dropwise. The resulting yellow mixture was treated with two drops of DMF followed by Et₃N (0.242 mL). After 20 min The resulting mixture was concentrated in vacuo to a dark orange viscous oil. The residue was re-dissolved in CH₂Cl₂ (10 mL) and treated with N-hydroxy-acetamidine (77 mg) followed by Et₃N (0.121 mL). After 14 h at room temperature. The resulting mixture was concentrated in vacuo and chromatography on SiO₂ (Hexanes to 100% EtOAc/Hexanes) to yield the title compound as a white foam.

MS (electrospray): exact mass for C₁₈H₂₅FN₄O₄, 380.19; found m/z 381.5 [M+H]⁺.

### Step C: 4-[2-Fluoro-4-(3-methyl-[1,2,4]oxadiazol-5-yl)-phenyl]-piperazine-1-carboxylic acid tert-butyl ester

To a solution of the product of Step B above (260 mg) in THF (3 mL) was added Et₃N.HCl (94 mg). The resulting mixture was heated in the microwave at 155° C. for 20 min. The resulting mixture was then concentrated in vacuo and chromatography on SiO₂ (Hexanes to 40% EtOAc/Hexanes) to yield the title compound.

MS (electrospray): exact mass for C₁₈H₂₃FN₄O₃, 362.18; found m/z 263.5 [M-Boc]⁺, 307.5 [M-^{t}Bu]⁺, 385.5 [M+Na]⁺.

### Step D: 1-[2-Fluoro-4-(3-methyl-[1,2,4]oxadiazol-5-yl)-phenyl]-piperazine

To a solution of the product of Step C above (176 mg) in CH₂Cl₂ (5 mL) was added TFA (2 mL). The resulting mixture was stirred for 3 h and then concentrated in vacuo. The residue was re-dissolved in CH₂Cl₂ and treated with Dowex 550A resin. After stirring for 1 h, the resin was removed by filtration and the filtrate was concentrated in vacuo. Chromatography on SiO₂ (CH₂Cl₂ to 8% 2 M NH₃ in MeOH/CH₂Cl₂) yielded the title compound as a white solid.

MS (electrospray): exact mass for C₁₃H₁₅FN₄O, 262.12; found m/z 263.5 [M+H]⁺.

### Step E: N,N-Diethyl-2-{4-[2-fluoro-4-(3-methyl-[1,2,4]oxadiazol-5-yl)-phenyl]-piperazin-1-yl}-2-phenyl-acetamide

To a solution of the product of Step D above (47 mg) in DMF (3 mL) was added K₂CO₃ (74 mg) followed by 2-bromo-N,N-diethyl-2-phenyl-acetamide (120 mg). The resulting mixture was heated at 50° C. overnight and then diluted with H₂O and extracted with Et₂O. The combined organic layers were dried over Na₂SO₄, filtered and concentrated in vacuo to a residue that was chromatographed on SiO₂ (Hexanes to 100% EtOAc/Hexanes) to yield the title compound.

MS (electrospray): exact mass for C₂₅H₃₀FN₅O₂, 451.24; found m/z 452.6 [M+H]⁺

¹H NMR (600 MHz, CDCl₃): 7.78 (dd, J=8.46, 1.87 Hz, 1H), 7.71 (dd, J=13.42, 1.88 Hz, 1H), 7.47-7.42 (m, 2H), 7.40-7.30 (m, 3H), 6.98-6.92 (m, 1H), 4.27 (s, 1H), 3.53-3.44 (m, 1H), 3.44-3.35 (m, 1H), 3.32-3.23 (m, 5H), 3.23-3.13 (m, 1H), 2.78-2.65 (m, 4H), 2.44 (s, 3H), 1.12-1.02 (m, 6H).

### Example 16

### 2-{4-[4-(Diethylcarbamoyl-phenyl-methyl)-piperazin-1-yl]-3-fluorophenyl}-4,5-dihydro-oxazole-4-carboxylic acid methyl ester (Compound #124)

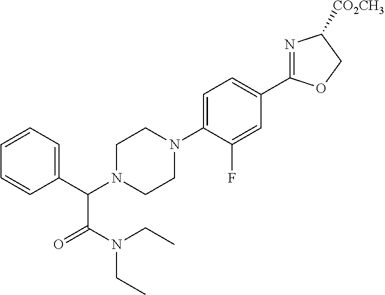

### Step A. 4-[2-Fluoro-4-(4-methoxycarbonyl-4,5-dihydro-oxazol-2-yl)-phenyl]-piperazine-1-carboxylic acid tert-butyl ester

To a solution of 4-(4-carboxy-2-fluoro-phenyl)-piperazine-1-carboxylic acid tert-butyl ester (1.01 g, 3.11 mmol) in CH₂Cl₂ (30 mL) cooled to 0° C. was added N-methylmorpholine (1 mL, 7.11 mmol) and isobutyl chloroformate (0.5 mL, 3.82 mmol). The resulting mixture was stirred at 0° C. for 1 h, then L-serine methyl ester (0.54 g, 3.47 mmol) was added in a single portion. The ice bath was removed and the resulting mixture was stirred at room temperature for 16 h. Water (50 mL) was then added, the layers separated and the aqueous layer extracted with CH₂Cl₂. The residue was purified on silica gel (120 g, Hexanes/EtOAc 20-70%) to yield a white foam.

MS (ESI): mass calcd. for C₂₀H₂₈FN₃O₆, 425.46; m/z found, 426.5 [M+H]⁺.

The white foam was dissolved in THF, Burgess reagent added (0.57 g) and the flask containing the resulting mixture placed into a preheated oil bath and refluxed for 16 h. The solvent removed and the residue purified on silica gel (40 g, Hexanes/EtOAc 0-50%) to yield the title compound as a yellow solid.

MS (ESI): mass calcd. for C₂₀H₂₆FN₃O₅, 407.44; m/z found, 408.5 [M+H]⁺

¹H NMR (CDCl₃): 7.70-7.63 (m, 2H), 6.92-6.87 (m, 1H), 4.95-4.90 (m, 1H), 4.69-4.65 (m, 1H), 4.60-4.55 (m, 1H), 3.82 (s, 3H), 3.62-3.56 (m, 4H), 3.14-3.09 (m, 4H), 1.48 (s, 9H)

### Step B. 2-{4-[4-(Diethylcarbamoyl-phenyl-methyl)-piperazin-1-yl]-3-fluoro-phenyl}-4,5-dihydro-oxazole-4-carboxylic acid methyl ester

To a CH₂Cl₂ (5 mL) solution of 4-[2-fluoro-4-(4-methoxycarbonyl-4,5-dihydro-oxazol-2-yl)-phenyl]-piperazine-1-carboxylic acid tert-butyl ester (245.1 mg, 0.60 mmol) prepared as in STEP A above was added TFA (1 mL). The resulting mixture was stirred at ambient temperature for 6 h then the solvent was removed. The resulting residue was dissolved in DMF (3 mL) and K₂CO₃ (0.23 g) added followed by 2-bromo-N,N-diethyl-2-phenyl-acetamide (0.31 g). The resulting mixture was stirred at room temperature for 16 h then diluted with water (30 mL). The resulting mixture was extracted with diethyl ether and the solvent removed from the combined organic extracts. The residue was purified on silica gel (40 g, Hexanes/EtOAc 0-75%) to yield the title compound.

MS (ESI): mass calcd. for C₂₇H₃₃FN₄O₄, 496.58; m/z found, 497.6 [M+H]⁺

¹H NMR (CDCl₃): 7.67-7.63 (m, 1H), 7.62-7.58 (m, 1H), 7.46-7.42 (m, 2H), 7.38-7.29 (m, 3H), 6.89-6.84 (m, 1H), 4.93-4.89 (m, 1H), 4.67-4.62 (m, 1H), 4.58-4.52 (m, 1H), 4.25 (s, 3H), 3.50-3.35 (m, 2H), 3.33-3.13 (m, 6H), 2.76-2.62 (m, 4H), 1.09 (t, J=7.1 Hz, 3H), 1.04 (t, J=7.1 Hz, 3H)

### Example 17

### 2-{4-[4-(Diethylcarbamoyl-phenyl-methyl)-piperazin-1-yl]-3-fluorophenyl}-oxazole-4-carboxylic acid methyl ester (Compound #129)

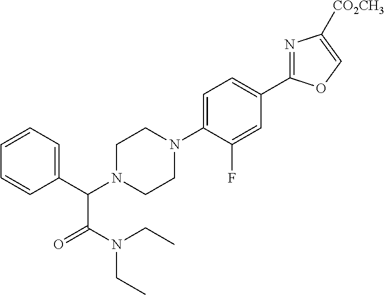

To a CH₂Cl₂ (3 mL) solution of 2-{4-[4-(diethylcarbamoyl-phenyl-methyl)-piperazin-1-yl]-3-fluoro-phenyl}-4,5-dihydro-oxazole-4-carboxylic acid methyl ester prepared as in Example 16 above was added BrCCl₃ (0.1 mL) and DBU (0.1 mL) and the resulting mixture stirred at room temperature for 3 h. The solvent was removed and the residue purified on silica gel (40 g, Hexanes/EtOAc 50-100%) to yield the title compound.

MS (ESI): mass calcd. for C27H31FN4O4, 494.57; m/z found, 495.3 [M+H]⁺

¹H NMR (CDCl₃): 8.23 (s, 1H), 7.80-7.70 (m, 2H), 7.48-7.42 (m, 2H), 7.40-7.29 (m, 3H), 6.96-6.90 (m, 1H), 4.26 (s, 1H), 3.94 (s, 3H), 3.52-3.34 (m, 2H), 3.33-3.12 (m 6H), 2.79-2.63 (m, 4H), 1.13-1.01 (m, 6H)

### Example 18

### 3-{4-[4-(Diethylcarbamoyl-phenyl-methyl)-piperazin-1-yl]-3-fluoro-phenyl}-[1,2,4]oxadiazole-5-carboxylic acid ethyl ester (Compound #130)

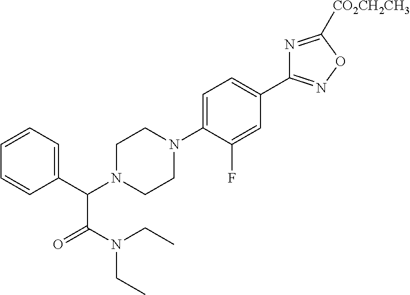

### Step A: 4-[4-(5-Ethoxycarbonyl-[1,2,4]oxadiazol-3-yl)-2-fluoro-phenyl]-piperazine-1-carboxylic acid tert-butyl ester

To a heterogeneous mixture of the product prepared as in Example 1, Step B (751 mg) in CH₂Cl₂ (20 mL) was added DIPEA (0.58 mL) followed by ethyl chlorooxoacetate (0.272 mL) dropwise. At the end of the acid chloride addition the resulting mixture was orange and homogeneous, and was heated at 40° C. for 24 h. The resulting mixture was then concentrated in vacuo to 1.71 g of orange oil and chromatographed on SiO₂ (Hexanes to 25% EtOAc/Hexanes) to yield the title compound as a white solid.

MS (electrospray): exact mass for C₂₀H₂₅FN₄O₅, 420.18; found m/z 321.1 [M-Boc]⁺, 365.1 [M-^{t}Bu]⁺, 421.2 [M+H]⁺, 443.2 [M+Na]⁺.

### Step B: 3-{4-[4-(Diethylcarbamoyl-phenyl-methyl)-piperazin-1-yl]-3-fluoro-phenyl}-[1,2,4]oxadiazole-5-carboxylic acid ethyl ester

3-{4-[4-(Diethylcarbamoyl-phenyl-methyl)-piperazin-1-yl]-3-fluoro-phenyl}-[1,2,4]oxadiazole-5-carboxylic acid ethyl ester was prepared according to the procedure as described in Example 1, Step D reacting 768 mg of the product prepared as in Step A above and to yield the title compound as a pale yellow solid.

MS (electrospray): exact mass for C₂₇H₃₂FN₅O₄, 509.24; found m/z 510.3 [M+H]⁺

¹H NMR (500 MHz, CDCl₃): 7.83 (dd, J=8.43, 1.70 Hz, 1H), 7.77 (dd, J=13.46, 1.62 Hz, 1H), 7.47-7.43 (m, 2H), 7.39-7.30 (m, 3H), 6.98-6.93 (m, 1H), 4.56 (q, J=7.13 Hz, 2H), 4.26 (s, 1H), 3.52-3.33 (m, 2H), 3.33-3.15 (m, 6H), 2.79-2.62 (m, 4H), 1.48 (t, J=7.13 Hz, 3H), 1.12-1.02 (m, 6H).

### Example 19

### N,N-Diethyl-2-{4-[4-(5-ethyl-oxazol-2-yl)-2-fluoro-phenyl]-piperazin-1-yl}-2-phenyl-acetamide (Compound #133)

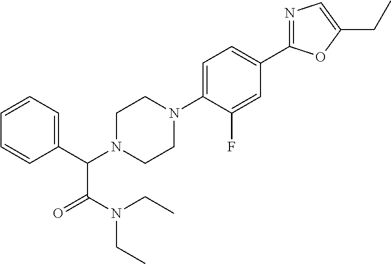

### Step A. N,N-diethyl-2-phenyl-2-piperazin-1-yl-acetamide trifluoro-acetic acid salt

To a mixture of 2-chloro-N,N-diethyl-2-phenyl-acetamide (10 mmol), as prepared in Example 6 step A, and K₂CO₃ (20 mmol) in DMF (30 mL) was added piperazine-1-carboxylic acid tert-butyl ester (1.6 g, 10 mmol). The resulting mixture was stirred at 50° C. for 3 h. To the resulting mixture was added H₂O (500 mL). After H₂O was decanted out, the semi-solid collected and re-dissolved into CF₃COOH/CH₂Cl₂ (10/50 mL). The resulting mixture was stirred at room temperature for 16 h, then concentrated to yield the title compound.

### Step B. 2-(4-bromo-3-fluoro-phenyl)-5-ethyl-oxazole

To a mixture of 4-bromo-3-fluoro-benzoic acid (25 mmol), and DMF (1 mL) in CH₂Cl₂ (100 mL) was added (COCl)₂ (100 mL, 2M in CH₂Cl₂). After 1 h, the resulting mixture was concentrated and pumped dry. The residue was re-dissolved into MeOH (100 mL) and then NH₄OH (100 mL, 5N in H₂O) was added. The resulting mixture was stirred at room temperature for 16 h. H₂O (300 mL) was then added. A solid (4-bromo-3-fluoro-benzamide) was observed to precipitated out, was collected via filtration. A mixture of 4-bromo-3-fluoro-benzamide (1.1 g, 5.0 mmol), and 1-bromo-butan-2-one (3.3 g, 20 mmol) in xylene (5 mL) was heated at 150° C. via microwave for 1 h. The title compound was isolated via PTLC and used for next step without further purification.

### Step C: N,N-diethyl-2-{4-[4-(5-ethyl-oxazol-2-yl)-2-fluoro-phenyl]-piperazin-1-yl}-2-phenyl-acetamide

A mixture of N,N-diethyl-2-phenyl-2-piperazin-1-yl-acetamide. trifluoro-acetic acid salt prepared as in STEP B above (120 mg), 2-(4-bromo-3-fluoro-phenyl)-5-ethyl-oxazole prepared as in STEP C above (135 mg, 0.50 mmol), NaOtBu (0.75 mmol), Pd₂ dba₃ (0.015 mmol), and BINAP (0.06 mmol), in xylene (3 ml) was heated at 150° C. via microwave for 1 h. After being cooled down, PTLC yielded the title compound.

MS (ESI): mass calcd. for C₂₇H₃₃FN₄O₂, 464.3; m/z found, 465.3 [M+H]⁺

¹H NMR (CDCl₃): 7.72-7.62 (m, 2H), 7.47-7.43 (m, 2H), 7.40-7.31 (m, 3H), 6.96-6.91 (m, 1H), 4.25 (s, 1H), 3.50-3.35 (m, 2H), 3.34-3.10 (m, 6H), 2.76-2.65 (m, 2H), 2.60 (m, 2H), 1.26 (t, J=7.5, 3H), 1.12-1.02 (m, 6H),

### Example 20

### 3-{4-[4-(Diethylcarbamoyl-phenyl-methyl)-piperazin-1-yl]-3-fluorophenyl}-[1,2,4]oxadiazole-5-carboxylic acid amide (Compound #144)

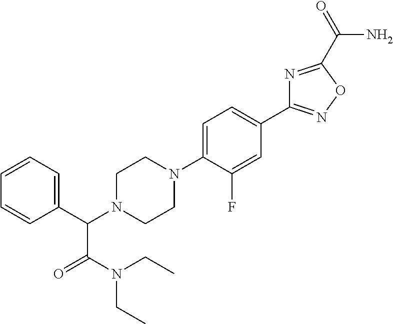

### Step A: 3-{4-[4-(Diethylcarbamoyl-phenyl-methyl)-piperazin-1-yl]-3-fluoro-phenyl}-[1,2,4]oxadiazole-5-carboxylic acid amide

To a heterogeneous mixture of the product prepared as in Example 18 (668 mg) in THF (11 mL) was added 25% aqueous NH₄OH (0.8 mL) and the homogeneous mixture was stirred for 48 h at room temperature. The resulting mixture was concentrated in vacuo and chromatography on SiO₂ (5% EtOAc/Hexanes to 100% EtOAc/Hexanes) to yield the title compound as a white solid.

MS (electrospray): exact mass for C₂₅H₂₉FN₆O₃, 480.23; found m/z 481.3 [M+H]⁺

¹H NMR (500 MHz, CDCl₃): (dd, J=8.50, 1.04 Hz, 1H), 7.71 (d, J=13.41 Hz, 1H), 7.48-7.43 (m, 2H), 7.40-7.31 (m, 3H), 7.12 (s, 1H), 6.98-6.92 (m, 1H), 6.62 (s, 1H), 4.26 (s, 1H), 3.52-3.37 (m, 2H), 3.32-3.15 (m, 6H), 2.77-2.66 (m, 4H), 1.12-1.02 (m, 6H).

### Example 21

### 2-{4-[4-(5-Cyano-[1,2,4]oxadiazol-3-yl)-2-fluoro-phenyl]-piperazin-1-yl}-N,N-diethyl-2-phenyl-acetamide (Compound #147)

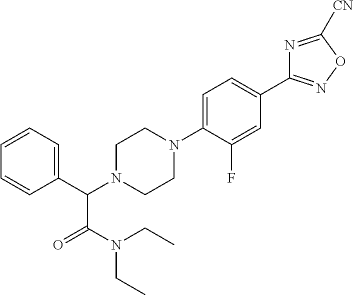

### Step A: 2-{4-[4-(5-Cyano-[1,2,4]oxadiazol-3-yl)-2-fluoro-phenyl]-piperazin-1-yl}-N,N-diethyl-2-phenyl-acetamide

To a solution of the product prepared as in Example 20 (510 mg) in THF (18 mL) was added pyridine (0.216 mL) followed by TFAA (0.206 mL) dropwise at 0° C. The resulting mixture was allowed to warm to room temperature overnight and then partitioned between H₂O and Et₂O. The combined organic layers were washed with brine, dried over Na₂SO₄, filtered and concentrated in vacuo to yield a pale orange oil. The oil was chromatographed on SiO₂ (10% Hexanes/EtOAc to 100% Hexanes/EtOAc) to yield the title compound as a pale yellow solid.

MS (electrospray): exact mass for C₂₅H₂₇FN₆O₂, 462.22; found m/z 463.3 [M+H]⁺

¹H NMR (600 MHz, CDCl₃): 7.77 (dd, J=8.44, 1.51 Hz, 1H), 7.69 (dd, J=13.37, 1.24 Hz, 1H), 7.47-7.42 (m, 2H), 7.40-7.29 (m, 3H), 7.00-6.94 (m, 1H), 4.27 (s, 1H), 3.52-3.44 (m, 1H), 3.44-3.34 (m, 1H), 3.32-3.13 (m, 6H), 2.79-2.65 (m, 4H), 1.13-1.01 (m, 6H).

### Example 22

### N,N-Diethyl-2-(4-{2-fluoro-4-[5-(N-hydroxycarbamimidoyl)-[1,2,4]oxadiazol-3-yl]-phenyl}-piperazin-1-yl)-2-phenyl-acetamide (Compound #149)

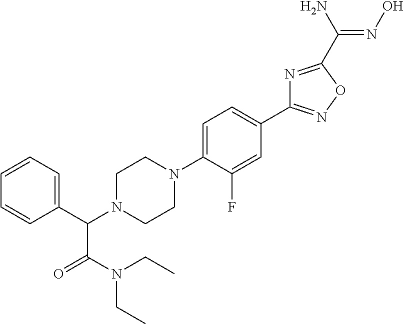

### Step A: N,N-Diethyl-2-(4-{2-fluoro-4-[5-(N-hydroxycarbamimidoyl)-[1,2,4]oxadiazol-3-yl]-phenyl}-piperazin-1-yl)-2-phenyl-acetamide

To a heterogeneous mixture of the product prepared as in Example 21 (150 mg) in EtOH (10 mL) was added NH₂OH.HCl (45 mg) followed by Na₂CO₃ (69 mg). The resulting mixture quickly became homogenous and after 30 min was concentrated in vacuo. The residue was partitioned between CH₂Cl₂ and H₂O. The combined organic layers were dried over Na₂SO₄, filtered and concentrated in vacuo to yield the title compound as a pale yellow solid.

MS (electrospray): exact mass for C₂₅H₃₀FN₇O₃, 495.24; found m/z 496.3 [M+H]⁺

¹H NMR (500 MHz, CDCl₃): 9.15-9.03 (m, 1H), 7.76-7.65 (m, 2H), 7.51-7.45 (m, 2H), 7.40-7.29 (m, 3H), 6.99-6.92 (m, 1H), 5.27 (s, 2H), 4.25 (s, 1H), 3.53-3.37 (m, 2H), 3.35-3.12 (m, 6H), 2.77-2.66 (m, 4H), 1.12-1.03 (m, 6H).

### Example 23

### N,N-Diethyl-2-{4-[2-fluoro-4-(5-methyl-[1,3,4]oxadiazol-2-yl)-phenyl]-piperazin-1-yl}-2-phenyl-acetamide (Compound #150)

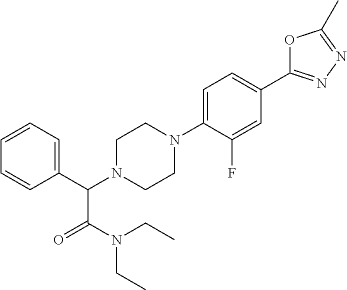

### Step A: 4-(2-Fluoro-4-methoxycarbonyl-phenyl)-piperazine-1-carboxylic acid tert-butyl ester

To a solution of the product prepared as in Example 15, Step B (2.01 g) in 3:1 benzene/MeOH (60 mL) was added TMSCHN₂ (2 M in Hexanes) until the yellow color persisted. The resulting mixture was quenched with AcOH until the solution was colorless. The resulting mixture was then concentration in vacuo to yield the title compound as a white solid.

MS (electrospray): exact mass for C₁₇H₂₃FN₂O₄, 338.16; found m/z 239.4 [M-Boc]⁺, 283.4 [M-^{t}Bu]⁺, 361.5 [M+Na]⁺.

### Step B: 4-[4-(Diethylcarbamoyl-phenyl-methyl)-piperazin-1-yl]-3-fluoro-benzoic acid methyl ester

4-[4-(Diethylcarbamoyl-phenyl-methyl)-piperazin-1-yl]-3-fluoro-benzoic acid methyl ester was prepared according to the procedure as described in Example 1, Step D reacting 2.07 g of the product prepared as in Step A above to yield the title compound as a white solid.

MS (electrospray): exact mass for C₂₄H₃₀FN₃O₃, 427.23; found m/z 428.3 [M+H]⁺.

### Step C: N,N-Diethyl-2-[4-(2-fluoro-4-hydrazinocarbonyl-phenyl)-piperazin-1-yl]-2-phenyl-acetamide

To a heterogeneous mixture of 1.53 g of the product of Step B above in MeOH (25 mL) was added NH₂NH₂.H₂O (1.73 mL) and the mixture was heated at reflux for 36 h and then concentrated in vacuo to afford 1.55 g of the title compound as a colorless foam.

MS (electrospray): exact mass for C₂₃H₃₀FN₅O₂, 427.24; found m/z 428.3 [M+H]⁺.

### Step D: N,N-Diethyl-2-{4-[2-fluoro-4-(5-methyl-[1,3,4]oxadiazol-2-yl)-phenyl]-piperazin-1-yl}-2-phenyl-acetamide

To a solution of 157 mg of the product of Step C above in 5 mL of CH₃CN was added Et₃N (0.061 mL) followed by acetyl chloride (0.029 mL). After 20 min at room temperature Burgess reagent (175 mg) was added and the mixture heated at 105° C. for 18 h. The mixture was concentrated in vacuo and chromatographed on 40 g of SiO₂ (CH₂Cl₂ to 8% 2M NH₃ in MeOH/CH₂Cl₂) to afford 73.4 mg of the title compound as a pale yellow oil.

MS (electrospray): exact mass for C₂₅H₃₀FN₅O₂, 451.24; found m/z 452.3 [M+H]⁺

¹H NMR (500 MHz, CDCl₃): 7.69 (dd, J=8.43, 1.85 Hz, 1H), 7.64 (dd, J=13.36, 1.92 Hz, 1H), 7.47-7.43 (m, 2H), 7.39-7.30 (m, 3H), 6.98-6.92 (m, 1H), 4.26 (s, 1H), 3.52-3.34 (m, 2H), 3.32-3.13 (m, 6H), 2.78-2.64 (m, 4H), 2.59 (s, 3H), 1.12-1.02 (m, 6H).

### Example 24

### 3-{4-[4-(Diethylcarbamoyl-phenyl-methyl)-piperazin-1-yl]-3-fluorophenyl}-[1,2,4]oxadiazole-5-carboxylic acid (Compound #151)

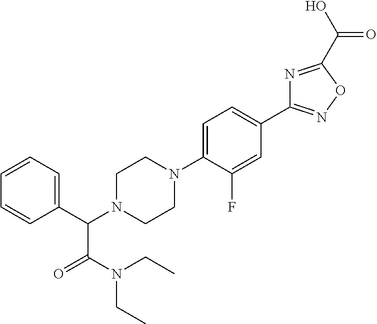

### Step A: 3-{4-[4-(Diethylcarbamoyl-phenyl-methyl)-piperazin-1-yl]-3-fluoro-phenyl}-[1,2,4]oxadiazole-5-carboxylic acid

To a heterogeneous mixture of the product prepared as in Example 18 (109 mg) in EtOH (5 mL) was added LiOH (10 mg). The resulting mixture was stirred at room temperature for 60 h and then concentrated in vacuo. Chromatography by reverse phase HPLC yielded the title compound as a white solid.

MS (electrospray): exact mass for C₂₅H₂₈FN₅O₄, 481.21; found m/z 482.3 [M+H]⁺

¹H NMR (500 MHz, CDCl₃): 7.56-7.04 (m, 6H), 3.54-2.17 (m, 13H), 1.14-0.65 (m, 8H).

### Example 25

### N,N-Diethyl-2-{4-[4-(5-ethyl-[1,3,4]oxadiazol-2-yl)-2-fluoro-phenyl]-piperazin-1-yl}-2-phenyl-acetamide (Compound #152)

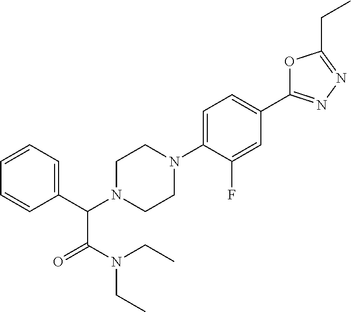

N,N-Diethyl-2-{4-[4-(5-ethyl-[1,3,4]oxadiazol-2-yl)-2-fluoro-phenyl]-piperazin-1-yl}-2-phenyl-acetamide was prepared according to the procedure as described in Example 23, Step D utilizing 148 mg of the product of Example 23, Step C and 0.033 mL of propionyl chloride to yield the title compound as colorless oil.

MS (electrospray): exact mass for C₂₆H₃₂FN₅O₂, 465.25; found m/z 466.3 [M+H]⁺

¹H NMR (500 MHz, CDCl₃): 7.70 (dd, J=8.45, 1.90 Hz, 1H), 7.64 (dd, J=13.35, 1.86 Hz, 1H), 7.47-7.43 (m, 2H), 7.39-7.30 (m, 3H), 6.98-6.92 (m, 1H), 4.27 (s, 1H), 3.52-3.35 (m, 2H), 3.33-3.14 (m, 6H), 2.92 (q, J=7.59 Hz, 2H), 2.78-2.64 (m, 4H), 1.42 (t, J=7.59 Hz, 3H), 1.12-1.01 (m, 6H).

### Example 26

### N,N-Diethyl-2-{4-[2-fluoro-4-(5-isopropyl-[1,3,4]oxadiazol-2-yl)-phenyl]-piperazin-1-yl}-2-phenyl-acetamide (Compound #154)

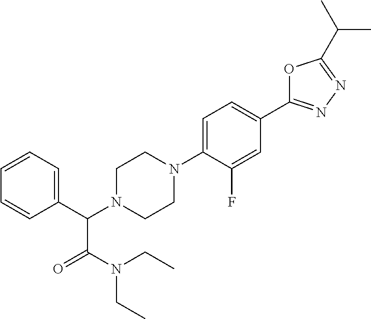

N,N-Diethyl-2-{4-[2-fluoro-4-(5-isopropyl-[1,3,4]oxadiazol-2-yl)-phenyl]-piperazin-1-yl}-2-phenyl-acetamide was prepared according to the procedure as described in Example 23, Step D utilizing 164 mg of the product of Example 23 Step C and isobutyryl chloride (0.045 mL) to yield the title compound as white solid.

MS (electrospray): exact mass for C₂₇H₃₄FN₅O₂, 479.27; found m/z 480.3 [M+H]⁺

¹H NMR (500 MHz, CDCl₃): 7.71 (dd, J=8.45, 1.82 Hz, 1H), 7.64 (dd, J=13.37, 1.95 Hz, 1H), 7.48-7.43 (m, 2H), 7.39-7.30 (m, 3H), 6.98-6.92 (m, 1H), 4.27 (s, 1H), 3.52-3.35 (m, 2H), 3.32-3.13 (m, 7H), 2.79-2.64 (m, 4H), 1.43 (d, J=6.95 Hz, 6H), 1.12-1.02 (m, 6H).

### Example 27

### N,N-Diethyl-2-[4-(2-fluoro-4-[1,2,4]oxadiazol-3-yl-phenyl)-piperazin-1-yl]-2-phenyl-acetamide (Compound #158)

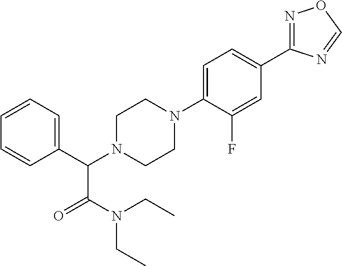

### Step A: 4-(2-Fluoro-[1,2,4]oxadiazol-3-yl-phenyl)-piperazine-1-carboxylic acid tert-butyl ester

To a heterogeneous mixture of the product prepared as in Example 1, Step B (530 mg) in (CH₃O)₃CH (10 mL) was added two drops of BF₃.Et₂O. The resulting mixture was heated to 110° C., during which time it became homogeneous, for 30 min. The resulting mixture was washed with saturated aqueous NaHCO₃ solution and the aqueous layer was extracted with CH₂Cl₂. The combined organic layers were dried over Na₂SO₄, filtered and concentrated in vacuo. The resulting residue was chromatographed on SiO₂ (Hexanes to 25% EtOAc/Hexanes) to yield the title compound as a white solid.

MS (electrospray): exact mass for C₁₇H₂₁FN₄O₃, 348.16; found m/z 293.3 [M-^{t}Bu]⁺, 371.2 [M+Na]⁺.

### Step B: N,N-Diethyl-2-[4-(2-fluoro-[1,2,4]oxadiazol-3-yl-phenyl)-piperazin-1-yl]-2-phenyl-acetamide

N,N-Diethyl-2-[4-(2-fluoro-4-[1,2,4]oxadiazol-3-yl-phenyl)-piperazin-1-yl]-2-phenyl-acetamide was prepared according to the procedure as described in Example 1, Step D reacting the product prepared as in Step A above, stirred at room temperature for 48 h to yield the title compound as a colorless foam.

MS (electrospray): exact mass for C₂₄H₂₈FN₅O₂, 437.22; found m/z 438.3 [M+H]⁺

¹H NMR (500 MHz, CDCl₃): 8.70 (s, 1H), 7.80 (dd, J=8.39, 1.75 Hz, 1H), 7.73 (dd, J=13.51, 1.94 Hz, 1H), 7.48-7.43 (m, 2H), 7.39-7.30 (m, 3H), 7.00-6.94 (m, 1H), 4.27 (s, 1H), 3.51-3.36 (m, 2H), 3.33-3.15 (m, 6H), 2.78-2.65 (m, 4H), 1.12-1.02 (m, 6H).

### Example 28

### N,N-Diethyl-2-{4-[2-fluoro-4-(4-methyl-5-oxo-4,5-dihydro-[1,2,4]oxadiazol-3-yl)-phenyl]-piperazin-1-yl}-2-phenyl-acetamide (Compound #176)

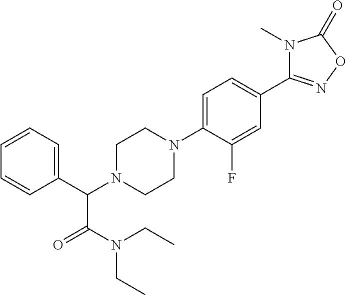

### Step A: 4-[2-Fluoro-4-(5-oxo-4,5-dihydro-[1,2,4]oxadiazol-3-yl)-phenyl]-piperazine-1-carboxylic acid tert-butyl ester

To a heterogeneous mixture of the product prepared as in Example 1, Step B (800 mg) in 1,4-dioxane (5 mL) was added CU (460 mg). The resulting mixture was heated at 110° C. for 30 min during which time it became homogeneous. The resulting mixture was concentrated in vacuo and chromatographed on SiO₂ (25% EtOAc/Hexanes to 100% EtOAc) to yield the title compound as a white solid.

MS (electrospray): exact mass for C₁₇H₂₁FN₄O₄, 364.15; found m/z 265.2 [M-Boc]⁺, 309.2 [M-^{t}Bu]⁺, 387.2 [M+Na]⁺.

### Step B: 4-[2-Fluoro-4-(4-methyl-5-oxo-4,5-dihydro-[1,2,4]oxadiazol-3-yl)-phenyl]-piperazine-1-carboxylic acid tert-butyl ester

4-[2-Fluoro-4-(4-methyl-5-oxo-4,5-dihydro-[1,2,4]oxadiazol-3-yl)-phenyl]-piperazine-1-carboxylic acid tert-butyl ester was prepared according to the procedure as described in Example 23, Step A reacting 534 mg of the product prepared as in Step A above. Chromatography on SiO₂ (Hexanes to 50% EtOAc/Hexanes) yielded the title compound as a white solid.

MS (electrospray): exact mass for C₁₈H₂₃FN₄O₄, 378.17; found m/z 279.2 [M-Boc]⁺, 323.2 [M-^{t}Bu]⁺, 401.2 [M+Na]⁺.

### Step C: N,N-Diethyl-2-{4-[2-fluoro-4-(4-methyl-5-oxo-4,5-dihydro-[1,2,4]oxadiazol-3-yl)-phenyl]-piperazin-1-yl}-2-phenyl-acetamide

N,N-Diethyl-2-{4-[2-fluoro-4-(4-methyl-5-oxo-4,5-dihydro-[1,2,4]oxadiazol-3-yl)-phenyl]-piperazin-1-yl}-2-phenyl-acetamide was prepared according to the procedure as described in Example 27, Step B reacting 108 mg of the product prepared as in Step B above to yield the title compound as a residue. The residue was dissolved in Et₂O and treated with excess 1 M HCl in Et₂O. After 30 min, the resulting mixture was concentrated in vacuo to yield the title compound as it corresponding HCl salt, as a white solid.

MS (electrospray): exact mass for C₂₅H₃₀FN₅O₃, 467.23; found m/z 468.3 [M+H]⁺

¹H NMR (500 MHz, MeOH-d₄): 7.69-7.63 (m, 2H), 7.61-7.55 (m, 3H), 7.53-7.47 (m, 2H), 7.29-7.23 (m, 1H), 5.57 (s, 1H), 3.98-3.74 (m, 2H), 3.73-3.58 (m, 1H), 3.57-3.11 (m, 10H), 3.00-2.82 (m, 1H), 1.15-1.08 (m, 3H), 0.90-0.84 (m, 3H).

### Example 30

### N,N-Diethyl-2-{4-[2-fluoro-4-(5-methylsulfanyl-[1,2,4]oxadiazol-3-yl)-phenyl]-piperazin-1-yl}-2-phenyl-acetamide (Compound #177)

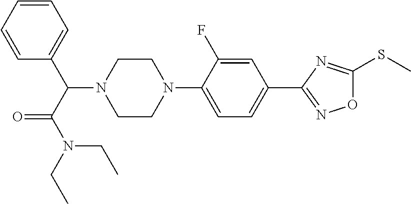

### Step A: 4-[2-Fluoro-4-(5-thioxo-4,5-dihydro-[1,2,4]oxadiazol-3-yl)-phenyl]-piperazine-1-carboxylic acid tert-butyl ester

To a solution of the product prepared as in Example 1, Step B (528 mg) in 1,4-dioxane (10 mL) was added TCDI (292 mg) followed by DBU (0.245 mL). The resulting mixture was heated at 80° C. for 90 min and then concentrated in vacuo to yield a residue. The residue was chromatographed on SiO₂(25% EtOAc/Hexanes to 100% EtOAc) to yield the title compound as a semi-pure yellow solid.

### Step B: 4-[2-Fluoro-4-(5-methylsulfanyl-[1,2,4]oxadiazol-3-yl)-phenyl]-piperazine-1-carboxylic acid tert-butyl ester

4-[2-Fluoro-4-(5-methylsulfanyl-[1,2,4]oxadiazol-3-yl)-phenyl]-piperazine-1-carboxylic acid tert-butyl ester was prepared according to the procedure as described in Example 23, Step A reacting 125 mg of the product prepared as in Step A above. Chromatography on SiO₂ (Hexanes to 20% EtOAc/Hexanes) yielded the title compound as a white solid.

MS (electrospray): exact mass for C₁₈H₂₃FN₄O₃S, 394.15; found m/z 294.2 [M-Boc]⁺, 339.2 [M-^{t}Bu]⁺, 417.2 [M+Na]⁺.

### Step C: N,N-Diethyl-2-{4-[2-fluoro-4-(5-methylsulfanyl-[1,2,4]oxadiazol-3-yl)-phenyl]-piperazin-1-yl}-2-phenyl-acetamide

N,N-Diethyl-2-{4-[2-fluoro-4-(5-methylsulfanyl-[1,2,4]oxadiazol-3-yl)-phenyl]-piperazin-1-yl}-2-phenyl-acetamide was prepared according to the procedure as described in Example 27, Step B reacting 70 mg of the product prepared as in Step B above to yield the title compound as a white solid.

MS (electrospray): exact mass for C₂₅H₃₀FN₅O₂S, 483.21; found m/z 484.3 [M+H]⁺

¹H NMR (500 MHz, CDCl₃): 7.73 (dd, J=8.40, 1.74 Hz, 1H), 7.67 (dd, J=13.55, 1.92 Hz, 1H), 7.48-7.43 (m, 2H), 7.39-7.29 (m, 3H), 6.98-6.91 (m, 1H), 4.26 (s, 1H), 3.52-3.36 (m, 2H), 3.33-3.14 (m, 6H), 2.79-2.64 (m, 7H), 1.12-1.01 (m, 6H).

### Example 31

### 2-{4-[4-(3,5-Dimethyl-isoxazol-4-yl)-2-fluoro-phenyl]-piperazin-1-yl}-N,N-diethyl-2-phenyl-acetamide (Compound #178)

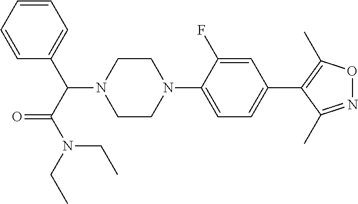

The title compound was prepared according to procedure as described in Example 44 which follows herein, with the appropriate substitutions.

MS (ESI): mass calcd. for C₂₇H₃₃FN₄O₂, 464.58; m/z found, 465.3 [M+H]⁺

¹H NMR (CDCl₃): 7.49-7.45 (m, 2H), 7.41-7.31 (m, 3H), 6.98-6.87 (m, 3H), 4.26 (br s, 1H), 3.52-3.34 (m, 2H), 3.33-3.12 (m, 6H), 2.82-2.63 (br m, 4H), 2.38 (s, 3H), 2.25 (s, 3H), 1.10 (t, J=7.1 Hz, 3H), 1.05 (t, J=7.1 Hz, 3H)

### Example 31

### 2-{4-[2-Fluoro-4-(3-isopropyl-[1,2,4]oxadiazol-5-yl)-phenyl]-piperazin-1-yl}-N,N-dimethyl-2-phenyl-acetamide (Compound #180)

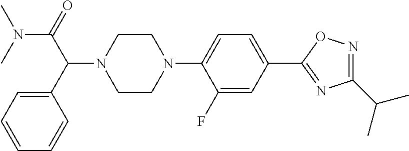

The title compound was prepared according to the process outlined in Example 6 above, with the appropriate substituent changes.

MS (ESI): mass calcd. for C₂₅H₃₀FN₅O₂, 451.2; m/z found, 452.3 [M+H]⁺

¹H NMR (CDCl₃): 7.83-7.70 (m, 2H), 7.48-7.30 (m, 5H), 6.96 (t, J=8.6, 1H), 4.32 (s, 1H), 3.35-3.20 (m, 4H), 3.18-3.11 (m, 1H), 2.98 (s, 3H), 2.96 (s, 3H), 2.75-2.63 (m, 4H), 1.37 (d, J=6.9, 6H).

### Example 32

### 2-{4-[4-(3-Ethyl-[1,2,4]oxadiazol-5-yl)-2-fluoro-phenyl]-piperazin-1-yl}-N,N-dimethyl-2-phenyl-acetamide (Compound #181)

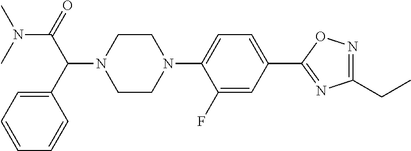

The title compound was prepared according to the process outlined in Example 6 above, with the appropriate substituent changes.

MS (ESI): mass calcd. for C₂₄H₂₈FN₅O₂, 437.2; m/z found, 438.2 [M+H]⁺

¹H NMR (CDCl₃): 7.84-7.70 (m, 2H), 7.48-7.30 (m, 5H), 6.96 (t, J=8.5, 1H), 4.32 (s, 1H), 3.32-3.22 (m, 4H), 3.01 (s, 3H), 2.96 (s, 3H), 2.85-2.64 (m, 6H), 1.40-1.36 (m, 3H),

### Example 33

### 2-{4-[2-Fluoro-4-(3-methyl-[1,2,4]oxadiazol-5-yl)-phenyl]-piperazin-1-yl}-N,N-dimethyl-2-phenyl-acetamide (Compound #182)

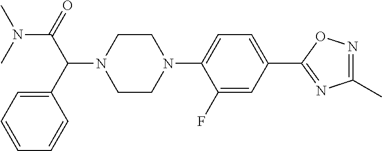

The title compound was prepared according to the process outlined in Example 6 above, with the appropriate substituent changes.

MS (ESI): mass calcd. for C₂₃H₂₆FN₅O₂, 423.2; m/z found, 424.3 [M+H]⁺

¹H NMR (CDCl₃): 7.83-7.70 (m, 2H), 7.55-7.38 (m, 5H), 6.96 (t, J=8.6, 1H), 4.32 (s, 1H), 3.40-3.30 (br, 4H), 3.02-2.74 (m, 10H), 2.46 (s, 3H).

### Example 34

### {4-[2-Fluoro-4-(3-methyl-[1,2,4]oxadiazol-5-yl)-phenyl]-piperazin-1-yl}-phenyl-acetic acid methyl ester (Compound #183)

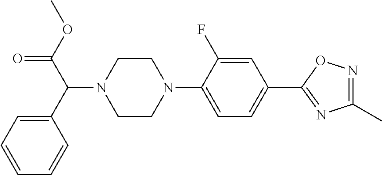

### Step A: {4-[2-Fluoro-4-(3-methyl-[1,2,4]oxadiazol-5-yl)-phenyl]-piperazin-1-yl}-phenyl-acetic acid

To a solution of the product prepared as in Example 71 (952 mg) which follows herein, in CH₂Cl₂ (5 mL) was treated with TFA (20 mL). After 6 h the resulting mixture was concentrated in vacuo to yield the title compound.

MS (electrospray): exact mass for C₂₁H₂₁FN₄O₃, 410.18; found m/z 397.2 [M+H]⁺.

### Step B: {4-[2-Fluoro-4-(3-methyl-[1,2,4]oxadiazol-5-yl)-phenyl]-piperazin-1-yl}-phenyl-acetic acid methyl ester

{4-[2-Fluoro-4-(3-methyl-[1,2,4]oxadiazol-5-yl)-phenyl]-piperazin-1-yl}-phenyl-acetic acid methyl ester was prepared according to the procedure as described in Example 23, Step A reacting 832 mg of the product prepared as in Step A above to yield a residue. The residue was chromatographed on SiO₂ (Hexanes to 40% EtOAc/Hexanes) to yield the title compound as a yellow solid.

MS (electrospray): exact mass for C₂₂H₂₃FN₄O₃, 410.18; found m/z 411.3 [M+H]⁺

¹H NMR (500 MHz, CDCl₃): 7.80 (dd, J=8.39, 1.89 Hz, 1H), 7.72 (dd, J=13.38, 1.96 Hz, 1H), 7.50-7.43 (m, 2H), 7.40-7.31 (m, 3H), 7.00-6.93 (m, 1H), 4.09 (s, 1H), 3.71 (s, 3H), 3.32-3.24 (m, 4H), 2.70-2.62 (m, 4H), 2.44 (s, 3H).

### Example 35

### N-Ethyl-2-{4-[2-fluoro-4-(3-isopropyl-[1,2,4]oxadiazol-5-yl)-phenyl]-piperazin-1-yl}-N-methyl-2-phenyl-acetamide (Compound #184)

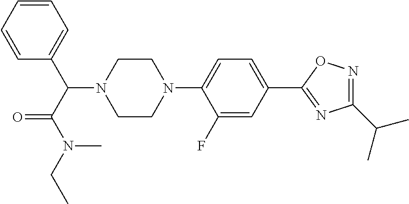

The title compound was prepared according to the procedure as described in Example 6, with the appropriate substituent changes.

MS (ESI): mass calcd. for C₂₆H₃₂FN₅O₂, 465.25; m/z found, 466.3 [M+H]⁺

¹H NMR (CDCl₃): 7.83-7.70 (m, 2H), 7.48-7.30 (m, 5H), 6.96 (t, J=8.6, 1H), 4.28 (d, J=6.9, 1H), 3.56-3.08 (m, 7H), 2.91 (s, 3H), 2.78-2.62 (m, 4H), 1.37 (d, J=7.0, 6H), 1.10-0.98 (m, 3H).

### Example 36

### 2-{4-[2-Fluoro-4-(3-isopropyl-[1,2,4]oxadiazol-5-yl)-phenyl]-piperazin-1-yl}-2-phenyl-1-pyrrolidin-1-yl-ethanone (Compound #185)

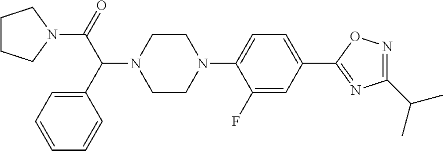

The title compound was prepared according to the process outlined in Example 6 above, with the appropriate substituent changes.

MS (ESI): mass calcd. for C₂₇H₃₂FN₅O₂, 477.3; m/z found, 478.3 [M+H]⁺

¹H NMR (CDCl₃): 7.83-7.70 (m, 2H), 7.48-7.30 (m, 5H), 6.96 (t, J=8.6, 1H), 4.13 (s, 1H), 3.63-3.40 (m, 3H), 3.35-3.20 (m, 5H), 3.18-3.10 (m, 1H), 2.75-2.63 (m, 4H), 1.98-1.70 (m, 4H), 1.37 (d, J=7.0, 6H).

### Example 37

### N,N-Diethyl-2-{4-[2-fluoro-4-(5-methyl-2-phenyl-2H-pyrazol-3-yl)-phenyl]-piperazin-1-yl}-2-phenyl-acetamide (Compound #186)

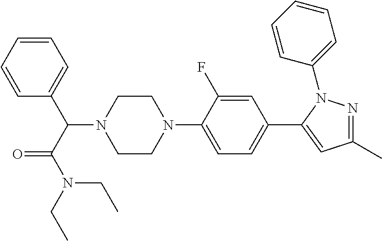

The title compound was prepared according to the procedure as described in Example 44 which follows herein, with the appropriate substitutions.

MS (ESI): mass calcd. for C₃₂H₃₆FN₅O, 525.67; m/z found, 526.3 [M+H]⁺.

¹H NMR (CDCl₃): 7.45-7.42 (m, 2H), 7.37-7.14 (m, 10H), 6.89-6.75 (m, 3H), 6.24 (s, 1H), 4.23 (br s, 1H), 3.50-3.34 (m, 2H), 3.31-3.08 (m, 6H), 2.74-2.61 (m, 4H), 2.36 (s, 3H), 1.10-1.00 (m, 6H)

### Example 38

### 2-{4-[4-(Diethylcarbamoyl-phenyl-methyl)-piperazin-1-yl]-3-fluoro-phenyl}-pyrrole-1-carboxylic acid tert-butyl ester (Compound #187)

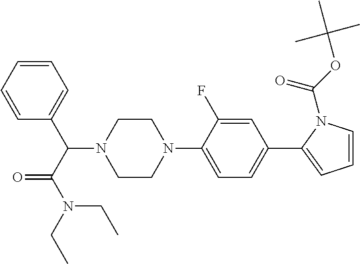

The title compound was prepared according to the procedure as described in Example 44 which follows herein, with the appropriate substitutions.

MS (ESI): mass calcd. for C₃₁H₃₉FN₄O₃, 534.67; m/z found, 535.3 [M+H]⁺.'

¹H NMR (CDCl3): 7.48-7.43 (m, 2H), 7.38-7.29 (m, 4H), 7.03-6.97 (m, 2H), 6.91-6.86 (m, 1H), 6.20-6.18 (m, 1H), 6.15-6.12 (m, 1H), 4.24 (s, 1H), 3.51-3.35 (m, 2H), 3.33-3.06 (m, 6H), 2.76-2.63 (m, 4H), 1.37 (s, 9H), 1.11-1.02 (m, 6H)

### Example 39

### N,N-Diethyl-2-{4-[2-fluoro-4-(1H-pyrrol-2-yl)-phenyl]-piperazin-1-yl}-2-phenyl-acetamide (Compound #188)

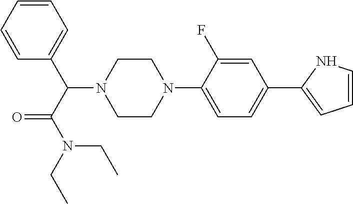

To a solution of 2-{4-[4-(diethylcarbamoyl-phenyl-methyl)-piperazin-1-yl]-3-fluoro-phenyl}-pyrrole-1-carboxylic acid tert-butyl ester (167.0 mg, 0.31 mmol) prepared as in Example 38, in methanol (5 mL) was added 1M NaOH (2 mL). The reaction mixture was heated to 60° C. for 2 h. The resulting mixture was cooled and solvent removed. The residue was partitioned between CH₂Cl₂ and water. The aqueous was extracted with CH₂Cl₂ and the solvent removed from the combined organic layers. The residue was purified on silica gel (40 g, Hexanes/EtOAc 0-50%) to yield the title compound as a thick oil.

MS (ESI): mass calcd. for C₂₆H₃₁FN₄O, 434.56; m/z found, 435.3 [M+H]⁺.

¹H NMR (CDCl₃): 8.42 (br s, 1H), 7.48-7.44 (m, 2H), 7.38-7.29 (m, 3H), 7.16-7.10 (m, 2H), 6.93-6.88 (m, 1H), 6.84-6.81 (m, 1H), 6.42-6.39 (m, 1H), 6.28-6.25 (m, 1H), 4.24 (s, 1H), 3.51-3.38 (m, 2H), 3.33-3.09 (m, 6H), 2.75-2.64 (m, 4H), 1.12-1.03 (m, 6H)

### Example 40

### 1-(4,4-Difluoro-piperidin-1-yl)-2-{4-[2-fluoro-4-(3-methyl-[1,2,4]oxadiazol-5-yl)-phenyl]-piperazin-1-yl}-2-phenyl-ethanone (Compound #189)

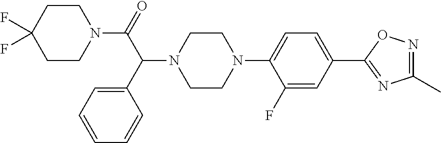

A mixture of 1-[2-Fluoro-4-(3-methyl-[1,2,4]oxadiazol-5-yl)-phenyl]-piperazine (89.5 mg, 0.3 mmol), 2-bromo-1-(4,4-difluoro-piperidin-1-yl)-2-phenyl-ethanone (160 mg, 0.5 mmol), Cs₂CO₃ (294 mg, 0.9 mmol) and KI (10 mg) in acetonitrile was heated to 50° C. for 18 h. The resulting mixture was then cooled to room temperature and partitioned between DCM and water. The organic phase was concentrated to dryness to yield a residue. Chromatography of the residue (SiO₂, 0-50% ethyl acetate-hexane, gradient) yielded title compound.

MS (ESI) mass calcd. for C₂₆H₂₈F₃N₅O₂, 499.53; m/z found, 500.3 [M+H]⁺

¹H NMR (CDCl₃): 7.81-7.77 (dd, J=8.4, 2.0 1H), 7.74-7.69 (dd, J=13.4, 2.0, 1H), 7.43-7.33 (m, 5H), 6.95 (t, J=8.6, 1H), 4.36 (s, 1H), 3.97-3.84 (m, 1H), 3.69-3.49 (m, 3H), 3.37-3.17 (m, 4H), 2.83-2.72 (m, 2H), 2.71-2.62 (m, 4H), 2.44 (s, 3H), 2.00-1.74 (m, 2H).

### Example 41

### N-Ethyl-2-{4-[2-fluoro-4-(3-methyl-[1,2,4]oxadiazol-5-yl)-phenyl]-piperazin-1-yl}-2-phenyl-acetamide (Compound #190)

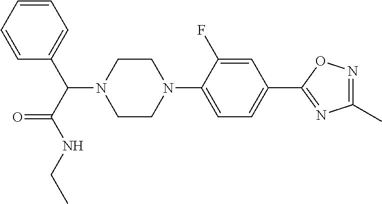

The title compound was prepared according to the process described in Example 40 substituting the appropriate reactants and purifying the isolated residue by chromatography (SiO₂, O-5% ethyl acetate-hexanes, gradient) to yield title compound.

MS (ESI) mass calcd. for C₂₃H₂₆FN₅O₂, 423.28; m/z found, 424.3 [M+H]⁺

¹H NMR (CDCl₃): 7.92-7.78 (dd, J=8.4, 2.0, 1H), 7.75-7.70 (dd, J=13.4, 2.0, 1H), 2.38-2.30 (m, 5H), 7.04-6.93 (m, 2H), 3.89 (s, 1H), 3.40-3.31 (m, 2H), 3.28-3.23 (m, 4H), 2.65-2.59 (m, 4H), 2.44 (s, 3H), 1.17 (t, J=7.2, 3H).

### Example 42

### N,N-Diethyl-2-{4-[2-fluoro-4-(1-isopropyl-5-oxo-4,5-dihydro-1H-pyrazol-3-yl)-phenyl]-piperazin-1-yl}-2-phenyl-acetamide (Compound #191)

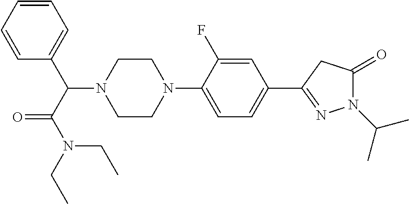

### Step A 4-(2-Fluoro-4-formyl-phenyl)-piperazine-1-carboxylic acid tert-butyl ester

A solution of piperazine-1-carboxylic acid tert-butyl ester, 3,4-difluoro-benzaldehyde and triethyl amine in acetonitrile were refluxed for 48 h. The solvent was removed and the residue partitioned between CH₂Cl₂ and water. The layers were separated and the aqueous layer was extracted with CH₂Cl₂. The organic extracts were combined, dried over Na₂SO₄ and the solvent removed. The residue was purified on silica gel (120 g, Hexanes/EtOAc 0-20%) to yield the title compound as a yellow solid.

¹H NMR (CDCl3): 9.84-9.83 (m, 1H), 7.60-7.57 (m, 1H), 7.56-7.52 (m, 1H), 7.00-6.96 (m, 1H), 3.63-3.59 (m, 4H), 3.23-3.19 (m, 4H), 1.49 (s, 9H).

### Step B. N,N-Diethyl-2-[4-(2-fluoro-4-formyl-phenyl)-piperazin-1-yl]-2-phenyl-acetamide

To a CH₂Cl₂ (10 mL) solution of 4-(2-fluoro-4-formyl-phenyl)-piperazine-1-carboxylic acid tert-butyl ester (0.97 g, 3.14 mmol) prepared as in STEP A above was added TFA (2 mL). The resulting mixture was stirred at ambient temperature for 16 h then the solvent was removed. The residue was dissolved in DMF (10 mL) and K₂CO₃ (2.6 g) added followed by 2-bromo-N,N-diethyl-2-phenyl-acetamide (0.95 g). The resulting mixture was stirred at room temperature for 16 h then diluted with water (50 mL). The resulting mixture was extracted with diethyl ether and the solvent removed from the combined organic extracts. The residue was purified on silica gel (40 g, Hexanes/EtOAc 0-75%) to yield the title compound.

¹H NMR (CDCl3) 9.81-9.80 (m, 1H), 7.56-7.53 (m, 1H), 7.51-7.47 (m, 1H), 7.46-7.43 (m, 2H), 7.39-7.30 (m, 3H), 6.96-6.91 (m, 1H), 4.27 (s, 1H), 3.52-3.44 (m, 1H), 3.42-3.34 (m, 1H), 3.33-3.23 (m, 5H), 3.22-3.13 (m, 1H), 2.78-2.65 (m, 4H), 1.10 (t, J=7.1 Hz, 3H), 1.04 (t, J=7.1 Hz, 3H).

### Step C. N,N-Diethyl-2-{4-[2-fluoro-4-(1-isopropyl-5-oxo-4,5-dihydro-1H-pyrazol-3-yl)-phenyl]-piperazin-1-yl}-2-phenyl-acetamide

To a solution of N,N-diethyl-2-[4-(2-fluoro-4-formyl-phenyl)-piperazin-1-yl]-2-phenyl-acetamide (0.55 g, 1.38 mmol) prepared as in STEP B above in diethyl ether (35 mL) cooled to 0° C. was added BF₃ (0.25 mL) and ethyl diazoacetate (0.20 mL). The resulting mixture was stirred at 0° C. for 3 h (bubbling observed) then quenched with NaHCO₃ (aq) stirred for 1 h. The resulting mixture was extracted with CH₂Cl₂, dried over NaSO4 and the solvent removed. The residue dissolved in EtOH (10 mL) and isopropyl-hydrazine (195.4 mg) added. The resulting mixture was refluxed for 6 h then cooled and solvent removed. The residue purified on silica gel (40 g, EtOAc/Methanol 5%) to yield the title compound.

MS (ESI): mass calcd. for C28H36FN5O2, 493.62; m/z found, 494.3 [M+H]⁺.

¹H NMR (CDCl3): 7.64 (s, 1H), 7.50-7.44 (m, 2H), 7.43-7.30 (m, 4H), 7.13-7.06 (m, 1H), 6.96-6.90 (m, 1H), 4.59-4.49 (m, 1H), 4.28 (br s, 1H), 3.53-3.37 (m, 2H), 3.35-3.08 (m, 6H), 2.81-2.65 (m, 4H), 1.57-1.52 (m, 6H), 1.13-1.03 (m, 6H).

### Example 43

### {4-[2-Fluoro-4-(3-methyl-[1,2,4]oxadiazol-5-yl)-phenyl]-piperazin-1-yl}-phenyl-acetic acid ethyl ester (Compound #192)

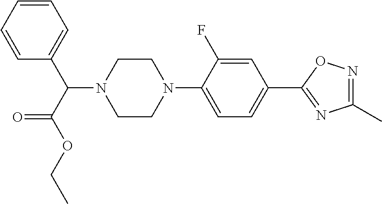

To a solution of 1-[2-fluoro-4-(3-methyl-[1,2,4]oxadiazol-5-yl)-phenyl]-piperazine (356.9 mg, 1.36 mmol) in DMF (5 mL) was added K₂CO₃ (0.80 g) added followed by bromo-phenyl-acetic acid ethyl ester (0.45 g). The resulting mixture was stirred at room temperature for 16 h then diluted with water (50 mL). The resulting mixture was extracted with diethyl ether and the solvent removed from the combined organic extracts. The residue was purified on silica gel (40 g, Hexanes/EtOAc 0-75%) to yield the title compound as a yellow solid.

MS (ESI): mass calcd. for C₂₃H₂₅FN₄O₃, 424.47; m/z found, 425.3 [M+H]⁺

¹H NMR (CDCl3): 7.82-7.78 (m, 1H), 7.75-7.69 (m, 1H), 7.49-7.44 (m, 2H), 7.40-7.31 (m 3H), 6.99-6.93 (m, 1H), 4.26-4.09 (m, 2H), 4.06 (s, 1H), 3.31-3.23 (m, 4H), 2.70-2.62 (m, 4H), 2.44 (s, 3H), 1.22 (t, J=7.1 Hz, 3H)

### Example 44

### N,N-Diethyl-2-[4-(3-fluoro-biphenyl-4-yl)-piperazin-1-yl]-2-phenyl-acetamide (Compound #193)

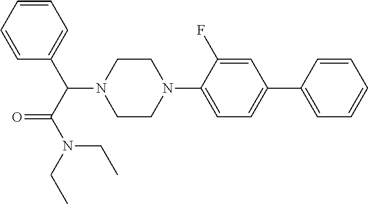

### Step A. 1-[4-(4-Bromo-2-fluoro-phenyl)-piperazin-1-yl]-2,2,2-trifluoro-ethanone

To a solution of 1-(2-fluoro-phenyl)-piperazine (5 mL, 31.6 mmol) in CH₂Cl₂ (50 mL) was added triethylamine (7 mL) and the flask cooled to 0° C. Trifluoroacetic anhydride (5 mL) then added and the reaction stirred at 0° C. for 6 h. Water was then added (100 mL) and 1M HCl added to ˜pH 1. The resulting mixture was extracted with CH₂Cl₂ and the combined organics dried over MgSO₄. The solvent was removed to yield an yellow/orange oil. The oil material (7.81 g) was dissolved in AcOH (100 mL) and cooled to 0° C. Bromine (1.7 mL) was then added slowly. Solids were observed to form quickly and the suspension is stirred vigorously during bromine addition. The resulting creamy orange/yellow suspension was stirred at 0° C. for 1 h before being poured into 1.5 L of water. Sodium metabisulfite was then added to de-color. The resulting mixture was extracted with CH₂Cl₂ and the solvent removed to yield a residue. The residue was purified on silica gel (330 g, Hexanes/EtOAc 0-20%) to yield the title compound.

### Step B. 2-[4-(4-Bromo-2-fluoro-phenyl)-piperazin-1-yl]-N,N-diethyl-2-phenyl-acetamide

To a solution of 1-[4-(4-bromo-2-fluoro-phenyl)-piperazin-1-yl]-2,2,2-trifluoro-ethanone (8.01 g, 22.5 mmol) prepared as in STEP A above in methanol (100 mL) was added potassium carbonate (8.1 g). The resulting mixture was stirred at room temperature for 16 h. The methanol was then removed, water added (400 mL) and the product extracted with CH₂Cl₂. The combined organic layers were dried over Na₂SO₄ and the solvent was removed to yield an off-white solid. The solid (2.39 g, 9.22 mmol) was dissolved in DMF (15 mL) and K₂CO₃ (5.5 g) added followed by 2-bromo-N,N-diethyl-2-phenyl-acetamide (3.0 g). The resulting mixture was stirred at room temperature for 16 h then diluted with water (100 mL). The resulting mixture was extracted with diethyl ether and the solvent removed from the combined organic extracts. The residue was purified on silica gel (120 g, Hexanes/EtOAc 0-75%) to yield the title compound.

MS (ESI): mass calcd. for C₂₂H₂₇BrFN₃O, 448.38; m/z found, 449.2 [M+H]⁺.

¹H NMR (CDCl3): 7.46-7.42 (m, 2H), 7.38-7.29 (m, 3H), 7.17-7.12 (m, 2H), 6.80-6.75 (m, 1H), 4.23 (s, 1H), 3.51-3.35 (m, 2H), 3.32-3.14 (m, 2H), 3.13-3.04 (m, 4H), 2.74-2.62 (m, 4H), 1.08 (t, J=7.1 Hz, 3H), 1.04 (t, J=7.1 Hz, 3H) borate ester: (CDCl₃): 7.50-7.29 (m, 7H), 6.91-6.85 (m, 1H), 4.23 (s, 1H), 3.50-3.36 (m, 2H), 3.33-3.12 (m, 6H), 2.76-2.62 (m, 4H), 1.31 (s, 12H), 1.11-1.01 (m, 6H)

### Step C. N,N-Diethyl-2-[4-(3-fluoro-biphenyl-4-yl)-piperazin-1-yl]-2-phenyl-acetamide

A mixture of 2-[4-(4-bromo-2-fluoro-phenyl)-piperazin-1-yl]-N,N-diethyl-2-phenyl-acetamide (158.5 mg, 0.35 mmol), phenyl boronic acid (0.15 g), potassium phosphate (0.22 g) and PdCl₂dppf (9.2 mg) in 1,4-dioxane (3 mL) was heated to 100° C. for 16 h. The resulting mixture cooled, solvent removed and the residue purified on silica gel (40 g, hexanes/EtOAc 0-50%) to yield the title compound as a white foam.

MS (ESI): mass calcd. for C₂₈H₃₂FN₃O, 445.58; m/z found, 446.3 [M+H]⁺.

¹H NMR (CDCl3): 7.54-7.50 (m, 2H), 7.48-7.45 (m, 2H), 7.43-7.23 (m, 8H), 7.00-6.95 (m, 1H), 4.24 (s, 1H), 3.51-3.38 (m, 2H), 3.33-3.13 (m, 6H), 2.76-2.66 (m, 4H), 1.12-1.03 (m, 6H)

### Example 45

### 2-[4-(3′-Chloro-3-fluoro-biphenyl-4-yl)-piperazin-1-yl]-N,N-diethyl-2-phenyl-acetamide (Compound #194)

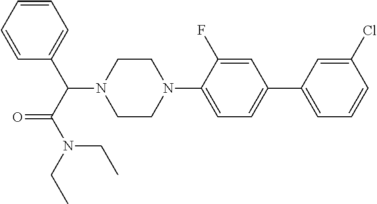

The title compound was prepared according to the procedure as described in Example 44 above, with the appropriate substitutions.

MS (ESI): mass calcd. for C₂₈H₃₁ClFN₃O, 480.02; m/z found, 481.3 [M+H]⁺.

¹H NMR (CDCl3): 7.51-7.44 (m, 3H), 7.41-7.19 (m, 8H), 7.00-6.94 (m, 1H), 4.24 (s, 1H), 3.52-3.36 (m, 2H), 3.34-3.13 (m, 6H), 2.77-2.66 (m, 4H), 1.12-1.02 (m, 6M)

### Example 46

### 2-{4-[2-Fluoro-4-(3-methyl-[1,2,4]oxadiazol-5-yl)-phenyl]-piperazin-1-yl}-2-phenyl-propionic acid ethyl ester (Compound #226)

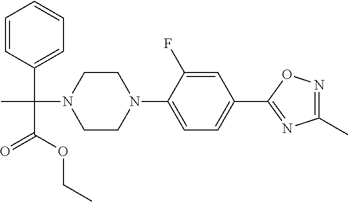

To a THF (4 mL) solution of {4-[2-fluoro-4-(3-methyl-[1,2,4]oxadiazol-5-yl)-phenyl]-piperazin-1-yl}-phenyl-acetic acid ethyl ester (105 mg, 0.25 mmol) prepared as in Example 43, cooled to −78° C., was added NaHMDS (0.35 mL, 1M). The resulting orange solution was stirred at −78° C. for 1 h, then CH₃I (20 μL) was added and the resulting mixture was slowly allowed to warm to room temperature over 3 h. Ammonium chloride (2 mL, aq.) and water (50 mL) were added and the resulting mixture was extracted with diethyl ether. The combined organic layers were dried over Na₂SO₄ and the solvent removed. The residue was purified on silica gel (12 g, Hexanes/EtOAc 0-50%) to yield the title compound.

MS (ESI): mass calcd. for C₂₄H₂₇FN₄O₃, 438.5; m/z found, 439.3 [M+H]⁺

¹H NMR (CDCl3): 7.83-7.78 (m, 1H), 7.75-7.70 (m, 1H), 7.56-7.52 (m, 2H), 7.37-7.25 (m, 3H), 7.01-6.95 (m, 1H), 4.20 (q, J=7.1 Hz, 2H), 3.32-3.20 (m, 4H), 2.82-2.66 (m, 4H), 2.44 (s, 3H), 1.66 (s, 3H), 1.25 (t, J=7.1 Hz, 3H)

### Example 47

### N,N-Diethyl-2-{4-[2-fluoro-4-(1-methyl-1H-pyrazol-4-yl)-phenyl]-piperazin-1-yl}-2-phenyl-acetamide (Compound #195)

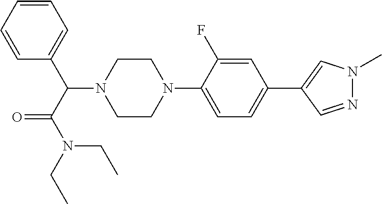

The title compound was prepared according to the procedure as described in Example 44 above, with the appropriate substitutions.

MS (ESI): mass calcd. for C₂₆H₃₂FN₅O, 449.57; m/z found, 450.3 [M+H]⁺.

¹H NMR (CDCl3): 7.67-7.66 (m, 1H), 7.52-7.51 (m, 1H), 7.48-7.44 (m, 2H), 7.38-7.29 (m, 3H), 7.16-7.06 (m, 2H), 6.94-6.87 (m, 1H), 4.23 (s, 1H), 3.92 (s, 3H), 3.51-3.36 (m, 2H), 3.33-3.07 (m, 6H), 2.76-2.63 (m, 4H), 1.12-1.02 (m, 6H)

### Example 48

### 2-[4-(4′-Chloro-3-fluoro-biphenyl-4-yl)-piperazin-1-yl]-N,N-diethyl-2-phenyl-acetamide (Compound #196)

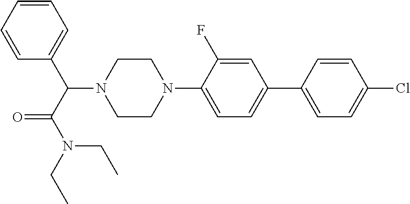

The title compound was prepared according to the procedure as described in Example 44 above, with the appropriate substitutions.

MS (ESI): mass calcd. for C₂₈H₃₁ClFN₃O, 480.02; m/z found, 481.3 [M+H]⁺

¹H NMR (CDCl3): 7.49-7.42 (m, 4H), 7.39-7.30 (m, 5H), 7.26-7.18 (m, 2H), 7.00-6.93 (m, 1H), 4.24 (s, 1H), 3.52-3.36 (m, 2H), 3.33-3.11 (m, 6H), 2.78-2.64 (m, 4H), 1.12-1.02 (m, 6H)

### Example 49

### N-Ethyl-2-{4-[4-(3-ethyl-[1,2,4]oxadiazol-5-yl)-2-fluoro-phenyl]-piperazin-1-yl}-N-methyl-2-phenyl-acetamide (Compound #197)

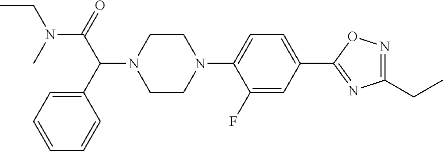

The title compound was prepared according to the process outlined in Example 6 above, with the appropriate substituent changes.

MS (ESI): mass calcd. for C₂₅H₃₀FN₅O₂, 451.2; m/z found, 452.3 [M+H]⁺

¹H NMR (CDCl₃): 7.84-7.71 (m, 2H), 7.50-7.32 (m, 5H), 6.95 (t, J=8.6, 1H), 4.38 (d, J=6.6, 1H), 3.53-3.34 (m, 2H), 3.32-3.18 (m, 4H), 2.91 (s, 3H), 2.85-2.63 (m, 6H), 1.38 (t, J=7.6, 3H), 1.12-1.00 (m, 3H).

### Example 50

### 1-{4-[2-Fluoro-4-(3-isopropyl-[1,2,4]oxadiazol-5-yl)-phenyl]-piperazin-1-yl}-1-phenyl-propan-2-one (compound #198)

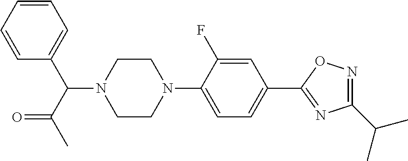

The title compound was prepared according to the process outlined in Example 6 reacting 1-chloro-1-phenyl-propan-2-one with 1-phenyl-propan-2-one (1.1 g, 8.4 mmol) in CCl₄ (3 mL) at 0° C., adding SO₂Cl₂ (0.75 mL, 9.3 mmol). The resulting mixture was stirred at room temperature for 24 h, then concentrated to yield the title compound as a residue. (according to the process as outlined in Example 2 above)

MS (ESI): mass calcd. for C₂₄H₂₇FN₄O₂, 422.2; m/z found, 423.3 [M+H]⁺

¹H NMR (CDCl₃): 7.84-7.73 (m, 2H), 7.48-7.32 (m, 4H), 7.04-6.94 (m, 2H), 4.03 (s, 1H), 3.35-3.20 (m, 4H), 3.18-3.10 (m, 1H), 2.75-2.63 (m, 4H), 2.15-2.12 (s, 3H), 1.43-1.37 (m, 6H).

### Example 51

### 2-{4-[2-Fluoro-4-(3-methyl-[1,2,4]oxadiazol-5-yl)-phenyl]-piperazin-1-yl}-hexanoic acid diethylamide (Compound #199)

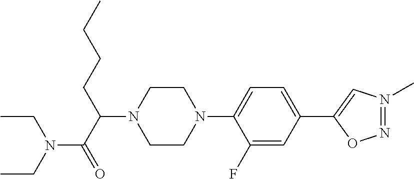

The title compound was prepared according to the process described in Example 40, substituting the appropriate reactants and purifying the isolated residue by chromatography (SiO₂, O-5% 2M NH₃ in MeOH/DCM, gradient) to yield title compound.

MS (ESI) mass calcd. for C₂₃H₃₄FN₅O₂, 431.55; m/z found, 432.3 [M+H]⁺

¹H NMR (CDCl₃): 7.81-7.76 (dd, J=8.5, 1.8, 1H), 7.74-7.68 (dd, J=13.4, 2.0, 1H), 6.95 (t, J=8.6, 1H), 3.60-3.43 (m, 2H), 3.42-3.26 (m, 3H), 3.24-3.18 (m, 4H), 2.92-2.84 (m, 2H), 2.75-2.67 (m, 2H), 2.43 (s, 3H), 1.93-1.80 (m, 1H), 1.67-1.57 (m, 1H), 1.40-1.27 (m, 4H), 1.26-1.24 (m, 1H), 1.21 (t, J=7.1, 4H), 1.12 (t, J=7.0, 4H).

### Example 52

### N,N-Diethyl-2-{4-[2-fluoro-4-(5-methyl-2H-pyrazol-3-yl)-phenyl]-piperazin-1-yl}-2-phenyl-acetamide (Compound 3200)

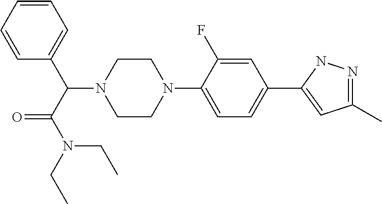

The title compound was prepared according to the procedure as described in Example 44 above, with the appropriate substitutions.

MS (ESI): mass calcd. for C₂₆H₃₂FN₅O, 449.57; m/z found, 450.3 [M+H]⁺

¹H NMR (CDCl₃): 7.49-7.44 (m, 2H), 7.40-7.29 (m, 5H), 6.94-6.88 (m, 1H), 6.25 (s, 1H), 4.24 (s, 1H), 3.51-3.37 (m, 2H), 3.35-3.08 (m, 6H), 2.75-2.64 (m, 4H), 2.32 (s, 3H), 1.11-1.02 (m, 6H)

### Example 53

### N,N-Diethyl-2-[4-(2-fluoro-4-pyridin-2-yl-phenyl)-piperazin-1-yl]-2-phenyl-acetamide (Compound #201)

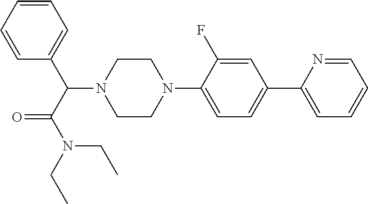

The title compound was prepared according to the process outlined in Example 44 herein, with the appropriate substituent changes.

MS (ESI): mass calcd. for C₂₇H₃₁FN₄O, 446.56; m/z found, 447.3 [M+H]⁺

¹H NMR (CDCl₃): 8.70-8.50 (m, 1H), 7.78-7.56 (m, 4H), 7.53-7.45 (m, 2H), 7.43-7.32 (m, 3H), 7.22-7.11 (m, 1H), 7.00 (t, J=8.8, 1H), 4.28 (bs, 1H), 3.50-3.44 (m, 2H), 3.32-3.28 (m, 1H), 3.26-3.18 (m, 5H), 2.80-2.61 (m, 4H), 1.11 (t, J=7.1, 3H), 1.06 (t, J=7.1, 3H).

### Example 54

### N,N-Diethyl-2-[4-(2-fluoro-4-pyridin-3-yl-phenyl)-piperazin-1-yl]-2-phenyl-acetamide (Compound #202)

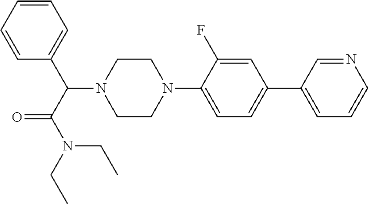

The title compound was prepared according to the procedure as described in Example 44 above, with the appropriate substitutions.

MS (ESI): mass calcd. for C₂₇H₃₁FN₄O, 446.57; m/z found, 447.3 [M+H]⁺

¹H NMR (CDCl₃): 8.80-8.78 (m, 1H), 8.57-8.53 (m, 1H), 7.83-7.78 (m, 1H), 7.50-7.44 (m, 2H), 7.40-7.21 (m, 6H), 7.04-6.97 (m, 1H), 4.26 (s, 1H), 3.53-3.36 (m, 2H), 3.33-3.14 (m, 6H), 2.79-2.66 (m, 4H), 1.13-1.02 (m, 6H)

### Example 55

### 2-{4-[2-Bromo-4-(3-methyl-[1,2,4]oxadiazol-5-yl)-phenyl]-piperazin-1-yl}-N,N-diethyl-2-phenyl-acetamide (compound #203)

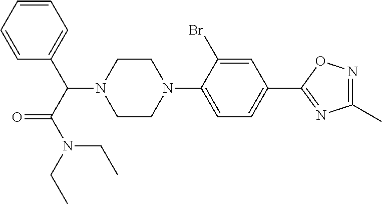

### Step A: 4-(2-Bromo-4-cyano-phenyl)-piperazine-1-carboxylic acid tert-butyl ester

To a solution of 3-bromo-4-fluorobenzonitrile (12.28 g) in CH₃CN (100 mL) was added Boc-piperazine (11.43 g) followed by DIPEA (16 mL). The resulting mixture was heated at 80° C. for 48 h and then concentrated in vacuo. The residue was dissolved in toluene and filtered. The filtrate was concentrated in vacuo and chromatographed on SiO₂ (Hexanes to 35% EtOAc/Hexanes) to yield the title compound as a white solid.

MS (electrospray): exact mass for C₁₆H₂₀BrN₃O₂, 365.07; found m/z 266.1, 268.1 [M-Boc]⁺, 312.1, 314.1 [M-^{t}Bu]⁺, 390.1, 392.1 [M+Na]⁺.

### Step B: 4-(2-Bromo-4-carboxy-phenyl)-piperazine-1-carboxylic acid tert-butyl ester

4-(2-Bromo-4-carboxy-phenyl)piperazine-1-carboxylic acid tert-butyl ester was prepared according to the procedure as described in Example 15, Step B reacting 5 g of the product prepared as in Step A above to yield the title compound as a colorless foam.

MS (electrospray): exact mass for C₁₆H₂₁BrN₂O₄, 384.07; found m/z 331.1, 333.1 [M-^{t}Bu]⁺, 385.1, 387.1 [M+H]⁺, 407.1, 409.1 [M+Na]⁺.

### Step C: 1,1-Dimethylethyl 4-{4-[({[(1Z)-1-aminoethylidene]amino}oxy)carbonyl]-2-bromophenyl}piperazine-1-carboxylate

1,1-Dimethylethyl 4-{4-[({[(1Z)-1-aminoethylidene]amino}oxy)carbonyl]-2-bromophenyl}piperazine-1-carboxylate was prepared according to the procedure as described in Example 15, Step C reacting 5.04 g of the product prepared as in Step B above to yield the title compound.

MS (electrospray): exact mass for C₁₈H₂₅BrN₄O₄, 440.11; found m/z 385.1, 387.1 [M-^{t}Bu]⁺, 441.2, 443.2 [M+H]⁺.

### Step D: 4-[2-Bromo-4-(3-methyl-[1,2,4]oxadiazol-5-yl)-phenyl]-piperazine-1-carboxylic acid tert-butyl ester

To a solution of the product of Step C (3.93 g) in ^{t}BuOH (90 mL) was added NaOAc (804 mg) in H₂O (0.6 mL). The resulting mixture was heated at 85° C. for 23 h and concentrated in vacuo. The residue was dissolved in toluene and filtered. The filtrate was concentrated in vacuo and chromatographed on SiO₂ (5% EtOAc/Hexanes to 50% EtOAc/Hexanes) to yield the title compound.

MS (electrospray): exact mass for C₁₈H₂₃BrN₄O₃, 422.10; found m/z 367.1, 369.1 [M-^{t}Bu]⁺, 423.2, 425.2 [M+H]⁺, 445.1, 447.1 [M+Na]⁺.

### Step E: 2-{4-[2-Bromo-4-(3-methyl-[1,2,4]oxadiazol-5-yl)-phenyl]-piperazin-1-yl}-N,N-diethyl-2-phenyl-acetamide

2-{4-[2-Bromo-4-(3-methyl-[1,2,4]oxadiazol-5-yl)-phenyl]-piperazin-1-yl}-N,N-diethyl-2-phenyl-acetamide was prepared according to the procedure as described in Example 27, Step B reacting 530 mg of the product prepared as in Step D above to yield the title compound.

MS (electrospray): exact mass for C₂₅H₃₀BrN₅O₂, 511.16; found m/z 512.2, 514.2 [M+H]⁺. ¹H NMR (500 MHz, CDCl₃): 8.28 (d, J=2.02 Hz, 1H), 7.97 (dd, J=8.43, 2.03 Hz, 1H), 7.49-7.44 (m, 2H), 7.40-7.30 (m, 3H), 7.09-7.06 (d, J=8.43 Hz, 1H), 4.28 (s, 1H), 3.53-3.36 (m, 2H), 3.33-3.13 (m, 6H), 2.79-2.66 (m, 4H), 2.44 (s, 3H), 1.13-1.03 (m, 6H).

### Example 56

### N,N-Diethyl-2-[4-(3-fluoro-4′-methyl-biphenyl-4-yl)-piperazin-1-yl]-2-phenyl-acetamide (compound #204)

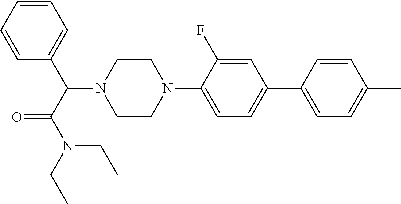

The title compound was prepared according to the procedure as described in Example 44 above, with the appropriate substitutions.

MS (ESI): mass calcd. for C₂₉H₃₄FN₃O, 459.61; m/z found, 460.3 [M+H]⁺

¹H NMR (CDCl₃): 7.48-7.45 (m, 2H), 7.43-7.39 (m, 2H), 7.39-7.29 (m, 3H), 7.28-7.17 (m, 4H), 6.99-6.93 (m, 1H), 4.23 (s, 1H), 3.51-3.37 (m, 2H), 3.33-3.11 (m, 6H), 2.75-2.67 (m, 4H), 2.37 (s, 3H), 1.12-1.03 (m, 6H)

### Example 57

### N,N-Diethyl-2-[4-(3-fluoro-4′-methoxy-biphenyl-4-yl)-piperazin-1-yl]-2-phenyl-acetamide (Compound #205)

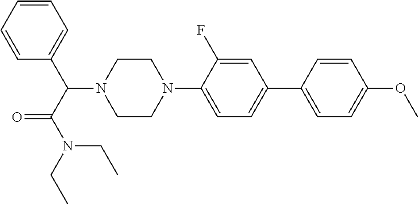

The title compound was prepared according to the procedure as described in Example 44 above, with the appropriate substitutions.

MS (ESI): mass calcd. for C₂₉H₃₄FN₃O₂, 475.61; m/z found, 476.3 [M+H]⁺

¹H NMR (CDCl₃) 7.49-7.43 (m, 2H), 7.39-7.29 (m, 3H), 7.25-7.17 (m, 2H), 6.99-6.92 (m, 3H), 4.24 (s, 1H), 3.83 (s, 3H), 3.52-3.37 (m, 2H), 3.33-3.11 (m, 6H), 2.77-2.65 (m, 4H), 1.12-1.02 (m, 6H)

### Example 58

### N,N-Diethyl-2-[4-(3-fluoro-3′-methyl-biphenyl-4-yl))-piperazin-1-yl]-2-phenyl-acetamide (Compound #206)

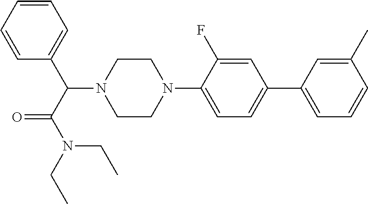

The title compound was prepared according to the procedure as described in Example 44 above, with the appropriate substitutions.

MS (ESI): mass calcd. for C₂₉H₃₄FN₃O, 459.61; m/z found, 460.3 [M+H]⁺

¹H NMR (CDCl₃): 7.49-7.45 (m, 2H), 7.39-7.22 (m, 8H), 7.14-7.10 (m, 1H), 6.99-6.94 (m, 1H), 4.23 (s, 1H), 3.52-3.37 (m, 2H), 3.33-3.12 (m, 6H), 2.77-2.66 (m, 4H), 1.12-1.03 (m, 6H)

### Example 59

### 2-[4-(3,4′-Difluoro-biphenyl-4-yl)-piperazin-1-yl]-N,N-diethyl-2-phenyl-acetamide (Compound #207)

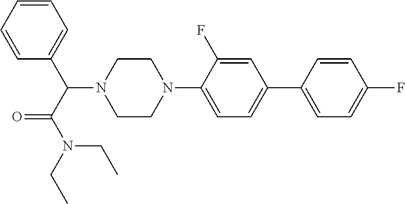

The title compound was prepared according to the procedure as described in Example 44 above, with the appropriate substitutions.

MS (ESI): mass calcd. for C₂₈H₃₁F₂N₃O, 463.57; m/z found, 464.3 [M+H]⁺

¹H NMR (CDCl₃): 7.50-7.44 (m, 4H), 7.39-7.29 (m, 3H), 7.25-7.16 (m, 2H), 7.12-7.06 (m, 2H), 7.00-6.93 (m, 1H), 4.25 (5, 1H), 3.52-3.36 (m, 2H), 3.33-3.12 (m, 6H), 2.78-2.65 (m, 4H), 1.12-1.02 (m, 6H)

### Example 60

### N,N-Diethyl-2-{4-[2-fluoro-4-(5-methyl-2H-[1,2,4]triazol-3-yl)-phenyl]-piperazin-1-yl}-2-phenyl-acetamide (compound #208)

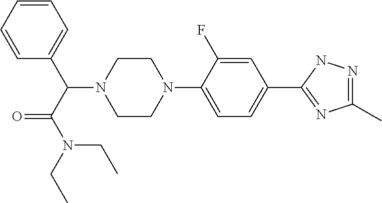

### Step A: N,N-Diethyl-2-{4-[2-fluoro-4-(5-methyl-2H-[1,2,4]triazol-3-yl)-phenyl]-piperazin-1-yl}-2-phenyl-acetamide

To a heterogeneous mixture of NaOMe (127 mg) in EtOH (5 mL) was added acetamidine hydrochloride. After 40 min the product prepared as in Example 23, Step C (500 mg) was added, and the resulting mixture heated at 100° C. for 48 h. The resulting mixture was concentrated in vacuo and the residue chromatographed on SiO₂ (CH₂Cl₂ to 8% 2 M NH₃ in MeOH/CH₂Cl₂) to yield the title compound as a white foam.

MS (electrospray): exact mass for C₂₅H₃₁FN₆O, 450.25; found m/z 451.3 [M+H]⁺. ¹H NMR (600 MHz, CDCl₃): 12.53-12.18 (br s, 1H), 7.73-7.63 (m, 2H), 7.49-7.44 (m, 2H), 7.39-7.30 (m, 3H), 6.92-6.86 (m, 1H), 4.22 (s, 1H), 3.52-3.42 (m, 2H), 3.35-3.27 (m, 1H), 3.27-3.10 (m, 5H), 2.74-2.61 (m, 4H), 2.54 (s, 3H), 1.12-1.05 (m, 6H).

### Example 61

### 2-(2-Bromo-5-fluoro-phenyl)-N,N-diethyl-2-{4-[2-fluoro-4-(3-methyl-[1,2,4]oxadiazol-5-yl)-phenyl]-piperazin-1-yl}-acetamide (Compound #209)

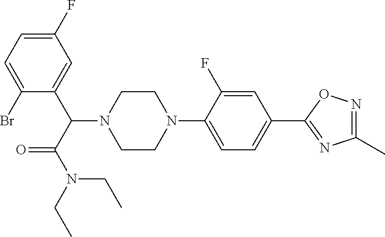

The title compound was prepared according to the process described in Example 40 reacting 1-[2-fluoro-4-(3-methyl-[1,2,4]oxadiazol-5-yl)-phenyl]piperazine 2HCl (200 mg, 0.60 mmol), 2-bromo-2-(2-bromo-5-fluoro-phenyl)-N,N-diethyl-acetamide (288 mg, 0.78 mmol), TEA (207 mg, 2.0 mmol) and CH₃CN (10 ml) and purifying the isolated residue by chromatography (SiO₂, O-4% acetone/DCM, gradient) to yield the title compound.

MS (ESI) mass calcd. for C₂₅H₂₈F₂N₅O₂Br, 548.42; m/z found, 548.2 [M+H]⁺

¹H NMR (CDCl₃): 7.81-7.75 (m, 1H), 7.74-7.67 (m, 1H), 7.58-7.51 (m, 2H), 7.98-7.89 (m, 2H), 4.79 (s, 1H), 3.50-3.35 (m, 2H), 3.34-3.19 (m, 6H), 2.89-2.80 (m, 2H), 2.77-2.67 (m, 2H), 2.43 (s, 3H), 1.15-1.05 (m, 6H).

### Example 62

### N,N-Diethyl-2-{4-[2-fluoro-4-(3-methyl-[1,2,4]oxadiazol-5-yl)-phenyl]-piperazin-1-yl}-2-(4-fluoro-phenyl)-acetamide (Compound #210)

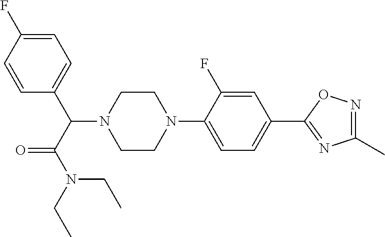

The title compound was prepared according to the procedure describe din Example 61, by substituting the appropriate reagents and purifying the isolated residue by chromatography (SiO₂, O-4% acetone/DCM, gradient) to yield title compound.

MS (ESI) mass calcd. for C₂₅H₂₉F₂N₅O₂, 469.54; m/z found, 470.3 [M+H]⁺

¹H NMR (CDCl₃): 7.80-7.76 (dd, J=8.4, 1.8, 1H), 7.73-7.67 (dd, J=13.4, 1.8, 1H), 7.47-7.41 (m, 2H), 7.09-7.02 (m, 2H), 6.94 (t, J=8.6, 1H), 4.25 (s, 1H), 3.50-3.33 (m, 2H), 3.32-3.18 (m, 6H), 2.74-2.62 (m, 4H), 2.43 (s, 3H), 1.14-1.04 (m, 6H).

### Example 63

### N,N-Diethyl-2-{4-[2-fluoro-4-(3-methyl-[1,2,4]oxadiazol-5-yl)-phenyl]-piperazin-1-yl}-2-(3-fluoro-phenyl)-acetamide (Compound #211)

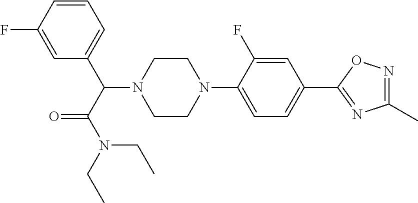

The title compound was prepared according to the procedure describe din Example 61, by substituting the appropriate reagents and purifying the isolated residue by chromatography (SiO₂, O-5% acetone/DCM, gradient) to yield title compound.

MS (ESI) mass calcd. for C₂₅H₂₉F₂N₅O₂, 469.54; m/z found, 470.3 [M+H]⁺

¹H NMR (CDCl₃): 7.85-7.79 (m, 1H), 7.77-7.70 (m, 1H), 7.40-7.21 (m, 3H), 7.10-6.94 (m, 2H), 4.32 (s, 1H), 3.54-3.39 (m, 2H), 3.38-3.22 (m, 6H), 2.83-2.66 (m, 4H), 2.46 (s, 3H), 1.20-1.07 (m, 6H).

### Example 64

### N,N-Diethyl-2-{4-[2-fluoro-4-(5-isopropyl-2H-[1,2,4]triazol-3-yl)-phenyl]-piperazin-1-yl}-2-phenyl-acetamide (Compound #212)

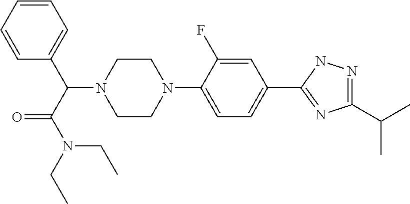

N,N-Diethyl-2-{4-[2-fluoro-4-(5-isopropyl-2H-[1,2,4]triazol-3-yl)-phenyl]-piperazin-1-yl}-2-phenyl-acetamide was prepared according to the procedure as described in Example 60 reacting the product prepared as in Example 23, Step C (500 mg) and isobutyramidine hydrochloride (215 mg) to yield the title compound.

MS (electrospray): exact mass for C₂₇H₃₅FN₆O, 478.29; found m/z 479.3

[M+H]⁺

¹H NMR (500 MHz, CDCl₃): 11.74-11.37 (br s, 1H), 7.75-7.65 (m, 2H), 7.49-7.43 (m, 2H), 7.38-7.29 (m, 3H), 6.95-6.88 (m, 1H), 4.24 (s, 1H), 3.51-3.40 (m, 2H), 3.35-3.11 (m, 7H), 2.74-2.63 (m, 4H), 1.40 (d, J=6.99 Hz, 6H), 1.13-1.03 (m, 6H).

### Example 65

### {4-[2-Fluoro-4-(3-isopropyl-[1,2,4]oxadiazol-5-yl)-phenyl]-piperazin-1-yl}-phenyl-acetic acid methyl ester (Compound #213)

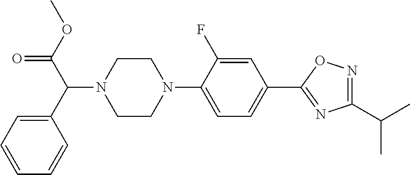

The title compound was prepared according to the process outlined in Example 6 above, with the appropriate substituent changes.

MS (ESI): mass calcd. for C₂₄H₂₇FN₄O₃, 438.2; m/z found, 439.3 [M+H]⁺

¹H NMR (CDCl₃): 7.84-7.73 (m, 2H), 7.48-7.32 (m, 5H), 6.97 (t, J=8.6, 1H), 4.11 (s, 1H), 3.72 (s, 3H), 3.35-3.27 (m, 4H), 3.18-3.10 (m, 1H), 2.72-2.66 (m, 4H), 1.43-1.37 (m, 6H).

### Example 66

### N,N-Diethyl-2-[4-(3-fluoro-2′-methoxy-biphenyl-4-yl)-piperazin-1-yl]-2-phenyl-acetamide (Compound #214)

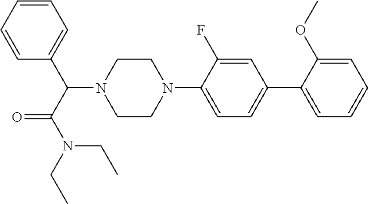

The title compound was prepared according to the procedure as described in Example 44 above, with the appropriate substitutions.

MS (ESI): mass calcd. for C₂₉H₃₄FN₃O₂, 475.61; m/z found, 476.3 [M+H]⁺

¹H NMR (CDCl₃): 7.49-7.45 (m, 2H), 7.39-7.20 (m, 7H), 7.06-6.91 (m, 3H), 4.24 (s, 1H), 3.81 (s, 3H), 3.51-3.37 (m, 2H), 3.33-3.12 (m, 6H), 2.76-2.65 (m, 4H), 1.11-1.03 (m, 6H)

### Example 67

### 2-{4-[4-(1,5-Dimethyl-1H-[1,2,4]triazol-3-yl)-2-fluoro-phenyl]-piperazin-1-yl}-N,N-diethyl-2-phenyl-acetamide (Compound #215)

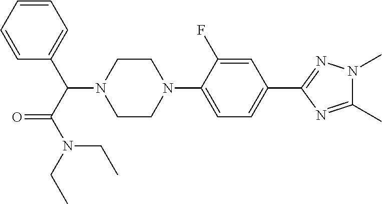

To a homogeneous solution of the product prepared as in Example 60 in DMF (3 mL) was added NaH (12 mg). After 30 min CH₃I (0.024 mL) was added. The resulting mixture was aged for several hours and the quenched by the addition of H₂O. The aqueous layer was extracted with Et₂O and the combined organic extracts were dried over Na₂SO₄, filtered and concentrated in vacuo to yield a residue. The residue was chromatographed on SiO₂ (CH₂Cl₂ to 8% 2M NH₃ in MeOH/CH₂Cl₂) to yield the title compound.

MS (electrospray): exact mass for C₂₆H₃₃FN₆O, 464.27; found m/z 465.3 [M+H]⁺

¹H NMR (500 MHz, CDCl₃): 7.73-7.64 (m, 2H), 7.50-7.43 (m, 2H), 7.39-7.29 (m, 3H), 6.95-6.90 (m, 1H), 4.26 (s, 1H), 3.81 (s, 3H), 3.49-3.36 (m, 2H), 3.34-3.12 (m, 6H), 2.79-2.62 (m, 4H), 2.46 (s, 3H), 1.13-1.01 (m, 6H).

### Example 68

### N,N-Diethyl-2-{4-[2-fluoro-4-(5-isopropyl-1-methyl-1H-[1,2,4]triazol-3-yl)-phenyl]-piperazin-1-yl}-2-phenyl-acetamide (Compound #216)

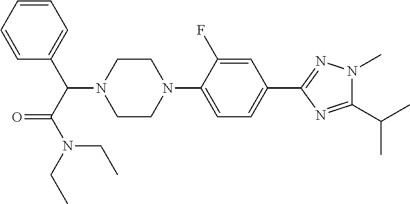

To a solution of the product prepared as in Example 64 (411 mg) in THF (6 mL) was added NaO^{t}Bu (125 mg). After 10 min CH₃I (0.059 mL) was added and the resulting mixture aged for 12 h. The resulting mixture was diluted with H₂O and CH₂Cl₂. The layers were separated and the aqueous layer extracted with CH₂Cl₂. The combined organic layers were dried over Na₂SO₄, filtered and concentrated in vacuo to yield a yellow solid. he solid was chromatographed on SiO₂ (EtOAc) to yield the title compound as a white solid.

MS (electrospray): exact mass for C₂₈H₃₇FN₆O, 492.30; found m/z 493.4 [M+H]⁺

¹H NMR (500 MHz, CDCl₃): 7.75-7.67 (m, 2H), 7.49-7.43 (m, 2H), 7.38-7.28 (m, 3H), 6.96-6.89 (m, 1H), 4.25 (s, 1H), 3.83 (s, 3H), 3.50-3.38 (m, 2H), 3.34-3.12 (m, 6H), 3.12-3.02 (m, 1H), 2.77-2.62 (m, 4H), 1.37 (d, J=6.89 Hz, 6H), 1.12-1.01 (m, 6H).

### Example 69

### 2-{4-[2-Cyano-4-(3-methyl-[1,2,4]oxadiazol-5-yl)-phenyl]-piperazin-1-yl}-N,N-diethyl-2-phenyl-acetamide (Compound #217)

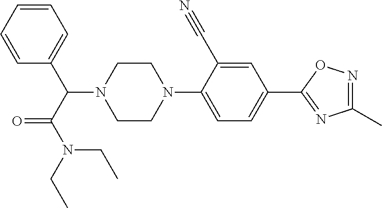

### Step A: 3-Cyano-4-fluoro-benzoic acid

To a solution of 2-fluoro-5-formyl-benzonitrile (2.57 g) in ^{t}BuOH (110 mL) was added 2-methyl-2-butene (86 mL) followed by a mixture of NaClO₂ (14 g) and NaH₂PO₄ (14 g) in H₂O (140 mL) dropwise via an addition funnel at 0° C. The resulting mixture was allowed to warm to room temperature and the volatiles removed in vacuo. The remaining residue was diluted with H₂O and washed 3× with hexanes. The aqueous layer was acidified to pH 3 with 4 N HCl and extracted with CH₂Cl₂ and EtOAc. The combined organic layers were washed with brine, dried over Na₂SO₄, filtered and concentrated in vacuo to yield the title compound as a white solid.

MS (electrospray): exact mass for C₈H₄FNO₂, 165.02; found m/z 166.1 [M+H]⁺.

### Step B: (1E)-N′-{[(3-Cyano-4-fluorophenyl)carbonyl]oxy}ethanimidamide

(1E)-N′-{[(3-Cyano-4-fluorophenyl)carbonyl]oxy}ethanimidamide was prepared according to the procedure as described in Example 15, Step C (except triethylamine was not added during the acid chloride formation) reacting 1.6 g of the product prepared as in STEP A above and 1.85 mL of DIPEA (in place of Et₃N during the acylation step) to yield the title compound.

MS (electrospray): exact mass for C₁₀H₈FN₃O₂, 221.06; found m/z 222.2 [M+H]⁺.

### Step C: 2-Fluoro-5-(3-methyl-[1,2,4]oxadiazol-5-yl)-benzonitrile

2-Fluoro-5-(3-methyl-[1,2,4]oxadiazol-5-yl)-benzonitrile was prepared according to the procedure as described in Example 55, Step D reacting 1.84 g of the product prepared as in STEP B above, to yield the title compound as a white solid.

MS (electrospray): exact mass for C₁₀H₆FN₃O, 203.05; found m/z 204.1 [M+H]⁺.

### Step D: 4-[2-Cyano-4-(3-methyl-[1,2,4]oxadiazol-5-yl)-phenyl]-piperazine-1-carboxylic acid tert-butyl ester

4-[2-Cyano-4-(3-methyl-[1,2,4]oxadiazol-5-yl)-phenyl]-piperazine-1-carboxylic acid tert-butyl ester was prepared according to Example 55, Step A reacting 1.35 g of the product prepared as in STEP C above, to yield the title compound as a white solid.

MS (electrospray): exact mass for C₁₉H₂₃N₅O₃, 369.18; found m/z 270.2 [M-Boc]⁺, 314.2 [M-^{t}Bu]⁺, 392.2 [M+Na]⁺.

### Step E″ 2-{4-[2-Cyano-4-(3-methyl-[1,2,4]oxadiazol-5-yl)-phenyl]-piperazin-1-yl}-N,N-diethyl-2-phenyl-acetamide

2-{4-[2-Cyano-4-(3-methyl-[1,2,4]oxadiazol-5-yl)-phenyl]-piperazin-1-yl}-N,N-diethyl-2-phenyl-acetamide was prepared according to the procedure as described in Example 26, Step B reacting 302 mg of the product prepared as in STEP D above, to yield the title compound.

MS (electrospray): exact mass for C₂₆H₃₀N₆O₂, 458.24; found m/z 459.3 [M+H]⁺

¹H NMR (500 MHz, CDCl₃): 8.26 (d, J=2.13 Hz, 1H), 8.11 (dd, J=8.85, 2.16 Hz, 1H), 7.47-7.41 (m, 2H), 7.40-7.31 (m, 3H), 7.01 (d, J=8.89 Hz, 1H), 4.28 (s, 1H), 3.52-3.24 (m, 7H), 3.23-3.13 (m, 1H), 2.81-2.69 (m, 4H), 2.45 (s, 3H), 1.12-1.02 (m, 6H).

### Example 70

### 2-{4-[2-Cyano-4-(3-isopropyl-[1,2,4]oxadiazol-5-yl)-phenyl]-piperazin-1-yl}-N,N-diethyl-2-phenyl-acetamide (Compound #218)

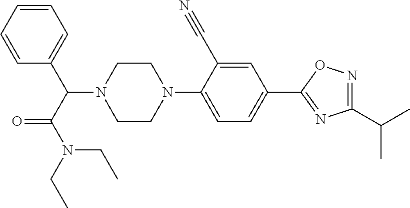

2-{4-[2-Cyano-4-(3-isopropyl-[1,2,4]oxadiazol-5-yl)-phenyl]-piperazin-1-yl}-N,N-diethyl-2-phenyl-acetamide was prepared according to the procedure as described in Example 69 reacting the product prepared as in Example 69, STEP A (1.04 g) and N-hydroxy-isobutyramidine (772 mg) to yield the title compound.

MS (electrospray): exact mass for C₂₈H₃₄N₆O₂, 486.27; found m/z 487.3 [M+H]⁺

¹H NMR (500 MHz, CDCl₃): 8.28 (d, J=2.12 Hz, 1H), 8.12 (dd, J=8.83, 2.15 Hz, 1H), 7.47-7.43 (m, 2H), 7.39-7.30 (m, 3H), 7.01 (d, J=8.88 Hz, 1H), 4.27 (s, 1H), 3.52-3.08 (m, 9H), 2.80-2.70 (m, 4H), 1.40-1.37 (m, 6H), 1.12-1.02 (m, 6H).

### Example 71

### {4-[2-Fluoro-4-(3-methyl-[1,2,4]oxadiazol-5-yl)-phenyl]-piperazin-1-yl}-phenyl-acetic acid tert-butyl ester (Compound #219)

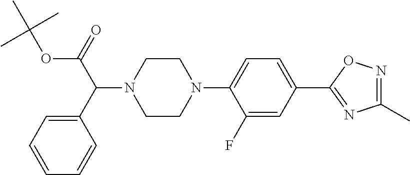

### Step A: Bromo-phenyl-acetic acid tert-butyl ester

To a solution of bromo-phenyl-acetic acid (1.7 g) in ^{t}BuOH (40 mL) was added DMAP (290 mg) and Boc₂O (3.45 g) in two portions. After 6 h, the resulting mixture was concentrated in vacuo and the residue chromatographed on SiO₂ (Hexanes to 10% EtOAc/Hexanes) to yield the title compound as a colorless oil.

MS (electrospray): exact mass for C₁₂H₁₅BrO₂, 270.03; found m/z 293.1, 295.1 [M+Na]⁺.

### Step B: {4-[2-Fluoro-4-(3-methyl-[1,2,4]oxadiazol-5-yl)-phenyl]-piperazin-1-yl}-phenyl-acetic acid tert-butyl ester

{4-[2-Fluoro-4-(3-methyl-[1,2,4]oxadiazol-5-yl)-phenyl]-piperazin-1-yl}-phenyl-acetic acid tert-butyl ester was prepared according to the procedure of Example 27, Step B reacting 997 mg of the product prepared as in STEP A above and 1.03 g of the product prepared as in Example 15, Step E to yield the title compound as a pale yellow solid.

MS (electrospray): exact mass for C₂₅H₂₉FN₄O₃, 452.22; found m/z 453.3 [M+H]⁺

¹H NMR (600 MHz, CDCl₃): 7.80 (dd, J=8.47, 1.81 Hz, 1H), 7.72 (dd, J=13.41, 1.96 Hz, 1H), 7.47-7.42 (m, 2H), 7.38-7.30 (m, 3H), 6.99-6.93 (m, 1H), 3.96 (s, 1H), 3.31-3.23 (m, 4H), 2.70-2.63 (m, 4H), 2.44 (s, 3H), 1.40 (s, 9H).

### Example 72

### N,N-Diethyl-2-{4-[2-fluoro-4-(5-isopropyl-2-methyl-2H-[1,2,4]triazol-3-yl)-phenyl]-piperazin-1-yl}-2-phenyl-acetamide (Compound #220)

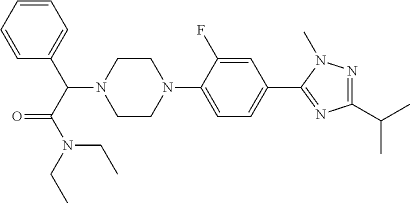

N,N-Diethyl-2-{4-[2-fluoro-4-(5-isopropyl-2-methyl-2H-[1,2,4]triazol-3-yl)-phenyl]-piperazin-1-yl}-2-phenyl-acetamide was prepared according to the procedure as described in Example 69 reacting the product prepared as in Example 64 (41 mg) to yield the title compound as a colorless oil.

MS (electrospray): exact mass for C₂₈H₃₇FN₆O, 492.30; found m/z 493.4 [M+H]⁺

¹H NMR (500 MHz, CDCl₃): 7.48-7.43 (m, 2H), 7.40-7.30 (m, 5H), 7.01-6.95 (m, 1H), 4.26 (s, 1H), 3.90 (s, 3H), 3.52-3.36 (m, 2H), 3.34-3.13 (m, 6H), 3.06 (sept., J=6.96 Hz, 1H), 2.79-2.63 (m, 4H), 1.35 (d, J=6.96 Hz, 6H), 1.12-1.02 (m, 6H).

### Example 73

### 2-[4-(Diethylcarbamoyl-phenyl-methyl)-piperazin-1-yl]-5-(3-isopropyl-[1,2,4]oxadiazol-5-yl)-benzamide (compound #221)

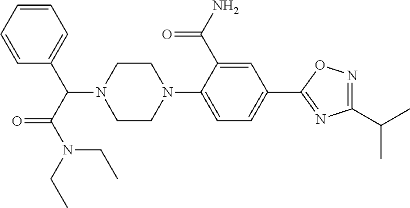

### Step A: 5-(3-Isopropyl-[1,2,4]oxadiazol-5-yl)-2-piperazin-1-yl-benzonitrile

5-(3-Isopropyl-[1,2,4]oxadiazol-5-yl)-2-piperazin-1-yl-benzonitrile was prepared according to the procedure as described in Example 15, Step E reacting the product prepared as in Example 70, Step D (1.35 g) to yield the title compound.

MS (electrospray): exact mass for C₁₆H₁₉N₅O, 297.16; found m/z 298.3 [M+H]⁺

### Step B: 5-(3-Isopropyl-[1,2,4]oxadiazol-5-yl)-2-piperazin-1-yl-benzamide

To a heterogeneous mixture of KOH (85 mg) in ^{t}BuOH (2 mL) was added the product of Step A (100 mg). The resulting mixture was heated at 100° C. for 40 min. The resulting mixture was concentrated in vacuo, dissolved in toluene and filtered. The filtrate was concentrated in vacuo to yield the title compound as a pale yellow solid.

MS (electrospray): exact mass for C₁₆H₂₁N₅O₂, 315.17; found m/z 316.3 [M+H]⁺.

### Step C: 2-[4-(Diethylcarbamoyl-phenyl-methyl)-piperazin-1-yl]-5-(3-isopropyl-[1,2,4]oxadiazol-5-yl)-benzamide

2-[4-(Diethylcarbamoyl-phenyl-methyl)-piperazin-1-yl]-5-(3-isopropyl-[1,2,4]oxadiazol-5-yl)-benzamide was prepared according to the procedure as described in Example 15, Step F reacting 89 mg of the product prepared as in Step B above to yield the title compound as a white solid.

MS (electrospray): exact mass for C₂₈H₃₆N₆O₃, 504.28; found m/z 505.4 [M+H]⁺

¹H NMR (500 MHz, CDCl₃): 8.80 (d, J=2.14 Hz, 1H), 8.76-8.71 (br s, 1H), 8.15 (dd, J=8.46, 2.16 Hz, 1H), 7.46-7.31 (m, 5H), 7.25 (s, 1H), 5.93-5.88 (br s, 1H), 4.42 (s, 1H), 3.58-3.49 (m, 1H), 3.38-3.20 (m, 2H), 3.19-3.05 (m, 6H), 2.94-2.85 (m, 2H), 2.80-2.71 (m, 2H), 1.38 (d, J=6.94 Hz, 6H), 1.12 (t, J=7.07 Hz, 3H), 1.04 (t, J=7.07 Hz, 3H).

### Example 74

### 2-{4-[2-Cyano-4-(3-methyl-[1,2,4]oxadiazol-5-yl)-phenyl]-piperazin-1-yl}-N,N-diethyl-2-(4-methoxy-phenyl)-acetamide (Compound #250)

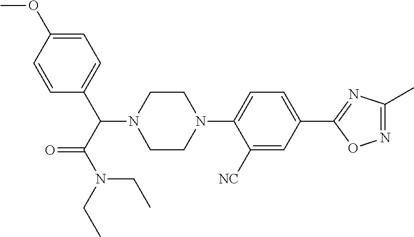

The title compound was prepared according to the process as described in Example 69 above, with the appropriate substitutions.

MS (ESI): mass calcd. for C₂₇H₃₂N₆O₃, 488.59; m/z found, 489.3 [M+H]⁺

¹H NMR (CDCl₃): 8.26-8.24 (m, 1H), 8.13-8.08 (m, 1H), 7.38-7.34 (m, 2H), 7.03-6.99 (m, 1H), 6.91-6.87 (m, 2H), 4.22 (s, 1H), 3.81 (s, 3H), 3.51-3.39 (m, 5H), 3.39-3.23 (m, 2H), 3.22-3.13 (m, 1H), 2.79-2.68 (m, 4H), 2.44 (s, 3H), 1.12-1.03 (m, 6H)

### Example 75

### 2-{4-[2-Cyano-4-(3-methyl-[1,2,4]oxadiazol-5-yl)-phenyl]-piperazin-1-yl}-N,N-diethyl-2-(2-methoxy-phenyl)-acetamide (Compound #251)

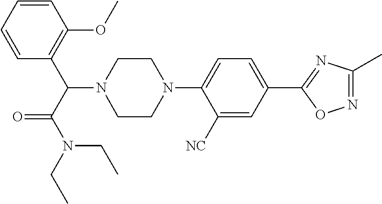

The title compound was prepared according to the process as described in Example 69 above, with the appropriate substitutions.

MS (ESI): mass calcd. for C₂₇H₃₂N₆O₃, 488.59; m/z found, 489.3 [M+H]⁺

¹H NMR (CDCl₃): 8.26-8.24 (m, 1H), 8.12-8.08 (m, 1H), 7.56-7.53 (m, 1H), 7.32-7.27 (m, 1H), 7.03-6.91 (m, 3H), 4.88 (s, 1H), 3.89 (s, 3H), 3.50-3.38 (m, 5H), 3.34-3.23 (m, 2H), 3.16-3.08 (m, 1H), 2.79-2.72 (m, 4H), 2.44 (s, 3H), 1.07 (t, J=7.1 Hz, 3H), 0.95 (t, J=7.1 Hz, 3H)

### Example 76

### 2-{4-[2-Cyano-4-(3-methyl-[1,2,4]oxadiazol-5-yl)-phenyl]-piperazin-1-yl}-N,N-diethyl-2-thiophen-2-yl-acetamide (Compound #252)

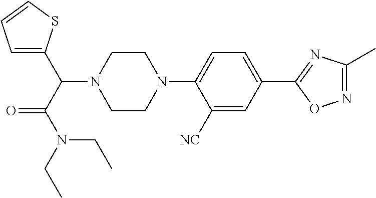

The title compound was prepared according to the process as described in Example 69 above, with the appropriate substitutions.

MS (ESI): mass calcd. for C₂₄H₂₈N₆O₂S, 464.59; m/z found, 465.3 [M+H]⁺

¹H NMR (CDCl₃): 8.26-8.25 (m, 1H), 8.12-8.09 (m, 1H), 7.35-7.32 (m, 1H), 7.04-6.97 (m, 3H), 4.69 (s, 1H), 3.50-3.32 (m, 8H), 2.92-2.85 (m, 2H), 2.77-2.70 (m, 2H), 2.45 (m, 3H), 1.19-1.11 (m, 6H)

### Example 77

### 2-[4-(2′-Cyano-3-fluoro-biphenyl-4-yl)-piperazin-1-yl]-N,N-diethyl-2-phenyl-acetamide (Compound #253)

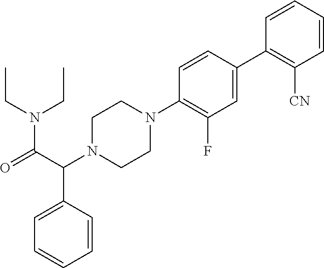

The title compound was prepared according to the process as described in Example 44 above, with the appropriate substitutions.

MS (ESI): mass calcd. for C₂₉H₃₁FN₄O, 470.59; m/z found, 471.3 [M+H]⁺

¹H NMR (CDCl₃): 7.84-7.81 (m, 0.5H), 7.74-7.71 (m, 1H), 7.62-7.58 (m, 1H), 7.49-7.42 (m, 3.5H), 7.41-7.30 (m, 3H), 7.29-7.25 (m, 1H), 7.24-7.18 (m, 1H), 7.02-6.97 (m, 1H), 4.27 (5, 1H), 3.50-3.38 (m, 2H), 3.33-3.17 (m, 6H), 2.77-2.67 (m. 4H), 1.12-1.03 (m, 6H)

### Example 78

### N,N-Diethyl-2-[4-(3-fluoro-2′-methyl-biphenyl-4-yl)-piperazin-1-yl]-2-phenyl-acetamide (Compound #254)

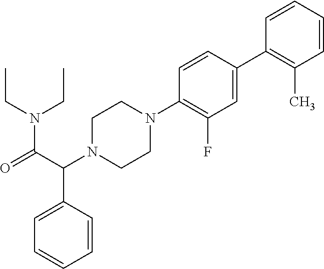

The title compound was prepared according to the process as described in Example 44 above, with the appropriate substitutions.

MS (ESI): mass calcd. for C₂₉H₃₄FN₃O, 459.61; m/z found, 460.3 [M+H]⁺

¹H NMR (CDCl₃): 7.49-7.45 (m, 2H), 7.39-7.29 (m, 3H), 7.25-7.16 (m, 4H), 7.01-6.91 (m, 3H), 4.26 (s, 1H), 3.51-3.38 (m, 2H), 3.33-3.14 (m, 6H), 2.77-2.67 (m, 4H), 2.27 (s, 3H), 1.12-1.03 (m, 6H)

### Example 79

### 2-[4-(3-Cyano-2′-methoxy-biphenyl-4-yl)-piperazin-1-yl]-N,N-diethyl-2-phenyl-acetamide (Compound #255)

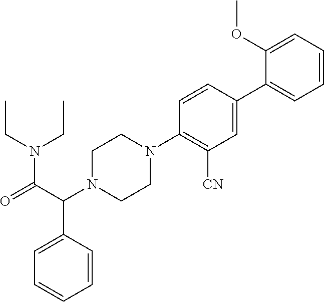

### Step A: 4-(4-Bromo-2-cyano-phenyl)-piperazine-1-carboxylic acid tert-butyl ester

A mixture of 5-bromo-2-fluoro-benzonitrile (1.05 g), piperazine-1-carboxylic acid tert-butyl ester (1.08 g) and Na₂CO₃ (1.03 g) in DMF was heated to 100° C. for 24 h. The resulting mixture was cooled, diluted with water (50 mL) and extracted with diethyl ether. The resulting mixture was dried over MgSO₄, the solvent was removed and the resulting residue purified on SiO₂ (hexanes/EtOAc 0 to 25%) to yield the title compound.

### Step B: 2-[4-(4-Bromo-2-cyano-phenyl)-piperazin-1-yl]-N,N-diethyl-2-phenyl-acetamide

A solution of 4-(4-Bromo-2-cyano-phenyl)piperazine-1-carboxylic acid tert-butyl ester (151.6 mg) prepared as in STEP A above was dissolved in CH₂Cl₂ (3 mL) and the resulting mixture treated with TFA (1 mL). The resulting mixture was stirred for 6 h, then the solvent was removed to yield a residue. To the residue was added 2-bromo-N,N-diethyl-2-phenyl-acetamide (0.14 g), Na₂CO₃ (0.20 g) and DMF (3 mL). The resulting mixture was stirred for 16 h, then diluted with water (50 mL) and extracted with diethyl ether. The resulting mixture was dried over MgSO₄, the solvent was removed and the resulting residue purified on SiO₂ (hexanes/EtOAc 0 to 50%) to yield the title compound.

### Step C: 2-[4-(3-Cyano-2′-methoxy-biphenyl-4-yl)-piperazin-1-yl]-N,N-diethyl-2-phenyl-acetamide

The title compound was prepared according to the process as described in Example 44, Step C above, reacting the compound as prepared in STEP B above, with the appropriate substitutions.

MS (ESI): mass calcd. for C₃₀H₃₄N₄O₂, 482.63; m/z found, 483.3 [M+H]⁺

¹H NMR (CDCl₃): 7.74-7.72 (m, 1H), 7.63-7.61 (m, 1H), 7.48-7.45 (m, 2H), 7.38-7.29 (m, 4H), 7.26-7.23 (m, 1H), 7.03-6.95 (m, 3H), 4.24 (s, 1H), 3.81 (s, 3H), 3.49-3.36 (m, 2H), 3.34-3.16 (m, 6H), 2.76-2.68 (m, 4H), 1.11-1.03 (m, 6H)

### Example 80

### N,N-Diethyl-2-{4-[2-formyl-4-(3-isopropyl-[1,2,4]oxadiazol-5-yl)-phenyl]-piperazin-1-yl}-2-phenyl-acetamide (Compound #256)

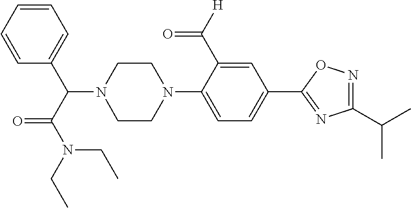

### Step A: 4-[2-Carbamoyl-4-(3-isopropyl-[1,2,4]oxadiazol-5-yl)-phenyl]-piperazine-1-carboxylic acid tert-butyl

To a heterogeneous mixture of KOH (562 mg) in ^{t}BuOH (11 mL) was added the compound prepared as in Example 70, STEP D above (884 mg). The resulting mixture was heated at reflux for 50 min and then concentrated in vacuo. The residue was partitioned between H₂O and EtOAc. The combined organic layers were dried over Na₂SO₄, filtered and concentrated in vacuo to yield a yellow solid. Chromatography on SiO₂ (Hexanes to 100% EtOAc/Hexanes) yielded the title compound as a pale yellow solid.

MS (electrospray): exact mass for C₂₁H₂₉N₅O₄, 415.22; found m/z 416.3 [M+H]⁺.

### Step B: 4-[2-Hydroxymethyl-4-(3-isopropyl-[1,2,4]oxadiazol-5-yl)-phenyl]-piperazine-1-carboxylic acid tert-butyl ester

To a heterogeneous mixture of the product prepared as in Step A above (724 mg) in CH₃CN (17 mL) was added DMAP (21 mg) followed by Boc₂O (797 mg). The resulting mixture was stirred for 14 h and then concentrated in vacuo. Chromatography on SiO₂ (Hexanes to 35% EtOAc/Hexanes) yielded an intermediate that was dissolved in EtOH and treated with NaBH₄ (230 mg). The resulting mixture was stirred for 4 d and then treated with acetone (2 mL). After 2 h the resulting mixture was concentrated in vacuo and the residue was partitioned between H₂O and EtOAc. The aqueous layer was extracted with EtOAc and CH₂Cl₂, and the combined organic extracts were dried over Na₂SO₄, filtered and concentrated in vacuo. Chromatography on SiO₂ (Hexanes to 45% EtOAc/Hexanes) yielded the title compound as a white solid.

MS (electrospray): exact mass for C₂₁H₃₀N₄O₄, 402.23; found m/z 403.3 [M+H]⁺.

### Step C: 4-[2-Formyl-4-(3-isopropyl-[1,2,4]oxadiazol-5-yl)-phenyl]-piperazine-1-carboxylic acid tert-butyl ester

To a solution of the product prepared as in STEP B above (522 mg) in CH₂Cl₂ (12 mL) was added Dess-Martin periodinane (715 mg) at 0° C. The resulting mixture was allowed to warm to room temperature and diluted with Et₂O, then treated with saturated aqueous NaHCO₃ solution and Na₂S₂O₃. The combined organic layers were dried over Na₂SO₄, filtered and concentrated in vacuo to yield a pale yellow solid. Chromatography on SiO₂ (Hexanes to 40 EtOAc/Hexanes) yielded the title compound as a pale yellow solid.

MS (electrospray): exact mass for C₂₁H₂₈N₄O₄, 400.21; found m/z 401.3 [M+H]⁺.

### Step D: N,N-Diethyl-2-{4-[2-formyl-4-(3-isopropyl-[1,2,4]oxadiazol-5-yl)-phenyl]-piperazin-1-yl}-2-phenyl-acetamide

N,N-Diethyl-2-{4-[2-formyl-4-(3-isopropyl-[1,2,4]oxadiazol-5-yl)-phenyl]-piperazin-1-yl}-2-phenyl-acetamide was prepared according to the procedure as described in Example 27, Step B reacting the product prepared as in STEP C above to yield the title compound.

MS (electrospray): exact mass for C₂₈H₃₅N₅O₃, 489.27; found m/z 490.4 [M+H]⁺

¹H NMR (500 MHz, CDCl₃): 10.17 (s, 1H), 8.49 (d, J=2.19 Hz, 1H), 8.17 (dd, J=8.63, 2.19 Hz, 1H), 7.47-7.42 (m, 2H), 7.40-7.30 (m, 3H), 7.12 (d, J=8.63 Hz, 1H), 4.34 (s, 1H), 3.54-3.44 (m, 1H), 3.43-3.09 (m, 8H), 2.88-2.70 (m, 4H), 1.38 (d, J=6.95 Hz, 6H), 1.14-1.01 (m, 6H).

### Example 81

### N,N-Diethyl-2-{4-[2-hydroxymethyl-4-(3-isopropyl-[1,2,4]oxadiazol-5-yl)-phenyl]-piperazin-1-yl}-2-phenyl-acetamide (Compound #257)

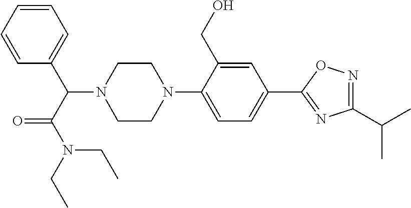

To a solution of the product prepared as in Example 80 above (105 mg) in MeOH (2 mL) was added NaBH₄ (12 mg). After 3 h the reaction was quenched by the addition of acetone and concentrated in vacuo. The residue was partitioned between saturated aqueous NaHCO₃ solution and CH₂Cl₂. The combined organic extracts were dried over Na₂SO₄, filtered and concentrated in vacuo to yield an oil. Chromatography on SiO₂ (5% EtOAc/Hexanes to 80 EtOAc/Hexanes) yielded the title compound as a white solid.

MS (electrospray): exact mass for C₂₈H₃₇N₅O₃, 491.29; found m/z 492.4 [M+H]⁺

¹H NMR (500 MHz, CDCl₃): 8.02-7.98 (m, 2H), 7.47-7.42 (m, 2H), 7.40-7.30 (m, 3H), 7.25-7.21 (m, 1H), 4.80 (s, 2H), 4.32-4.24 (m, 2H), 3.52-3.34 (m, 2H), 3.32-3.01 (m, 7H), 2.80-2.65 (m, 4H), 1.38 (d, J=6.95 Hz, 6H), 1.12-1.02 (m, 6H).

### Example 82

### N,N-Diethyl-2-{4-[4-(3-isopropyl-[1,2,4]oxadiazol-5-yl)-2-methylaminomethyl-phenyl]-piperazin-1-yl}-2-phenyl-acetamide (Compound #258)

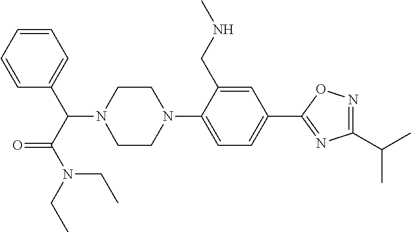

To a solution of the product prepared as in Example 80 above (284 mg) in MeOH (5 mL) was added CH₃NH₂, 40% wt in H₂O (0.506 mL). The resulting mixture was stirred at room temperature for 5 h and then NaBH₄ (44 mg) was added. The resulting mixture was stirred for 14 h at room temperature and then concentrated in vacuo. The residue was partitioned between saturated aqueous NaHCO₃ solution and CH₂Cl₂. The combined organic extracts were dried over Na₂SO₄, filtered and concentrated in vacuo to yield a viscous orange oil. Chromatography on SiO₂ (CH₂Cl₂ to 8% 2 M NH₃ in MeOH/CH₂Cl₂) yielded the title compound as a colorless foam.

MS (electrospray): exact mass for C₂₉H₄₀N₆O₂, 504.32; found m/z 505.4 [M+H]⁺

¹H NMR (500 MHz, CDCl₃): 8.07 (d, J=2.06 Hz, 1H), 7.95 (dd, J=8.38, 2.06 Hz, 1H), 7.49-7.44 (m, 2H), 7.39-7.29 (m, 3H), 7.13 (d, J=8.38 Hz, 1H), 4.33 (s, 1H), 3.80 (s, 2H), 3.53-3.37 (m, 2H), 3.32-3.01 (m, 7H), 2.81-2.65 (m, 4H), 2.43 (s, 3H), 1.38 (d, J=6.95 Hz, 6H), 1.13-1.03 (m, 6H).

### Example 83

### 2-{4-[2-Dimethylaminomethyl-4-(3-isopropyl-[1,2,4]oxadiazol-5-yl)-phenyl]-piperazin-1-yl}-N,N-diethyl-2-phenyl-acetamide (Compound #259)

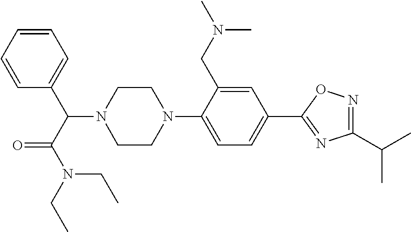

To a solution of the product prepared as in Example 82 above (83 mg) in MeOH (3 mL) was added CH₂O, 37% wt in H₂O followed by Na(OAc)₃BH (70 mg). After 4 h at room temperature the resulting mixture was concentrated in vacuo and partitioned between CH₂Cl₂ and 1 N NaOH. The combined organic extracts were dried over Na₂SO₄, filtered and concentrated in vacuo to yield a pale yellow oil. Chromatography on SiO₂ (CH₂Cl₂ to 5% 2 M NH₃ in MeOH/CH₂Cl₂) yielded a colorless oil which was dissolved in Et₂O and treated with excess 1 N HCl in Et₂O. After 20 min the resulting mixture was concentrated in vacuo to yield the title compound as its corresponding dihydrochloride salt.

MS (electrospray): exact mass for C₃₀H₄₂N₆O₂, 518.34; found m/z 519.4 [M+H]⁺

¹H NMR (500 MHz, MeOH-d₄): 8.35-8.21 (m, 2H), 7.80-7.70 (m, 2H), 7.62-7.51 (m, 4H), 6.02-5.81 (br s, 1H), 4.60-4.44 (m, 2H), 4.10-3.73 (m, 3H), 3.56-3.06 (m, 9H), 2.97 (s, 6H), 2.86-2.73 (m, 1H), 1.37 (d, J=6.95 Hz, 6H), 1.12 (t, J=7.08 Hz, 3H), 0.92 (t, J=7.08 Hz, 3H).

### Example 84

### N,N-Diethyl-2-{4-[4-(3-isopropyl-[1,2,4]oxadiazol-5-yl)-2-methoxymethyl-phenyl]-piperazin-1-yl}-2-phenyl-acetamide (Compound #260)

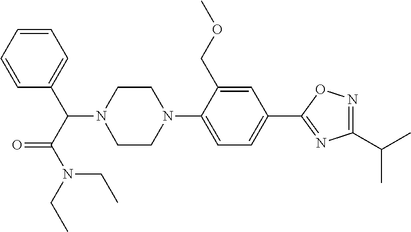

To a solution of the product prepared as in Example 81 above (75 mg) in THF (5 mL) was added NaH (4 mg) followed 10 min later by MeI (0.011 mL) at 0° C. After allowing the mixture to warm to room temperature, the reaction mixture was again cooled to 0° C. and an additional portion of NaH (15 mg) and CH₃I (0.011 mL) was added. The resulting mixture was allowed to warm to room temperature and diluted with H₂O. The aqueous layer was extracted with CH₂Cl₂ and the combined organic layers were dried over Na₂SO₄, filtered and concentrated in vacuo to yield a viscous yellow oil. Chromatography SiO₂ (5% EtOAc/Hexanes to 60 EtOAc/Hexanes) yielded the title compound as a pale yellow foam.

MS (electrospray): exact mass for C₂₉H₃₉N₅O₃, 505.31; found m/z 506.3 [M+H]⁺

¹H NMR (500 MHz, CDCl₃): 8.16 (d, J=2.11 Hz, 1H), 7.97 (dd, J=8.43, 2.11 Hz, 1H), 7.49-7.44 (m, 2H), 7.40-7.30 (m, 3H), 7.09 (d, J=8.43 Hz, 1H), 4.48 (s, 2H), 4.41 (s, 1H), 3.55-3.33 (m, 5H), 3.32-3.01 (m, 7H), 2.87-2.66 (m, 4H), 1.38 (d, J=6.95 Hz, 6H), 1.11 (t, J=7.08 Hz, 3H), 1.05 (t, J=7.08 Hz, 3H).

### Example 85

### 2-{4-[2-Cyano-4-(3-methyl-[1,2,4]oxadiazol-5-yl)-phenyl]-piperazin-1-yl}-N-ethyl-2-phenyl-acetamide (Compound #261)

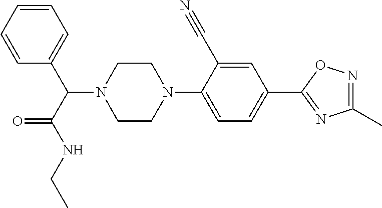

2-{4-[2-Cyano-4-(3-methyl-[1,2,4]oxadiazol-5-yl)-phenyl]-piperazin-1-yl}-N-ethyl-2-phenyl-acetamide was prepared according to the procedure described in Example 55, Step A reacting N-ethyl-2-phenyl-2-piperazin-1-yl-acetamide (106 mg) and the product prepared as in Example 69, Step C above (87 mg) to yield the title compound as a white solid.

MS (electrospray): exact mass for C₂₄H₂₆N₆O₂, 430.21; found m/z 431.3 [M+H]⁺

¹H NMR (600 MHz, CDCl₃): 8.28 (d, J=2.11 Hz, 1H), 8.14 (dd, J=8.83, 2.11 Hz, 1H), 7.39-7.30 (m, 5H), 7.04 (d, J=8.83 Hz, 1H), 6.94 (t, J=5.67 Hz, 1H), 3.91 (s, 1H), 3.51-3.41 (m, 4H), 3.39-3.33 (m, 2H), 2.71-2.61 (m, 4H), 2.45 (s, 3H), 1.18 (t, J=7.25 Hz, 3H).

### Example 86

### 2-{4-[2-Cyano-4-(3-methyl-[1,2,4]oxadiazol-5-yl)-phenyl]-piperazin-1-yl}-N,N-diethyl-2-(4-fluoro-phenyl)-acetamide (Compound #262)

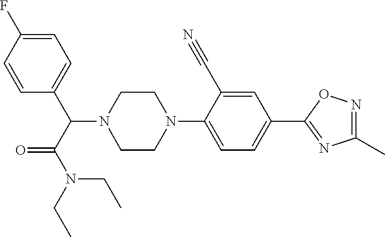

### Step A: 5-(3-Methyl-[1,2,4]oxadiazol-5-yl)-2-piperazin-1-yl-benzonitrile

5-(3-Methyl-[1,2,4]oxadiazol-5-yl)-2-piperazin-1-yl-benzonitrile was prepared according to the procedure described in Example 15, STEP E reacting the product prepared as in Example 69, STEP D above (1.7 g) to yield a pale yellow solid.

MS (electrospray): exact mass for C₁₄H₁₆N₆O, 269.13; found m/z 270.2 [M+H]⁺.

### Step B: 2-{4-[2-Cyano-4-(3-methyl-[1,2,4]oxadiazol-5-yl)-phenyl]-piperazin-1-yl}-N,N-diethyl-2-(4-fluoro-phenyl)-acetamide

2-{4-[2-Cyano-4-(3-methyl-[1,2,4]oxadiazol-5-yl)-phenyl]-piperazin-1-yl}-N,N-diethyl-2-(4-fluoro-phenyl)-acetamide was prepared according to the procedure described in Example 27, STEP B reacting the product prepared in STEP A above (245 mg) and 2-bromo-N,N-diethyl-2-(4-fluoro-phenyl)-acetamide (288 mg) to yield the title compound.

MS (electrospray): exact mass for C₂₆H₂₉FN₆O₂, 476.23; found m/z 477.3 [M+H]⁺

¹H NMR (500 MHz, CDCl₃): 8.26 (d, J=2.12 Hz, 1H), 8.11 (dd, J=8.84, 2.12 Hz, 1H), 7.48-7.41 (m, 2H), 7.09-6.99 (m, 3H), 4.27 (s, 1H), 3.50-3.17 (m, 8H), 2.78-2.68 (m, 4H), 2.44 (s, 3H), 1.13-1.06 (m, 6H).

### Example 87

### 2-{4-[2-Cyano-4-(3-methyl-[1,2,4]oxadiazol-5-yl)-phenyl]-piperazin-1-yl}-N,N-diethyl-2-(3-fluoro-phenyl)-acetamide (Compound #263)

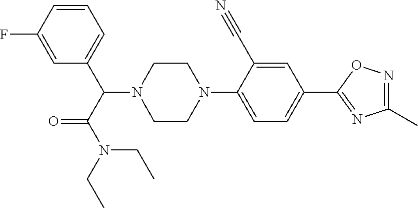

2-{4-[2-Cyano-4-(3-methyl-[1,2,4]oxadiazol-5-yl)-phenyl]-piperazin-1-yl}-N,N-diethyl-2-(3-fluoro-phenyl)-acetamide was prepared according to the procedure described in Example 86, STEP B reacting the product prepared as in Example 86, STEP A (32 mg) and 2-bromo-N,N-diethyl-2-(3-fluoro-phenyl)-acetamide (34 mg) to yield the title compound.

MS (electrospray): exact mass for C₂₆H₂₉FN₆O₂, 476.23; found m/z 477.4 [M+H]⁺

¹H NMR (500 MHz, CDCl₃): 8.26 (d, J=2.12 Hz, 1H), 8.12 (dd, J=8.83, 2.12 Hz, 1H), 7.36-7.29 (m, 1H), 7.28-7.19 (m, 2H), 7.06-6.98 (m, 2H), 4.30 (s, 1H), 3.50-3.18 (m, 8H), 2.81-2.70 (m, 4H), 2.44 (s, 3H), 1.13-1.06 (m, 6H).

### Example 88

### N,N-Diethyl-2-{4-[2-fluoro-4-(3-methyl-[1,2,4]thiadiazol-5-yl)-phenyl]-piperazin-1-yl}-2-phenyl-acetamide (Compound #271)

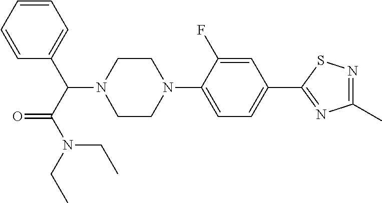

### Step A: 4-(4-Carbamoyl-2-fluoro-phenyl)-piperazine-1-carboxylic acid tert-butyl ester

A solution of the product of Example 1, Step A (2.15 g, 7.05 mmol) and KOH (1.78 g, 31.7 mmol) in t-BuOH (35 mL) was refluxed for 1 h. The solvent was removed and the residue partitioned between H₂O (20 mL) and EtOAc (20 mL). The aqueous layer was further extracted with EtOAc, and the combined organic layers were dried over Na₂SO₄, filtered and concentrated in vacuo to yield 4-(4-carbamoyl-2-fluoro-phenyl)-piperazine-1-carboxylic acid tert-butyl ester as a white solid.

MS (electrospray): exact mass for C₁₆H₂₂FN₃O₃, 323.16; found m/z 268.2

[M-tBu]⁺, 346.3 [M+Na]⁺.

### Step B: 4-(2-Fluoro-4-thiocarbamoyl-phenyl)-piperazine-1-carboxylic acid tert-butyl ester

To a solution of the product from Step A (1.90 g, 5.87 mmol) in THF (100 mL) was added Lawesson's reagent (1.19 g, 2.93 mmol). The mixture was stirred at ambient temperature for 48 h and then concentrated in vacuo to yield a residue, which was used in subsequent reactions without further purification.

### Step C: 4-[4-(1-Dimethylamino-ethylidenethiocarbamoyl)-2-fluoro-phenyl]-piperazine-1-carboxylic acid tert-butyl ester

To the residue prepared in Step B (250 mg, 0.736 mmol) was added dimethylacetamide dimethylacetal (5 mL). The resulting mixture was stirred at ambient temperature for 14 h and then concentrated in vacuo. The resulting residue was chromatographed on SiO₂ (Hexanes to 30% EtOAc/Hexanes) to yield 4-[4-(1-dimethylamino-ethylidenethiocarbamoyl)-2-fluoro-phenyl]-piperazine-1-carboxylic acid tert-butyl ester.

### Step D: 4-[2-Fluoro-4-(3-methyl-[1,2,4]thiadiazol-5-yl)-phenyl]-piperazine-1-carboxylic acid tert-butyl ester

To a solution of the product from Step C (125 mg, 0.305 mmol) in EtOH (2 mL) was added pyridine (0.05 mL, 0.61 mmol) followed by hydroxylamine-O-sulfonic acid (38 mg, 0.335 mmol) in MeOH (0.5 mL). The resulting mixture was stirred for 2 h at room temperature and then concentrated in vacuo. The resulting residue was chromatographed on SiO₂ (Hexanes to 35% EtOAc/Hexanes) to yield 4-[2-fluoro-4-(3-methyl-[1,2,4]thiadiazol-5-yl)-phenyl]-piperazine-1-carboxylic acid tert-butyl ester as a pale yellow solid.

MS (electrospray): exact mass for C₁₈H₂₃FN₄O₂S, 378.15; found m/z 323.2 [M-tBu]⁺, 379.2 [M+H]⁺.

### Step E: N,N-Diethyl-2-{4-[2-fluoro-4-(3-methyl-[1,2,4]thiadiazol-5-yl)-phenyl]-piperazin-1-yl}-2-phenyl-acetamide

N,N-Diethyl-2-{4-[2-fluoro-4-(3-methyl-[1,2,4]thiadiazol-5-yl)-phenyl]-piperazin-1-yl}-2-phenyl-acetamide was prepared according to the procedure as described in Example 1, Step D reacting the product prepared as in Step D above (62 mg) and 49 mg of 2-bromo-N,N-diethyl-2-phenylacetamide (49 mg) to yield N,N-diethyl-2-{4-[2-fluoro-4-(3-methyl-[1,2,4]thiadiazol-5-yl)-phenyl]-piperazin-1-yl}-2-phenyl-acetamide.

MS (electrospray): exact mass for C₂₅H₃₀FN₅OS, 467.22; found m/z 468.2 [M+H]⁺.

¹H NMR (600 MHz, CDCl₃): 7.63-7.57 (m, 2H), 7.47-7.43 (m, 2H), 7.39-7.30 (m, 3H), 6.95-6.90 (m, 1H), 4.26 (s, 1H), 3.51-3.36 (m, 2H), 3.31-3.14 (m, 6H), 2.77-2.72 (m, 2H), 2.71-2.65 (m, 5H), 1.09 (t, J=7.08 Hz, 3H), 1.04 (t, J=7.11 Hz, 3H).

### Example 89

### Neuropeptide Y2 Radioligand Binding Assay

KAN-Ts endogenously expressing Y2 receptors were used for the radioligand binding assay. Cells were grown to confluence on 150 cm² tissue culture plates, washed with phosphate-buffered saline (PBS), and scraped into 50 mL tubes. After centrifugation, the supernatant was aspirated, and the pellets frozen and stored at −80° C. Thawed pellets were homogenized with a polytron tissue grinder for 15 sec in 20 mM Tris-HCl, 5 mM EDTA. The homogenate was centrifuged at 800×g for 5 min and the collected supernatant was recentrifuged at 25000×g for 25 min. The resulting pellet was resuspended in binding buffer (20 mM HEPES, 120 mM NaCl, 0.22 mM KH₂PO₄, 1.3 mM CaCl₂, 0.8 mM MgSO₄). Membranes were incubated with [¹²⁵I]PYY (80 pM) in the presence or absence of test compound for 1 h at rt. The reaction was stopped by filtration through GF/C filter plates pre-soaked in 0.3% polyethylenimine and subsequently washed with Tris 50 mM, 5 mM EDTA buffer. Plates were dried for 1 h in a 55° C. oven, scintillation fluid was added and the radioactivity was counted in a Packard TopCount. Specific binding to the NPY receptor subtypes was determined by radioactivity that was bound in the presence of 1 mM NPY. Each binding experiment was repeated three to eight times, each in duplicate. IC₅₀ values (i.e. concentration of unlabelled peptide or antagonist required to compete for 50% of specific binding to the radioligand) were determined using the GraphPad Prism software (GraphPad Software Inc., San Diego Calif.) with a fit to a sigmoidal dose response curve. Apparent K values were as K=IC₅₀/(1+C/K_{D}), where C is concentration of the radioligand.

### Example 90

### Neuropeptide Y2 pK_{B} Assay

The assay was performed using the fluorimetric imaging plate reader (FLIPR) format as described in Dautzenberg, F. M., Biochemical Pharmacology 2005, 69, 1493.

KAN-Ts cells stably expressing chimeric G proteins were seeded at a density of 100,000 cells into poly-d-lysine coated 384-well blackwall, clear-bottom microtiter plates (Corning, N.Y.). One day later, the medium was removed and 50 μl loading medium DMEM high glucose, without serum, supplemented with 10 mM HEPES-acid, 0.1% BSA, 5 mM probenecid and 2 μM Fluo-3AM was added. Cells were loaded for 1 h at 37° C., washed twice with 50 μl assay buffer (5 mM HEPES-acid, 140 mM NaCl, 1 mM MgCl₂, 5 mM KCl, 10 mM glucose) and then 30 μl assay buffer was added. Cells were further pre-incubated at room temperature before adding agonists or agonists plus antagonists in 20 μl assay buffer and then measured on a T-channel fluorometric imaging plate reader (FLIPR, Molecular Devices, Sunnyvale, Calif.). Antagonistic potency values were converted to apparent pK_{B} values using a modified Cheng-Prusoff correction. Apparent pK_{B} was calculated as pK_{B}=−log IC₅₀/1+[conc agonist/EC₅₀].

Representative compounds of the present invention were tested for NPY Y₂ radioligand binding and pK_{B} activity, as described in Examples 89 and 90 above, with results as listed in Table 3 below.

TABLE 3
NPY Y2 Binding and pK_{B}

| Compound No. | IC₅₀ (μM) | pK_{B} |
|---|---|---|
| 3 | 0.24 | |
| 67 | 0.18 | 6.7 |
| 68 | 0.52 | |
| 69 | 0.13 | 7.3 |
| 70 | 0.48 | |
| 71 | 0.097 | |
| 75 | 0.6 | |
| 79 | 6.3 | |
| 80 | 0.041 | |
| 88 | 0.27 | |
| 89 | 0.85 | |
| 97 | 0.37 | |
| 105 | 0.16 | |
| 118 | 0.18 | |
| 119 | 0.085 | 7.05 |
| 124 | 1 | |
| 129 | 1.9 | |
| 130 | >10 | |
| 133 | 0.46 | |
| 144 | 1.1 | |
| 147 | 4.1 | |
| 149 | 0.99 | |
| 150 | 0.42 | |
| 151 | >10 | |
| 152 | 0.3 | |
| 154 | 0.22 | |
| 158 | 1.5 | |
| 176 | 1.9 | |
| 177 | 0.51 | |
| 178 | 10 | |
| 180 | 0.15 | |
| 181 | 0.53 | |
| 182 | 0.56 | |
| 183 | 3.1 | |
| 184 | 2.2 | |
| 185 | 0.12 | |
| 186 | 0.11 | |
| 187 | 5 | |
| 188 | 0.63 | |
| 189 | 0.48 | |
| 190 | 0.33 | |
| 191 | 0.34 | |
| 192 | 0.38 | |
| 193 | 0.42 | |
| 194 | 0.88- | |
| 195 | 0.9 | |
| 196 | 0.92 | |
| 197 | 0.075 | |
| 198 | 2.7 | |
| 199 | 2.8 | |
| 200 | 0.81 | |
| 201 | 0.74 | |
| 202 | 1 | |
| 203 | 0.15 | 6.7 |
| 204 | 0.77 | |
| 205 | 1.9 | |
| 206 | 0.48 | |
| 207 | 1.1 | |
| 208 | 2.9 | |
| 209 | 0.99 | |
| 210 | 0.29 | |
| 211 | 0.083 | |
| 212 | 0.83 | |
| 213 | 0.24 | |
| 214 | 0.078 | |
| 215 | 1.1 | |
| 216 | 1.95 | |
| 217 | 0.023 | 7.63 |
| 218 | 0.011 | 7.9 |
| 219 | 0.66 | |
| 220 | 1.0 | |
| 221 | 2.1 | |
| 226 | 1.0 | |
| 250 | 0.035 | |
| 251 | 0.49 | |
| 252 | 0.096 | |
| 253 | 8.3 | |
| 254 | 1.0 | |
| 255 | 0.03 | |
| 256 | 0.17 | |
| 257 | 0.34 | |
| 258 | 1.8 | |
| 259 | 2.4 | |
| 260 | 0.98 | |
| 261 | 0.41 | |
| 262 | 0.098 | |
| 263 | 0.035 | 7.7 |
| 270 | 0.062 | |
| 271 | 0.11 | |

### Example 91

### Prophetic Example, Pharmaceutical Composition

As a specific embodiment of an oral composition, 100 mg of the Compound #119 (prepared as in Example 15) or Compound #217 (prepared as in Example 69) is formulated with sufficient finely divided lactose to provide a total amount of 580 to 590 mg to fill a size 0 hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed:

1. A compound of formula (I) 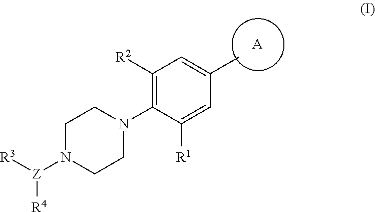
wherein
R¹ and R² are each independently selected from the group consisting of hydrogen, halogen, C₁₋₄alkyl, cyano, nitro, —NR^{A}R^{B}, —CH₂—NR^{A}R^{B}, —C(O)—NR^{A}R^{B} and —C(O)H; wherein R^{A} and R^{B} are each independently selected from the group consisting of hydrogen and C₁₋₄alkyl; provided that at least one or R¹ or R² is other than hydrogen; 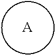
is selected from the group consisting of cycloalkyl, aryl, heteroaryl and heterocycloalkyl; wherein the cycloalkyl, aryl, heteroaryl or heterocycloalkyl is optionally substituted with one or more substituents independently selected from the group consisting of halogen, C₁₋₆alkyl, halogenated C₁₋₄alkyl, C₁₋₄alkoxy, —S—C₁₋₄alkyl, cyano, oxo, C₃₋₈cycloalkyl, phenyl, —C(O)OH, —C(O)—NR^{C}R^{D} and —C(NR^{C}R^{D})═N—OH; wherein R^{C} and R^{D} are each independently selected from the croup consisting of hydrogen and C₁₋₄alkyl;
provided that 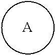
is other than 1-(pyrrolidin-2-one), 2-([1,2,5]-thiadiazolidine-1,1-dioxide), 2-(5-n-propyl-[1,2,5]-thiadiazolidine-1,1-dioxide), 2-(5-isopropyl-[1,2,5]-thiadiazolidine-1,1-dioxide), 2-(5-cyclopentyl-[1,2,5]-thiadiazolidine-1,1-dioxide) and 2-(5-(methoxycarbonyl)-[1,2,5]-thiadiazolidine-1,1-dioxide);
Z is selected from the group consisting of CH and CR⁰; wherein R⁰ is selected from the group consisting of —C₁₋₄alkyl;
R³ is selected from the group consisting of C₁₋₄alkyl, C₂₋₄alkenyl, cyano, O₃₋₈cycloalkyl, aryl, C₁₋₄aralkyl and 5 to 6 membered heteroaryl; wherein the aryl or heteroaryl, whether alone or as part of a substituent group is optionally substituted with one to two substituents independently selected from the group consisting of halogen, hydroxy, C₁₋₄alkyl, halogenated C₁₋₄alkyl, C₁₋₄alkoxy, halogenated C₁₋₄alkoxy, cyano, nitro, —NR^{E}R^{F} and —C(O)—NR^{E}R^{F}; wherein R^{E} and R^{F} are each independently selected from the group consisting of hydrogen and C₁₋₄alkyl;
R⁴ is selected from the group consisting of cyano, C₁₋₈alkyl, —C₁₋₄alkyl-OH, C₁₋₄alkyl-CN, —C(O)—NR^{G}R^{H}, —C(O)—C₁₋₄alkyl, —C(O)—O—C₁₋₄alkyl; wherein R^{G} and R^{H} are each independently selected from the group consisting of hydrogen and C₁₋₄alkyl; alternatively, R^{G} and R^{H} are taken together with the nitrogen atom to which they are bound to form a 4 to 8 membered saturated ring structure; wherein the 4 to 8 membered saturated ring structure is optionally substituted with one or more halogen;
or an enantiomers or pharmaceutically acceptable salt thereof.

2. A compound as in claim 1, wherein
R¹ and R² are each independently selected from the group consisting of hydrogen, halogen, C₁₋₄alkyl, —C₁₋₄alkyl-OH, —C₁₋₄alkyl-O—C₁₋₄alkyl, cyano, nitro, —NR^{A}R^{B}, —CH₂—NR^{A}R^{B}, —C(O)—NR^{A}R^{B} and —C(O)H; wherein R^{A} and R^{B} are each independently selected from the group consisting of hydrogen and C₁₋₄alkyl; provided that at least one or R¹ or R² is other than hydrogen; 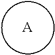
is selected from the group consisting of aryl, heteroaryl and heterocycloalkyl; wherein the aryl, heteroaryl or heterocycloalkyl is optionally substituted with one or more substituents independently selected from the group consisting of halogen, C₁₋₆alkyl, halogenated C₁₋₄alkyl, C₁₋₄alkoxy, —S—C₁₋₄alkyl, cyano, oxo, C₃₋₈cycloalkyl, phenyl, —C(O)O—C₁₋₄alkyl, —C(O)—NR^{C}R^{D} and —C(NR^{C}R^{D})═N—OH; wherein R^{C} and R^{D} are each independently selected from the group consisting of hydrogen and C₁₋₄alkyl;
provided that 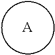
is other than 1-(pyrrolidin-2-one), 2-([1,2,5]-thiadiazolidine-1,1-dioxide), 2-(5-n-propyl-[1,2,5]-thiadiazolidine-1,1-dioxide), 2-(5-isopropyl-[1,2,5]-thiadiazolidine-1,1-dioxide), 2-(5-cyclopentyl-[1,2,5]-thiadiazolidine-1,1-dioxide) and 2-(5-(methoxycarbonyl)-[1,2,5]-thiadiazolidine-1,1-dioxide);
Z is selected from the group consisting of CH and CR⁰; wherein R⁰ is selected from the group consisting of —C₁₋₄alkyl;
R³ is selected from the group consisting of C₃₋₈cycloalkyl, aryl and 5 to 6 membered heteroaryl; wherein the aryl or heteroaryl, whether alone or as part of a substituent group is optionally substituted with one or more substituents independently selected from the group consisting of halogen, C₁₋₄alkyl, halogenated C₁₋₄alkyl, C₁₋₄alkoxy, halogenated C₁₋₄alkoxy, cyano, NR^{E}R^{F} and —C(O)—NR^{E}R^{F}; wherein R^{E} and R^{F} are each independently selected from the group consisting of hydrogen and C₁₋₄alkyl;
R⁴ is selected from the group consisting of cyano, C₁₋₆alkyl, —C(O)—NR^{G}R^{H}, —C(O)—C₁₋₄alkyl, —C(O)—O—C₁₋₄alkyl; wherein R^{G} and R^{H} are each independently selected from the group consisting of hydrogen and C₁₋₄alkyl; alternatively, R^{G} and R^{H} are taken together with the nitrogen atom to which they are bound to form a 4 to 8 membered saturated ring structure; wherein the 4 to 8 membered saturated ring structure is optionally substituted with one or more halogen;
or a pharmaceutically acceptable salt thereof.

3. A compound as in claim 2, wherein
R¹ is selected from the group consisting of halogen, cyano, C₁₋₄alkyl-OH, —CH₂—NR^{A}R^{B}, —C₁₋₂alkyl-O—C₁₋₂alkyl-, —C(O)H and —C(O)—NR^{A}R^{B}; wherein R^{A} and R^{B} are each independently selected from the group consisting of hydrogen and C₁₋₂alkyl; and R² is hydrogen; 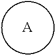
is selected from the group consisting of aryl, heteroaryl and heterocycloalkyl; wherein the aryl, heteroaryl or heterocycloalkyl is optionally substituted with one to two substituents independently selected from the group consisting of halogen, C₁₋₈alkyl, halogenated C₁₋₄alkyl, C₁₋₄alkoxy, cyano, oxo, —S—C₁₋₄alkyl, —C(O)OH, —C(O)O—C₁₋₄alkyl, —C(O)—NH₂, —C(NH₂)═N—OH, C₃₋₈cycloalkyl and phenyl;
provided that 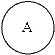
is other than 1-(pyrrolidin-2-one), 2-([1,2,5]-thiadiazolidine-1,1-dioxide), 2-(5-n-propyl-[1,2,5]-thiadiazolidine-1,1-dioxide), 2-(5-isopropyl-[1,2,5]-thiadiazolidine-1,1-dioxide), 2-(5-cyclopentyl-[1,2,5]-thiadiazolidine-1,1-dioxide) and 2-(5-(methoxycarbonyl)-[1,2,5]-thiadiazolidine-1,1-dioxide);
Z is selected from the group consisting of CH and CR⁰; wherein R⁰ is selected from the group consisting of C₁₋₂alkyl;
R³ is selected from the group consisting of aryl and 5 to 6 membered heteroaryl; wherein the aryl is optionally substituted with one to two substituents independently selected from the group consisting of halogen and C₁₋₄alkoxy;
R⁴ is selected from the group consisting of —C(O)—NR^{G}R^{H}, —C(O)—C₁₋₄alkyl and —C₁₋₄alkyl-O—C₁₋₄alkyl; wherein R^{G} and R^{H} are each independently selected from the group consisting of hydrogen and C₁₋₄alkyl; alternatively, alternatively, R^{G} and R^{H} are taken together with the nitrogen atom to which they are bound to form a 4 to 8 membered saturated ring structure; wherein the 4 to 8 membered saturated ring structure is optionally substituted with one to two halogen;
or a pharmaceutically acceptable salt thereof.

4. A compound as in claim 3, wherein
R¹ is selected from the group consisting of fluoro, bromo, cyano, hydroxy-methyl-, methylamino-methyl-, dimethylamino-methyl-, methoxy-methyl-, —C(O)H and —C(O)—NH₂; and R² is hydrogen; 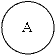
is selected from the group consisting of phenyl, 4-fluorophenyl, 3-chlorophenyl, 4-chlorophenyl, 2-methylphenyl, 3-methylphenyl, 4-methylphenyl, 2-methoxyphenyl, 4-methoxyphenyl, 2-cyanophenyl, 4-(1-methyl-pyrazolyl), 4-(3-methyl-pyrazolyl), 5-(1-phenyl-3-methyl-pyrazolyl), 3-(1-isopropyl-5-oxo-4,5-dihydro-pyrazolyl), 2-pyrrolyl, 2-(1-t-butoxycarbonyl-pyrrolyl), 2-pyridyl, 3-pyridyl, 2-oxazolyl, 2-(4,5-dihydro-4-methoxycarbonyl-oxazolyl), 2-(4-methoxycarbonyl-oxazolyl), 2-(5-ethyl-oxazolyl), 2-(5-methyl-[1,3,4]-oxadiazolyl), 2-(5-ethyl-[1,3,4]-oxadiazolyl), 2-(5-isopropyl-[1,3,4]-oxadiazolyl), 3-[1,2,4]-oxadiazolyl, 3-(5-methyl-[1,2,4]-oxadiazolyl), 3-(5-n-butyl-[1,2,4]-oxadiazolyl), 3-(5-isopropyl-[1,2,4]-oxadiazolyl), 3-(5-t-butyl-[1,2,4]-oxadiazolyl), 3-(5-(3-n-pentyl)[1,2,4]-oxadiazolyl), 3-(5-fluoromethyl-[1,2,4]-oxadiazolyl), 3-(5-trifluoromethyl-[1,2,4]-oxadiazolyl), 3-(5-cyano-[1,2,4]-oxadiazolyl), 3-(5-methylthio-[1,2,4]-oxadiazolyl), 3-(5-carboxy-[1,2,4]-oxadiazolyl), 3-(5-ethoxycarbonyl-[1,2,4]-oxadiazolyl), 3-(5-(C(NH₂)═N(OH))-[1,2,4]-oxadiazolyl), 3-(5-(aminocarbonyl)[1,2,4]-oxadiazolyl), 3-(4-methyl-5-oxo-[1,2,4]-oxadiazolyl), 3-(5-cyclopropyl-[1,2,4]-oxadiazolyl), 3-(5-cyclobutyl-[1,2,4]-oxadiazolyl), 3-(5-(1-(1-(S)methyl-n-propyl)-[1,2,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 3-(1,5-dimethyl-[1,2,4]-triazolyl), 3-(1-methyl-5-isopropyl-[1,2,4]-triazolyl), 5-(3-isopropyl-[1,2,4]-triazolyl, 5-(1-methyl-3-isopropyl-[1,2,4]-triazolyl) and 5-(3-methyl-[1,2,4]-thiadiazoyl);
Z is selected from the group consisting of CH and C(CH₃);
R³ is selected from the group consisting of phenyl, 3-fluoro-phenyl, 4-fluoro-phenyl, 2-bromo-5-fluoro-phenyl, 2-methoxy-phenyl, 4-methoxy-phenyl and 2-thienyl;
R⁴ is selected from the group consisting of ethylamino-carbonyl-, diethylamino-carbonyl-, N-methyl-N-ethyl-amino-carbonyl, 1-pyrrolidinyl-carbonyl-, 1-(4,4-difluoro-piperidinyl)-carbonyl-, methyl-carbonyl-, methoxy-carbonyl-, ethoxy-carbonyl- and t-butoxy-carbonyl-;
or a pharmaceutically acceptable salt thereof.

5. A compound as in claim 4, wherein
R¹ is selected from the group consisting of fluoro, bromo, cyano, hydroxy-methyl-, methoxy-methyl- and —C(O)H; and R² is hydrogen; 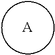
is selected from the group consisting of phenyl, 3-chlorophenyl, 4-chlorophenyl, 3-methylphenyl, 4-methylphenyl, 2-methoxyphenyl, 4-methoxyphenyl, 4-(1-methyl-pyrazolyl), 4-(3-methyl-pyrazolyl), 5-(1-phenyl-3-methyl-pyrazolyl), 3-(1-isopropyl-5-oxo-4,5-dihydro-pyrazolyl), 2-pyrrolyl, 2-pyridyl, 2-(5-ethyl-oxazolyl), 2-(5-methyl-[1,3,4]-oxadiazolyl), 2-(5-ethyl-[1,3,4]-oxadiazolyl), 2-(5-isopropyl-[1,3,4]-oxadiazolyl), 3-(5-methyl-[1,2,4]-oxadiazolyl), 3-(5-n-butyl-[1,2,4]-oxadiazolyl), 3-(5-isopropyl-[1,2,4]-oxadiazolyl), 3-(5-t-butyl-[1,2,4]-oxadiazolyl), 3-(5-(3-n-pentyl)[1,2,4]-oxadiazolyl), 3-(5-fluoromethyl-[1,2,4]-oxadiazolyl), 3-(5-trifluoromethyl-[1,2,4]-oxadiazolyl), 3-(5-methylthio-[1,2,4]-oxadiazolyl), 3-(5-cyclopropyl-[1,2,4]-oxadiazolyl), 3-(5-cyclobutyl-[1,2,4]-oxadiazolyl), 3-(5-(1-(1-(S)methyl-n-propyl)[1,2,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl), 5-(3-isopropyl-[1,2,4]-triazolyl and 5-(3-methyl-[1,2,4]-thiadiazolyl);
Z is CH;
R³ is selected from the group consisting of phenyl, 3-fluoro-phenyl, 4-fluoro-phenyl, 2-bromo-5-fluoro-phenyl, 2-methoxy-phenyl, 4-methoxy-phenyl and 2-thienyl;
R⁴ is selected from the group consisting of ethylamino-carbonyl-, diethylamino-carbonyl-, N-methyl-N-ethyl-amino-carbonyl, 1-pyrrolidinyl-carbonyl-, 1-(4,4-difluoro-piperidinyl)-carbonyl-, methoxy-carbonyl-, ethoxy-carbonyl- and t-butoxy-carbonyl-;
or a pharmaceutically acceptable salt thereof.

6. A compound as in claim 5, wherein
R¹ is selected from the group consisting of fluoro, bromo, cyano, hydroxy-methyl- and —C(O)H; and R² is hydrogen; 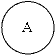
is selected from the group consisting of phenyl, 3-methylphenyl, 2-methoxyphenyl, 5-(1-phenyl-3-methyl-pyrazolyl), 3-(1-isopropyl-5-oxo-4,5-dihydro-pyrazolyl), 2-(5-ethyl-oxazolyl), 2-(5-methyl-[1,3,4]-oxadiazolyl), 2-(5-ethyl-[1,3,4]-oxadiazolyl), 2-(5-isopropyl-[1,3,4]-oxadiazolyl), 3-(5-methyl-[1,2,4]-oxadiazolyl), 3-(5-isopropyl-[1,2,4]-oxadiazolyl), 3-(5-(3-n-pentyl)[1,2,4]-oxadiazolyl), 3-(5-fluoromethyl-[1,2,4]-oxadiazolyl), 3-(5-trifluoromethyl-[1,2,4]-oxadiazolyl), 3-(5-cyclopropyl-[1,2,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl and 5-(3-methyl-[1,4]-thidiazolyl);
Z is CH;
R³ is selected from the group consisting of phenyl, 3-fluoro-phenyl, 4-fluoro-phenyl, 2-methoxy-phenyl, 4-methoxy-phenyl and 2-thienyl;
R⁴ is selected from the group consisting of ethylamino-carbonyl-, diethylamino-carbonyl-, N-methyl-N-ethyl-amino-carbonyl, 1-pyrrolidinyl-carbonyl-, 1-(4,4-difluoro-piperidinyl)-carbonyl-, methoxy-carbonyl- and ethoxy-carbonyl-;
or a pharmaceutically acceptable salt thereof.

7. A compound as in claim 6, wherein
R¹ is selected from the group consisting of fluoro, bromo, cyano and —C(O)H; and R² is hydrogen; 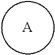
is selected from the group consisting of 2-methoxyphenyl, 5-(1-phenyl-3-methyl-pyrazolyl), 3-(5-methyl-[1,2,4]-oxadiazolyl), 3-(5-isopropyl-[1,2,4]-oxadiazolyl), 3-(5-fluoromethyl-[1,2,4]-oxadiazolyl), 3-(5-trifluoromethyl-[1,2,4]-oxadiazolyl), 5-(3-methyl-[1,2,4]-oxadiazolyl), 5-(3-ethyl-[1,2,4]-oxadiazolyl), 5-(3-isopropyl-[1,2,4]-oxadiazolyl and 5-(3-methyl-[1,2,4]-thiadiazolyl);
Z is CH;
R³ is selected from the group consisting of phenyl, 3-fluoro-phenyl, 4-fluoro-phenyl, 4-methoxy-phenyl and 2-thienyl;
R⁴ is selected from the group consisting of ethylamino-carbonyl-, diethylamino-carbonyl-, N-methyl-N-ethyl-amino-carbonyl and 1-pyrrolidinyl-carbonyl-;
or a pharmaceutically acceptable salt thereof.

8. A compound as in claim 7, wherein
R¹ is selected from the group consisting of fluoro and cyano; and R² is hydrogen; 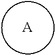
is selected from the group consisting of 2-methoxyphenyl, 5-(3-methyl-[1,2,4]-oxadiazolyl), 5-(3-ethyl-[1,2,4]-oxadiazolyl) and 5-(3-isopropyl-[1,2,4]-oxadiazolyl;
Z is CH;
R³ is selected from the group consisting of phenyl, 3-fluoro-phenyl, 4-fluoro-phenyl, 4-methoxy-phenyl and 2-thienyl;
R⁴ is selected from the group consisting of ethylamino-carbonyl-, diethylamino-carbonyl- and N-methyl-N-ethyl-amino-carbonyl-;
or a pharmaceutically acceptable salt thereof.

9. A compound as in claim 4, wherein
R¹ is selected from the group consisting of fluoro, bromo and cyano; and R² is hydrogen; 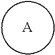
is selected from the group consisting of 3-(5-methyl-[1,2,4]-oxadiazolyl), 3-(5-isopropyl-[1,2,4]-oxadiazolyl), 5-(3-methyl-[1,2,4]-oxadiazolyl), 5-(3-ethyl-[1,2,4]-oxadiazolyl) and 5-(3-isopropyl-[1,2,4]-oxadiazolyl);
Z is CH;
R³ is selected from the group consisting of phenyl and 3-fluorophenyl;
R⁴ is diethylaminocarbonyl;
or a pharmaceutically acceptable salt thereof.

10. A pharmaceutical composition comprising a pharmaceutically acceptable carrier and a compound of claim 1.

11. A pharmaceutical composition made by mixing a compound of claim 1 and a pharmaceutically acceptable carrier.

12. A process for making a pharmaceutical composition comprising mixing a compound of claim 1 and a pharmaceutically acceptable carrier.

13. A method of treating a disorder mediated by the NPY Y2 receptor, comprising administering to a subject in need thereof, a therapeutically effective amount of a compound as in claim 1.

14. A method as in claim 13, wherein the disorder mediated by the NPY Y2 receptor is selected from the group consisting of anxiolytic disorders, depression; pain, injured mammalian nerve tissue; conditions responsive to treatment with a neurotrophic factor; neurological disorders; bone loss; cardiovascular diseases; sleep-wake state disorders, substance abuse and addiction related disorders; obesity; obesity-related disorders, disorders responsive to modulation of endocrine function, inovulation and infertility.

15. A method as in claim 13, wherein the disorder mediated by the NPY Y2 receptor is selected from the group consisting of substance abuse and addiction related disorders.

16. A method as in claim 13, wherein the substance of abuse or addiction is alcohol.

17. A method of treating a disorder selected from the group consisting of anxiolytic disorders, depression; pain, injured mammalian nerve tissue; conditions responsive to treatment with a neurotrophic factor; neurological disorders; bone loss; cardiovascular diseases; sleep-wake state disorders, substance abuse and addiction related disorders; obesity; obesity-related disorders, disorders responsive to modulation of endocrine function, inovulation and infertility; comprising administering to a subject in need thereof, a therapeutically effective amount of a compound as in claim 1.

18. The use of a compound as in claim 1, for the preparation of a medicament for treating: (a) anxiolytic disorders, (b) depression; (c) pain, (d) injured mammalian nerve tissue; (d) conditions responsive to treatment with a neurotrophic factor; (e) neurological disorders; (f) bone loss; (g) cardiovascular diseases; (h) sleep-wake state disorders, (i) substance abuse and addiction related disorders; (j) obesity; (k) obesity-related disorders, (l) disorders responsive to modulation of endocrine function (more particularly, disorders responsive to modulation of the pituitary and/or hypothalamic gland); (m) inovulation; and (n) infertility; in a subject in need thereof.
